(19) United States
(12) Patent Application Publication
(10) Pub. No.: US 2026/0043790 A1
(43) Pub. Date: Feb. 12, 2026

(54) Skeletal Muscle-On-A-Chip In Microgravity As A Platform For Regeneration Modeling And Drug Screening

(71) Applicants: United States Government as represented by the Department of Veterans Affairs, Washington, DC (US); The Board Of Trustees Of The Leland Stanford Junior University, Stanford, CA (US)

(72) Inventors: Ngan F. Huang, Livermore, CA (US); Beu Oropeza, Palo Alto, CA (US)

(21) Appl. No.: 19/296,708

(22) Filed: Aug. 11, 2025

Related U.S. Application Data
(60) Provisional application No. 63/681,530, filed on Aug. 9, 2024.

(51) Int. Cl. G01N 33/50; C12M 1/00; C12M 1/12; C12M 3/00; C12M 3/06; C12N 5/077; G01N 33/68

(52) U.S. Cl. CPC G01N 33/5023; C12M 23/44; C12M 25/14; C12M 27/14; C12M 29/14; C12N 5/0658; G01N 33/5061; G01N 33/6887; C12N 2503/04; C12N 2513/00; C12N 2525/00; C12N 2533/54; G01N 2333/51; G01N 2333/521; G01N 2333/5446; G01N 2333/70578; G01N 2500/20

(57) ABSTRACT

Disclosed herein are systems and methods for testing effects of simulated microgravity on muscle structures. The system includes a clinostat and an adapter that is receivable into the clinostat. The adapter includes a housing that defines at least one receptacle. The system further includes an assembly having a substrate and a plurality of fibrils deposited on the substrate. The plurality of fibrils comprise collagen and extend along an axis. The assembly is receivable into a first receptacle of the at least one receptacle. The plurality of fibrils can have myoblasts thereon. Operation of the clinostat simulates microgravity. Test substances can screened using the muscle structures to determine effects on myogenesis.

### CROSS-REFERENCE TO RELATED APPLICATIONS

This application claims priority to and the benefit of the filing date of U.S. Provisional Patent Application No. 63/681,530, filed Aug. 9, 2024, the entirety of which is hereby incorporated by reference herein in its entirety.

### STATEMENT REGARDING FEDERALLY FUNDED RESEARCH

This invention was made with government support under R01 HL142718, R41 HL170875, R21 HL172096-01, R01 AG068667, and P01 AG036695 awarded by National Institutes of Health; 1I01BX004259, 5I01RX001222, 1I21RX004898, I01BX002324, I01RX001222, and IK6BX006309 awarded by the U.S. Department of Veterans Affairs; U.S. Pat. Nos. 1,829,534 and 2,227,614 awarded by the National Science Foundation; and 80JSC018M0005 awarded by the Center for the Advancement of Science in Space. The government has certain rights in the invention.

### FIELD

This disclosure relates to systems and methods for testing muscle growth and atrophy in simulated microgravity, as well as systems and methods for screening test substances based on contact between the test substances and engineered muscle tissue that has been exposed to microgravity (e.g., simulated microgravity).

### BACKGROUND

Individuals such as those in the aging population are susceptible to ailments, leading to hospitalization-induced unloading/disuse muscle atrophy. When older patients undergo hospitalization, there is an approximate 30-55% decline in ambulatory function and 65% decline in daily activities. Moreover, this decline in muscle conditioning during hospitalization leads to increased susceptibility to readmission after discharge. Importantly, there are no FDA-approved drugs to treat atrophy. Therefore, identifying interventions to treat disuse atrophy is a major public health challenge.

The development of interventions hinges on the ability to model or mimic disuse atrophy. Existing muscle disuse models include bed rest, dry immersion, or unilateral limb suspension induce muscle loss. However, such models rely primarily on in vivo subjects, which severely limit the ability to perform higher-throughput drug testing. In recent decades, the use of microgravity as a unique stimulus of muscle atrophy has gained traction. Assessments of structural and functional muscle changes during prolonged spaceflights or simulated microgravity have contributed to our knowledge of disuse atrophy. Under conditions of spaceflight, muscle cells undergo accelerated senescence and muscle mass is reduced dramatically over four weeks. Additionally, a larger body of work derived from in vitro cultured myoblasts in microgravity or simulated microgravity further support the finding that microgravity impairs myogenesis, which is a contributing factor to muscle atrophy. However, the opportunity to perform studies in real microgravity conditions is limited.

### SUMMARY

Disclosed herein, in various aspects, are systems and methods for testing effects of simulated microgravity on muscle structures.

In one aspect, the system includes a clinostat and an adapter that is receivable into the clinostat. The adapter comprises a housing that defines at least one receptacle. The system further comprises an assembly having a substrate and a plurality of fibrils deposited on the substrate. The plurality of fibrils comprise collagen and extend along an axis. The assembly is receivable into a first receptacle of the at least one receptacle.

Also disclosed herein is a method including the step of providing an adapter comprising a housing defining at least one receptacle, wherein the adapter is receivable into a clinostat. Each assembly of at least one assembly can be positioned into a respective receptacle of the at least one receptacle. For example, a first assembly can be positioned in a first receptacle of the at least one receptacle of the adapter. Each assembly of the at least one assembly comprises a substrate and a plurality of fibrils deposited on the substrate. The plurality of fibrils can comprise collagen and can extend along an axis. A first media can be flowed into the first receptacle.

Disclosed herein are methods for screening a test substance affecting myogenesis, the methods comprising: a) providing an engineered muscle tissue, wherein the engineered muscle tissue has been exposed to microgravity conditions (e.g., simulated microgravity conditions); b) contacting the engineered muscle tissue with the test substance; c) determining the expression of at least one mitochondrion gene or at least one biological process gene in the engineered muscle tissue after step b); and d) comparing the expression of the at least one mitochondrion gene or the at least one biological process gene in the engineered muscle tissue in step b) to the expression of the at least one mitochondrion gene or the at least one biological process gene in the engineered muscle tissue prior to step b), wherein a decrease in expression of the at least one mitochondrion gene indicates that the test substance increases myogenesis or wherein an increase in the expression of the at least one biological process gene indicates that the test substance increases myogenesis. Optionally, the engineered muscle tissue is contacted with the test substance during exposure of the engineered muscle tissue to microgravity conditions.

Disclosed herein are methods for identifying a test substance that increases muscle myogenesis, the methods comprising: a) contacting engineered muscle tissue with the test substance, wherein the engineered muscle tissue has been exposed to microgravity conditions (e.g., simulated microgravity conditions) prior to contacting the engineered muscle tissue with the test substance; b) determining the expression of at least one mitochondrion gene or at least one biological process gene in the engineered muscle tissue after the engineered muscle tissue is contacted with the test substance; and c) comparing the expression of the at least one mitochondrion gene or the at least one biological process gene in the engineered muscle tissue in step b) to the expression of the at least one mitochondrion gene or the at least one biological process gene in the engineered muscle tissue prior to step a), wherein a decrease in expression of the at least one mitochondrion gene indicates that the test substance increases myogenesis or wherein an increase in the expression of the at least one biological process gene indicates that the test substance increases myogenesis. Optionally, the engineered muscle tissue is contacted with the test substance during exposure of the engineered muscle tissue to microgravity conditions.

Disclosed herein are methods for screening a test substance affecting myogenesis, the methods comprising: a) providing an engineered muscle tissue, wherein the engineered muscle tissue has been exposed to microgravity conditions (e.g., simulated microgravity conditions); b) contacting the engineered muscle tissue with the test substance; c) immunofluorescently staining myosin heavy chain in the engineered muscle tissue, wherein the engineered muscle tissue comprises myotubes; and d) measuring myotube length or myotube width in the engineered muscle tissue via the expression of the myosin heavy chain in the myotubes, wherein an increase in myotube length or myotube width compared to the myotube length or myotube width prior to contacting the engineered muscle tissue with the test substance indicates that the test substance increases myogenesis. Optionally, the engineered muscle tissue is contacted with the test substance during exposure of the engineered muscle tissue to microgravity conditions.

Disclosed herein are methods for identifying a test substance that increases muscle myogenesis, the methods comprising: a) contacting engineered muscle tissue with the test substance, wherein the engineered muscle tissue has been exposed to microgravity conditions (e.g., simulated microgravity conditions) prior to contacting the engineered muscle tissue with the test substance; b) contacting the engineered muscle tissue with the test substance; c) immunofluorescently staining myosin heavy chain in the engineered muscle tissue, wherein the engineered muscle tissue comprises myotubes; and d) measuring myotube length or myotube width in the engineered muscle tissue via the expression of the myosin heavy chain in the myotubes, wherein an increase in myotube length or myotube width compared to the myotube length or myotube width prior to contacting the engineered muscle tissue with the test substance indicates that the test substance increases myogenesis. Optionally, the engineered muscle tissue is contacted with the test substance during exposure of the engineered muscle tissue to microgravity conditions.

Disclosed herein are methods of treating unloading/disuse atrophy in a subject in need thereof, the methods comprising administering to the subject a therapeutically effective amount of 15-hydroxyprostaglandin dehydrogenase (15-PGDH-I, SW033291), thereby treating unloading/disuse atrophy in the subject.

Disclosed herein are methods of reducing or ameliorating one or more symptoms of unloading/disuse atrophy in a subject in need thereof, the methods comprising administering to the subject a therapeutically effective amount of 15-hydroxyprostaglandin dehydrogenase (15-PGDH-I, SW033291), thereby reducing or ameliorating one or more symptoms of unloading/disuse atrophy in the subject.

These and other objects and features of the present invention will become more fully apparent from the following description and appended claims, or may be learned by the practice of the invention as set forth hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-IE show the generation and characterization of a biomimetic skeletal muscle-on-a-chip platform. FIG. 1A shows a schematic depicting the fabrication of aligned collagen nanofibrils using shear-mediated extrusion of monomeric collagen into a pH 7 buffer. FIG. 1B shows a gross image and schematic of anisotropic collagen strips deposited onto hydrophobic glass chips (scale bar=5 mm). FIG. 1C shows a scanning electron microscopy analysis with highly organized anisotropic collagen fibrils (scale bar=1 μm). FIG. 1D shows a seeding of primary human skeletal muscle cells with cell alignment along the direction of the collagen nanofibrils, as denoted by the arrow (scale bar=100 μm). FIG. 1E shows that, in induction media, the cells can fuse to form parallel-aligned myotubes, as indicated by the green myosin heavy chain staining (scale bar=100 μm).

FIG. 2 shows the preparation timeline for muscle-on-a-chip devices for microgravity studies and an experimental overview. As shown at (A), the muscle-on-a-chip preparation consists of seeding human muscle cells onto immobilized aligned nanofibrillar collagen scaffolds followed by cell seeding. After one day, the cell-seeded scaffolds are placed into bioreactors and filled with growth media. The bioreactor is then secured with screws for transportation to the International Space Station National Laboratory (“ISSNL”). As shown at (B), the experiment comprises incubating the bioreactor during transit to the ISSNL in plate habitats that provide temperature maintenance or in normal gravity conditions on Earth. Upon docking at the ISSNL, microgravity experiments began (day 0) with media change into an induction media that supports myotube formation. In some experiments, drugs were added to the media on day 0 and again on day 3. On day 7, the conditioned media was extracted, and the samples were stabilized in RNA or fixed in paraformaldehyde for analysis by RNA Sequencing, proteomics analysis, or myotube formation.

FIGS. 3A-3F shows customized bioreactors for a muscle-on-a-chip platform in microgravity. FIG. 3A shows a BioCell bioreactor that houses an engineered muscle-on-a-chip, which can be secured to the bioreactor using screws. FIG. 3B shows the bioreactor filled with media. FIG. 3C shows the bioreactors housed within plate habitats that provide temperature maintenance during launch and in microgravity. FIG. 3D shows how the media can be changed in microgravity using syringes that exchange media through ports in the bioreactor. FIG. 3E shows how media exchange can be performed in a life sciences glovebox. FIG. 3F shows how astronauts look at engineered muscle-on-a-chip bioconstructs under a microscope.

FIGS. 4A-4B show the quantification of myotube formation in muscle-on-a-chip bioconstructs in microgravity. FIG. 4A shows myotube length, width, and index quantification in muscle-on-a-chip bioconstructs derived from donors 1 and 2. Data is shown as mean±standard deviation. FIG. 4B is a table showing, for each of two donor muscle-on-a-chip bioconstructs, that five confocal images were analyzed and averaged out of five images per group.

FIGS. 5A-5G show transcriptional differences between microgravity and gravity control biomimetic skeletal muscle. FIG. 5A shows principal component analysis of the transcriptome of biomimetic skeletal muscle samples after 7 days in microgravity or control conditions (gravity). FIG. 5B shows a heatmap of all differentially expressed genes after 7 days in microgravity, compared to gravity (FDR 10%). FIG. 5C shows the gene ontology (GO) enrichment terms of differentially upregulated genes in microgravity (P<0.05). FIG. 5D shows a heatmap for the expression of genes found in the mitochondrion GO enrichment term. FIG. 5E shows the pathway enrichment terms of differentially upregulated genes in microgravity (P<0.05). FIG. 5F shows the pathway enrichment terms of differentially downregulated genes in microgravity (P<0.05). FIG. 5G shows a heatmap for the expression of the top 20 differentially downregulated genes in microgravity (n=2 donors per group).

FIG. 6 shows protein and RNA differences in biomimetic skeletal muscle bioconstructs in microgravity, compared to gravity conditions. The symbol and line plots show differentially expressed proteins in microgravity versus gravity biomimetic skeletal muscle conditioned media and their corresponding RNA levels from bulk RNA sequencing. Microgravity (flight) or gravity (ground) conditions are denoted, in which lines are associated with the same donor samples. The proteins and corresponding RNA samples that show similar expression level changes are highlighted in a dotted box. The data is derived from two donor-derived muscle-on-a-chip bioconstructs per condition.

FIG. 7 shows a protein-protein interaction (“PPI”) network for space flight biomimetic skeletal muscle. The inset highlights GDF-15 and an immediate protein interacting with GDF-15 magnified on the right.

FIGS. 8A-8F show transcriptional similarities between clinical sarcopenia muscle and engineered skeletal muscle in microgravity. FIG. 8A shows a principal component analysis of the transcriptome in healthy versus sarcopenia clinical samples from both sexes. FIG. 8B shows a volcano plot indicating differentially expressed genes in sarcopenia, compared to normal conditions (P<0.05, log 2 FC>1.0). Genes involved in DNA damage response, extracellular matrix organization, VEGFA-VEGFR2 signaling pathway, and Wnt signaling are labeled. Genes showing statistically significant expression (FDR 25%) are indicated in red. FIG. 8C shows a heatmap with the genes involved in Wnt signaling, rhabdomyosarcoma, DNA damage response, extracellular matrix organization, and VEGFA-VEGFR2 signaling. FIGS. 8D and 8E show enrichment plots for custom gene set enrichment analysis (“GSEA”) (The red curve indicates positive enrichment, whereas the blue curve indicates negative enrichment scores. Black bars indicate the positions of the gene set on the rank ordered list in GSEA, and the signal to noise metric are shown at the bottom.). FIG. 8D shows positive enrichment of the Old_MuSC_UP gene set in microgravity, compared to gravity samples. FIG. 8E shows negative enrichment of Sarcopenia_UP and Sarcopenia_DOWN gene sets in microgravity, compared to gravity samples. FIG. 8F is a table showing demographics of total RNA samples obtained from clinical sarcopenia or healthy muscle tissue donors.

FIGS. 9A-9I show how IGF-1 and 15-PDGFH inhibitor (15-PDGH-i) treatment can partially prevent microgravity effect. FIG. 9A shows a principal component analysis of transcriptome in 7 days in microgravity or gravity control conditions in the presence of drug treatment (IGF-1 or 15-PDGH-i) in biomimetic skeletal muscle samples. FIG. 9B shows a 4-way Venn-diagram illustrating the number of differentially expressed genes that were upregulated in microgravity, compared to gravity conditions, or whose differential expression could be either prevented by IGF-1 or 15-PDGH-i treatment (gray) or were not prevented by drug treatment (IGF-1 in blue and 15-PDGH-i in orange). FIG. 9C shows a 4-way Venn-diagram illustrating the number of differentially expressed genes downregulated in microgravity, compared to gravity conditions, or whose differential expression could be either prevented by IGF-1 or 15-PDGH-i treatment (gray) or were not prevented by drug treatment (IGF-1 in blue and 15-PDGH-i in orange). FIG. 9D shows a heatmap of genes implicated in lipid biosynthesis, metabolic pathways, and adipogenesis. FIG. 9E shows a heatmap for genes implicated in cell adhesion, Notch signaling, and Wnt signaling (n=2 donors per group). FIG. 9F shows a heatmap for genes implicated in lipid biosynthesis, metabolic pathways, and adipogenesis. FIGS. 9G-9I show proteins and RNAs whose differential expression could be prevented by IGF-1 or 15-PDGH-i treatment. FIGS. 9G-9H show symbol and line plots for proteins differentially expressed with microgravity and their levels in biomimetic skeletal muscle conditioned media with drug treatment (IGF-1 or 15-PDGH-i). FIG. 9I shows symbol and line plots for EGR1 and TRIB3 RNA levels in microgravity, gravity, and drug treated samples. Symbols are colored by microgravity, gravity, and drug treated conditions, in which lines join the same donor samples. The data is derived from two donor-derived muscle-on-a-chip bioconstructs per condition.

FIG. 10 shows a bioreactor in simulated microgravity setup. The bioreactor (BioCell) can allow for tissue culture conditions to be maintained under a microgravity environment, as shown at (A). The adapter and subsequent covers can allow this system to be used within the clinostat while maintaining a balanced system for optimal microgravity simulation, as shown at (B).

FIG. 11 shows the varying effects of compounds on myotube formation. Representative images show the effects of the compounds on fusion and morphology of the myotubes in simulated microgravity. Nuclei (blue) and MHC (green) can be seen for all images (scale bars represent 100 μm).

FIGS. 12A-12C show the quantification of myotube characteristics of HMCLs treated with the 8 different compounds at varying concentrations that indicate a 10% increase in myotube formation. FIG. 12A shows the mean myotube lengths. FIG. 12B shows the mean myotube widths. FIG. 12C shows the fusion index.

FIG. 13 shows a schematic overview of the specific aims of Example 3.

FIGS. 14A-14C show in vitro characterization of myotube formation, organization, and function in engineered skeletal muscle construct. FIG. 14A shows scanning electron microscopy images of randomly oriented or aligned nanofibrillar scaffolds. FIG. 14B shows confocal microscopy images of myosin heavy chain (“MHC”) for myotubes within engineered skeletal muscle (double arrow denotes orientation of nanofibril alignment). FIG. 14C is a histogram of cell contractility in the randomly oriented scaffold or aligned scaffold upon electrical stimulation.

FIG. 15 shows how microgravity can inhibit myotube formation in an engineered muscle-on-a-chip. Samples were exposed to gravity or microgravity conditions for 7 days and immunofluorescently stained for myosin heavy chain.

FIG. 16 shows the fabrication of nanofibrillar scaffolds with an extrusion of monomeric collagen at a high syringe velocity to form aligned nanofibrillar collagen.

FIG. 17 shows the daily injection of SW033291 in aged mice promoted the formation of larger muscle fibers, including an image showing the immunostaining of laminin depicting fiber size, a histogram of muscle fiber size distribution, and a mean cross sectional area of myofibers.

FIGS. 18A-18B show schematic and time courses for experiments in Example 3. FIG. 18A depicts drug testing in real microgravity. FIG. 18B depicts MPC seeding onto scaffolds in simulated microgravity or normal gravity conditions.

FIG. 19 shows how the treatment of SW033291 for 7 days can partially prevent atrophy-like qualities in engineered muscle-on-a-chip constructs in microgravity. The heatmap at (A) shows genes implicated in lipid biosynthesis, metabolic pathways, and adipogenesis that are upregulated in microgravity, compared to gravity, but reversed by treatment using SW033291. The heatmap at (B) shows genes downregulated in microgravity, but mitigated by drug treatment.

FIG. 20 is a table showing an exemplary timeline of Example 3.

FIG. 21 is a graphical representation showing quantification of myotube characteristics in a well plate environment—Lengths. The graphical representation of the myotube characteristics analysis showing the distribution of myotube lengths of HMCLs treated with the different compounds at varying concentrations. This system can be adapted for high-throughput data acquisition when a 96 well-plate and the corresponding adapter is used.

FIG. 22 is a graphical representation showing quantification of myotube characteristics in a well plate environment—Widths. The graphical representation of the myotube characteristics analysis showing the distribution of myotube widths of HMCLs treated with varying compounds.

FIG. 23 shows images illustrating effects of simulated microgravity and normal gravity on myotube formation in a nanopatterned scaffold environment. Representative images showing the effects of the compounds on fusion and morphology of the myotubes. The disclosed adaptors were necessary, allowing for the collagen scaffolds to be used. Nuclei (blue) and MHC (green) can be seen for all images. Scale bars represent 100 μm.

FIGS. 24A and 24B show quantification of myotube characteristics in a nanopatterned scaffold environment. The graphical representation of the myotube characteristics analysis showing the mean myotube lengths (FIG. 24A) and widths (FIG. 24B) of HMCLs treated with the different compounds atop a nanopatterned collagen scaffold in both simulated microgravity and normal gravity. The disclosed adaptors were necessary, allowing for the collagen scaffolds to be used.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of” For example, “at least one of A, B and C” explicitly includes only A, only B, only C, and combinations of each.

As used in the specification and in the claims, the term “comprising” can include the aspects “consisting of” and “consisting essentially of” “Comprising” can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and, unless the context dictates otherwise, can, in alternative aspects, also include any combination of members of that list.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. The subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder (e.g., disuse atrophy, sarcopenia, neurogenic atrophy, disease-related atrophy). The term “patient” includes human and veterinary subjects. In some aspects of the disclosed methods, the “patient” has been diagnosed with a need for treatment for increasing myogenesis or any one of disuse atrophy, sarcopenia, neurogenic atrophy, or disease-related atrophy, such as, for example, prior to the administering step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100%, or more, or any amount of promotion in between compared to native or control levels. In an aspect, the increase or promotion is 10-20, 20-30, 30-40, 40-50, 50-60, 60-70, 70-80, 80-90, or 90-100% as compared to native or control levels. In an aspect, the increase or promotion is 0-25, 25-50, 50-75, or 75-100%, or more, such as 200, 300, 500, or 1000% more as compared to native or control levels. In an aspect, the increase or promotion can be greater than 100 percent as compared to native or control levels, such as 100, 150, 200, 250, 300, 350, 400, 450, 500% or more as compared to the native or control levels or animal disease model.

As used herein, the term “determining” can refer to measuring or ascertaining a quantity or an amount or a change in activity. For example, determining the amount or expression of a disclosed gene or protein in a sample as used herein can refer to the steps that the skilled person would take to measure or ascertain some quantifiable value of the gene or protein in the sample. The art is familiar with the ways to measure an amount of the disclosed proteins, genes and nucleotides in a sample.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, affection.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment can also be administered to a subject to ameliorate one more signs of symptoms of a disease, disorder, and/or condition. For example, the disease, disorder, and/or condition can be relating to myogenesis.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, use of “substantially” (e.g., “substantially parallel”) or “generally” (e.g., “generally planar”) should be understood to include embodiments in which angles are within ten degrees, or within five degrees, or within one degree.

The phrase “at least” preceding a series of elements is to be understood to refer to every element in the series. For example, “at least one” includes one, two, three, four or more.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

Disclosed herein, in various aspects, are devices, systems, and methods for testing effects of microgravity on muscle atrophy in real or simulated microgravity. Real microgravity can include environmental factors experienced on body in orbit, such as that on the International Space Station (e.g., on the order of 10-3 g or less). Simulated microgravity can include generated environmental factors that cause effects that are representative of effects caused under real microgravity conditions. For example, microgravity can be simulated with a clinostat that effects rotation of a sample about one or more axes. When provided on a clinostat, the sample can act in a manner representative of the sample being provided in real microgravity. More particularly, in some exemplary aspects, the disclosed systems and methods can be used for drug screening performed on engineered muscle tissue under simulated microgravity.

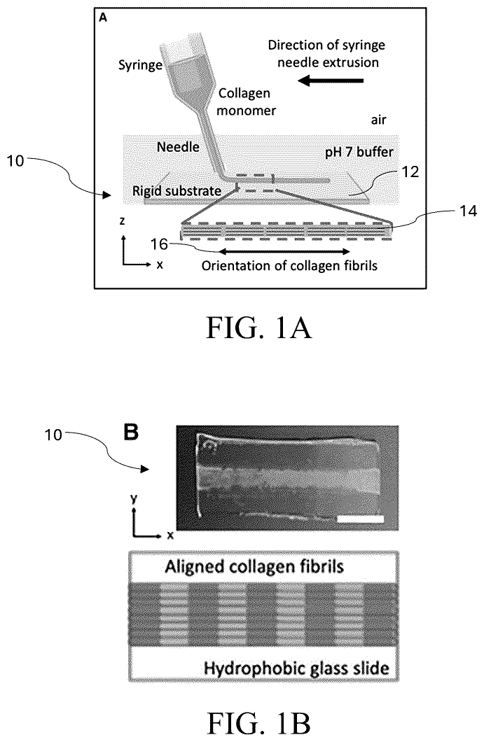

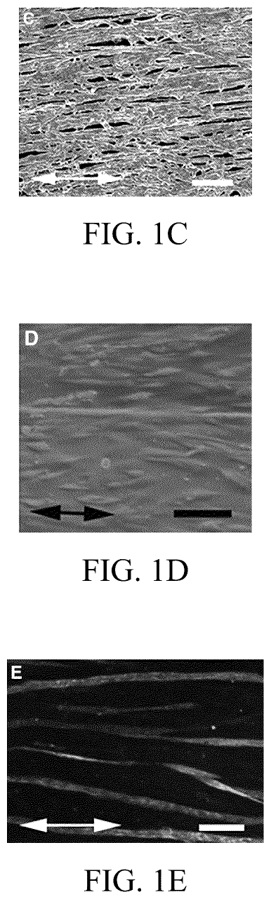

Referring to FIGS. 1A and 1B, an assembly 10 for preparing a sample can comprise a substrate 12 and a plurality of fibrils 14 deposited on the substrate. The plurality of fibrils 14 can comprise collagen. The plurality of fibrils extend along an axis 16. The fibrils can serve as a scaffolding for growth of myoblasts that simulate muscle fibers. For example, the fibrils having cells seeded thereon can be representative of a bundle of muscle fibers. Thus, in exemplary aspects, it is contemplated that the prepared sample can be an engineered muscle tissue as further disclosed herein.

In some aspects, the substrate can comprise hydrophobic glass. In some aspects, the collagen can be acidic monomeric collagen type I. In some optional aspects, each fibril of the plurality of fibrils can have a diameter of less than 100 nm. In some aspects, the collagen can be extruded to form the plurality of fibrils. For example, the acidic monomeric collagen type I can be sheared from a blunt needle to form the fibrils. The substrate can be bathed in a pH neutral buffer.

A method of preparing a sample can comprise applying a plurality of cells to the plurality of fibrils of the assembly. The plurality of cells can be myoblasts (e.g., myoblasts of skeletal muscle cells, such as, for example, human muscle cells).

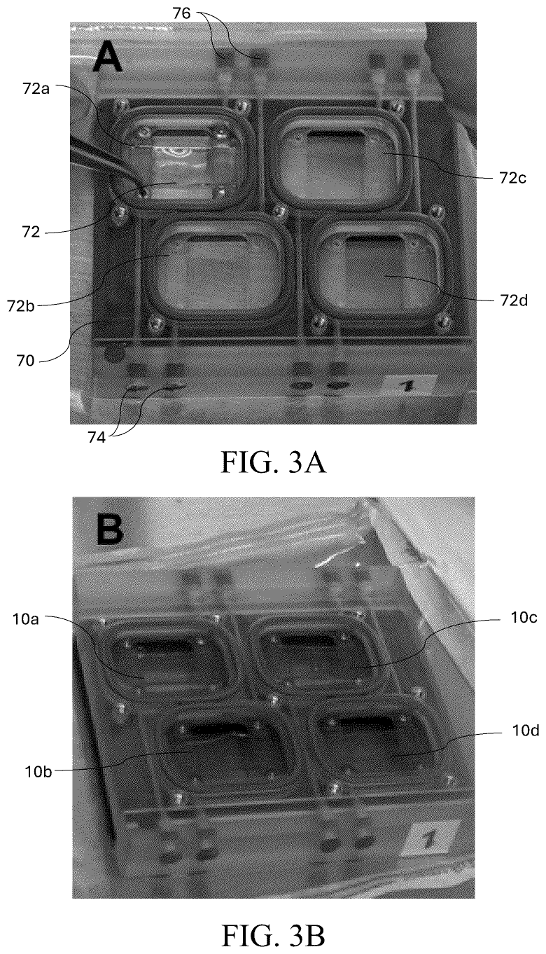

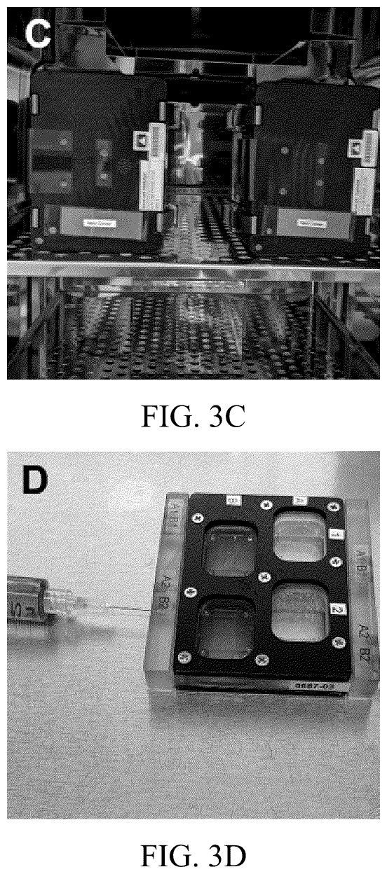

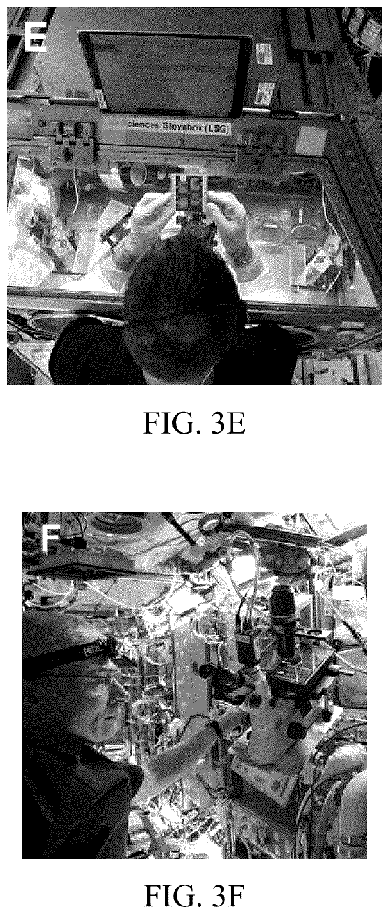

Referring to FIGS. 3D, 10, and 13, the sample can be tested under simulated microgravity using a clinostat 50. In some aspects, the clinostat can be configured to simulate microgravity on the order of 10⁻³ g or less. For example, the clinostat 50 can receive an adapter 60. The adapter 60 can comprise a housing 70 that defines at least one receptacle 72 (optionally, a plurality of receptacles). A first assembly 10a can be received within a first receptacle 72a of the at least one receptacle of the adapter. A first media can be flowed into the first receptacle. The adapter 60 can be positioned within the clinostat 50. The clinostat 50 can rotate the first assembly about at least one axis (optionally, a first axis and a second axis that is perpendicular to the first axis) to simulate microgravity.

In some aspects, the housing 70 can receive a plurality of assemblies 10. For example, a second assembly 10b can be positioned in a second receptacle 72b of the at least one receptacle of the adapter, and a second media can be flowed into the second receptacle. In further aspects, third and fourth assemblies 10c,d can be positioned in third and fourth receptacles 72c,d, respectively. Third and fourth media can be flowed into the third and fourth receptacles, respectively. In some aspects, each or some of the first, second, third, and fourth media can be different. In this way, the different media can be compared to determine their respective effects on muscle tissue in microgravity. The media can have nutrients for growth of muscle cells. The media can further have drugs, chemicals, or other treatment compounds that can have an effect on muscle cell growth or inhibition of muscle cell atrophy. Specific, non-limiting examples of suitable media are provided in the Examples section of this specification.

The first assembly 10a can have a plurality of myoblasts seeded on the plurality of fibrils. For example, the plurality of myoblasts seeded on the plurality of fibrils cooperate to form a plurality of different samples. The plurality of myoblasts of the different samples can differ. For example, the myoblasts can have different DNA.

A system 80 can comprise the clinostat 50, the adapter 60, and the assembly 10, wherein the assembly is received into the first receptacle 72a of the at least one receptacle of the housing 70. In aspects in which the housing 70 defines a plurality of receptacles 72, the housing can further define a plurality of fluid inlet conduits 74. A respective fluid conduit of the plurality of fluid conduits can be in communication with a corresponding receptacle of the plurality of receptacles. Each fluid inlet conduit 74 can be configured to receive fluid therethrough to permit the respective receptacle to be filled with media. The housing can further define a plurality of outlet conduits 76. A respective outlet conduit 76 can be in communication with a corresponding receptacle of the plurality of receptacles 72. The outlet conduits 76 can permit the fluid (e.g., air or media) to be displaced from the plurality of receptacles 72 to permit flow from the inlet conduits 74.

### Methods of Screening

Disclosed herein are methods, systems, and devices useful for screening drugs or candidate drugs. Also disclosed herein are methods useful for treating patient populations.

Disclosed herein are methods for screening a test substance affecting myogenesis. Also disclosed herein are methods for identifying a test substance that increases muscle myogenesis.

Disclosed herein are method for screening a test substance affecting myogenesis. In some aspects, the methods can comprise: a) providing an engineered muscle tissue, wherein the engineered muscle tissue has been exposed to microgravity conditions (e.g., simulated microgravity conditions); b) contacting the engineered muscle tissue with the test substance; c) determining the expression of at least one mitochondrion gene or at least one biological process gene in the engineered muscle tissue after step b); and d) comparing the expression of the at least one mitochondrion gene or the at least one biological process gene in the engineered muscle tissue in step b) to the expression of the at least one mitochondrion gene or the at least one biological process gene in the engineered muscle tissue prior to step b). In some aspects, a decrease in expression of the at least one mitochondrion gene indicates that the test substance increases myogenesis. In some aspects, an increase in the expression of the at least one biological process gene indicates that the test substance increases myogenesis. Optionally, the engineered muscle tissue is contacted with the test substance during exposure of the engineered muscle tissue to microgravity conditions.

Disclosed herein are methods for screening a test substance affecting myogenesis. In some aspects, the methods can comprise: a) providing an engineered muscle tissue, wherein the engineered muscle tissue has been exposed to microgravity conditions (e.g., simulated microgravity conditions); b) contacting the engineered muscle tissue with the test substance; c) immunofluorescently staining myosin heavy chain in the engineered muscle tissue, wherein the engineered muscle tissue comprises myotubes; and d) measuring myotube length or myotube width in the engineered muscle tissue via the expression of the myosin heavy chain in the myotubes. In some aspects, an increase in myotube length or myotube width compared to the myotube length or myotube width prior to contacting the engineered muscle tissue with the test substance indicates that the test substance increases myogenesis. Optionally, the engineered muscle tissue is contacted with the test substance during exposure of the engineered muscle tissue to microgravity conditions.

Disclosed herein are methods for identifying a test substance that increases muscle myogenesis. In some aspects, the methods can comprise: a) contacting engineered muscle tissue with the test substance, wherein the engineered muscle tissue has been exposed to microgravity conditions (e.g., simulated microgravity conditions) prior to contacting the engineered muscle tissue with the test substance; b) determining the expression of at least one mitochondrion gene or at least one biological process gene in the engineered muscle tissue after the engineered muscle tissue is contacted with the test substance; and c) comparing the expression of the at least one mitochondrion gene or the at least one biological process gene in the engineered muscle tissue in step b) to the expression of the at least one mitochondrion gene or the at least one biological process gene in the engineered muscle tissue prior to step a). In some aspects, a decrease in expression of the at least one mitochondrion gene indicates that the test substance increases myogenesis. In some aspects, an increase in the expression of the at least one biological process gene indicates that the test substance increases myogenesis. Optionally, the engineered muscle tissue is contacted with the test substance during exposure of the engineered muscle tissue to microgravity conditions.

Disclosed herein are methods for identifying a test substance that increases muscle myogenesis. In some aspects, the method can comprise: a) contacting engineered muscle tissue with the test substance, wherein the engineered muscle tissue has been exposed to microgravity conditions (e.g., simulated microgravity conditions) prior to contacting the engineered muscle tissue with the test substance; b) contacting the engineered muscle tissue with the test substance; c) immunofluorescently staining myosin heavy chain in the engineered muscle tissue, wherein the engineered muscle tissue comprises myotubes; and d) measuring myotube length or myotube width in the engineered muscle tissue via the expression of the myosin heavy chain in the myotubes. In some aspects, an increase in myotube length or myotube width compared to the myotube length or myotube width prior to contacting the engineered muscle tissue with the test substance indicates that the test substance increases myogenesis. Optionally, the engineered muscle tissue is contacted with the test substance during exposure of the engineered muscle tissue to microgravity conditions.

In any of the methods disclosed herein, the engineered muscle tissue can be engineered skeletal muscle tissue. In some aspects, the engineered muscle tissue can comprise a plurality of myoblasts.

In some aspects, the microgravity conditions (e.g., simulated microgravity conditions) can comprise exposing the engineered muscle tissue to 10⁻³ g or less. In some aspects, the microgravity conditions (e.g., simulated microgravity conditions) can comprise exposing the engineered muscle tissue to 10⁻³ g to 10⁻⁶ g. In some aspects, the microgravity conditions (e.g., simulated microgravity conditions) can comprise exposing the engineered muscle tissue to 10⁻³ g for 7 to 14 days. In some aspects, the microgravity conditions (e.g., simulated microgravity conditions) can comprise exposing the engineered muscle tissue to 10⁻³ g or less for 1, 2, 3, 4, 5, 6, 7, 8, 9, 10, 11, 12, 13, or 14 days. In some aspects, the microgravity conditions (e.g., simulated microgravity conditions) can comprise exposing the engineered muscle tissue to 10⁻² g or less for 1, 2, 3, 4, 5, 6, 7, 8, 9, 10, 11, 12, 13, or 14 days.

In any of the methods disclosed herein, the at least one mitochondrion gene can be selected from the group of mitochondrially encoded cytochrome c oxidase III (MT-CO3), fumarylacetoacetate hydrolase domain-containing protein 1 (FAHD1), lon protease homolog (LONP1), phosphoenolpyruvate carboxykinase 2 (PCK2), glutaredoxin-1 (GLRX), RAB32, Bcl-2 related ovarian killer (BOK), cytochrome C oxidase assembly factor 4 homolog (COA4), dehydrogenase/reductase 4 (DHRS4), fatty acid desaturase 1 (FADS1), EF-hand domain family member D1 (EFHD1), NX4, MTHFD2, and serum/glucocorticoid regulated kinase 1 (SGK1).

In any of the methods disclosed herein, the at least one biological process gene can be selected from the group consisting of protocadherin gamma C3 (PDCHGC3), prostaglandin-endoperoxide synthase 1 (PTGS1), transforming growth factor beta induced (TGFB1), NYN domain and retroviral integrase containing (NYNRIN), neuroligin 2 (NLGN2),hemicentin-1 (HMCN1), dystonin (DST), zinc finger and BTB domain-containing 20 (ZBTB20), laminin alpha 2 (LAMA2), collagen type XII alpha 1 chain (COL12A1), proline rich coiled-coil 2C (PRRC2C), nuclear factor of activated T-cells 5 (NFAT5), golgin subfamily B member 1 (GOLGB1), SCUBE3, zinc finger protein 469 (ZNF469), heparan sulfate proteoglycan 2 (HSPG2), PH and SEC7 domain-containing protein 3 (PSD3), cluster of differentiation 109 (CD109), neuroblast differentiation-associated protein AHNAK (AHNAK), and neurogenic locus notch homolog protein 2 (NOTCH2).

In any of the methods disclosed herein, the methods can further comprise performing a proteomic analysis of the engineered muscle tissue. In some aspects, the methods can further comprise performing a proteomic analysis of the engineered muscle tissue before microgravity exposure, after microgravity exposure, during any time point during microgravity exposure, after microgravity exposure but before contacting the engineered muscle tissue with the test substance, or after microgravity exposure and after contacting the engineered muscle tissue with the test substance. In some aspects, the proteomic analysis can comprise determining the amount of at least one protein. In some aspects, the proteomic analysis can comprise determining the amount of at least one protein selected from the group consisting of Eotaxin-3 (CCL26), C-X-C motif chemokine ligand 16 (CXCL16), growth differentiation factor 15 (GDF-15), tumor necrosis factor superfamily member 14 (LIGHT/TNSF14), and pupoid fetus (PF). In some aspects, when the amount of Eotaxin-3 (CCL26), C-X-C motif chemokine ligand 16 (CXCL16), growth differentiation factor 15 (GDF-15), tumor necrosis factor superfamily member 14 (LIGHT/TNSF14), or pupoid fetus (PF) is decreased compared to the amount of the same protein present prior to contacting the engineered muscle tissue with the test substance it indicates that the test substance increases myogenesis. In some aspects, when the amount of the at least one protein is decreased compared to the amount of the same protein present prior to contacting the engineered muscle tissue with the test substance it indicates that the test substance increases myogenesis. In some aspects, the proteomic analysis can comprise determining the amount of at least one protein selected from the group consisting of bone morphogenetic protein 4 (BMP-4), Resistin, C-X-C motif chemokine ligand 12 (CXCL12/SDF-1b), interleukin-16 (IL-16), and CD40. In some aspects, when the amount of bone morphogenetic protein 4 (BMP-4), Resistin, C-X-C motif chemokine ligand 12 (CXCL12/SDF-1b), interleukin-16 (IL-16), or CD40 is increased compared to the amount of the same protein present prior to contacting the engineered muscle tissue with the test substance it indicates that the test substance increases myogenesis.

In any of the methods disclosed herein, the methods can further comprise immunofluorescently staining myosin heavy chain in the engineered muscle tissue. In some aspects, immunofluorescently staining myosin heavy chain in the engineered muscle tissue before microgravity exposure, after microgravity exposure, during any time point during microgravity exposure, after microgravity exposure but before contacting the engineered muscle tissue with the test substance, or after microgravity exposure and after contacting the engineered muscle tissue with the test substance. wherein the engineered muscle tissue comprises myotubes. In some aspects, the engineered muscle tissue can comprise myotubes.

In any of the methods disclosed herein, the method can further comprise measuring myotube length or myotube width in the engineered muscle tissue via the expression of the myosin heavy chain in the myotubes. In some aspects, can be compared to the myotube length or myotube width prior to contacting the engineered muscle tissue with the test substance. In some aspects, an increase in myotube length or myotube width compared to the myotube length or myotube width prior to contacting the engineered muscle tissue with the test substance indicates that the test substance increases myogenesis.

In any of the methods disclosed herein, the method can further comprise counting the number of nuclei per myotube in the engineered muscle tissue before contacting the engineered muscle tissue with the test substance and after contacting the engineered muscle tissue with the test substance. In some aspects, the methods can comprise comparing the number of nuclei per myotube in the engineered muscle tissue. In some aspects, the methods can comprise comparing the number of nuclei per myotube in the engineered muscle tissue, wherein an increase in the number of nuclei per myotube in the engineered muscle tissue after contacting the engineered muscle tissue with the test substance indicates that the test substance increases myogenesis.

In any of the methods disclosed herein, the method can further comprise calculating a fusion index. In some aspects, when the fusion index is increased after contacting the engineered muscle tissue indicates that the test substance increases myogenesis. In some aspects, the fusion index can be typically calculated by: determining the total number of nuclei: this includes all nuclei in the sample, whether they are in unfused myoblasts or fused myotubes; determining the number of nuclei within myotubes: this involves identifying and counting the nuclei within cells that have fused into larger, multinucleated structures; and calculating the ratio: the fusion index is then calculated as (number of nuclei in myotubes)/(total number of nuclei)*100. A fusion index (FI) is a metric used in cell biology, particularly in studies of myogenesis (muscle cell development), to quantify the extent of cell fusion. It represents the proportion of nuclei within fused cells (myotubes) compared to the total number of nuclei in a sample. In some aspects, it indicates how effectively mononucleated muscle cells (myoblasts) have fused together to form multinucleated muscle fibers (myotubes). In some aspects, drugs or test substances can be screened and analyzed using one, two, or three different metrics: mean myotube length, mean myotube width, and a fusion index which describes the percentage of nuclei that can be found inside of a myotube in relation to the overall number of nuclei in an image. In some aspects, any drug or test substance found showing >10% increase that those found in the control group can be selected to be subjected to additional studies in order to validate the results.

In any of the methods disclosed herein, the methods can further comprise performing RNA sequencing analysis after contacting the engineered muscle tissue with the test substance.

In any of the methods disclosed herein, the methods can further comprise comparing the expression of at least one gene in the engineered muscle tissue with the expression of the same gene in the engineered muscle tissue after contacting the engineered muscle tissue with a reference compound. In some aspects, the gene can be a mitochondrion gene or the at least one biological process gene. In some aspects, the methods can further comprise comparing the expression of the at least one mitochondrion gene or the at least one biological process gene in the engineered muscle tissue with the expression of the same at least one mitochondrion gene or the at least one biological process gene in the engineered muscle tissue after contacting the engineered muscle tissue with a reference compound. In some aspects, the reference compound can be insulin-like growth factor-1 or 15-hydroxyprostaglandin dehydrogenase (SW033291). In some aspects, the gene or the at least one mitochondrion gene can be selected from the group of mitochondrially encoded cytochrome c oxidase III (MT-CO3), fumarylacetoacetate hydrolase domain-containing protein 1 (FAHD1), lon protease homolog (LONP1), phosphoenolpyruvate carboxykinase 2 (PCK2), glutaredoxin-1 (GLRX), RAB32, Bcl-2 related ovarian killer (BOK), cytochrome C oxidase assembly factor 4 homolog (COA4), dehydrogenase/reductase 4 (DHRS4), fatty acid desaturase 1 (FADS1), EF-hand domain family member D1 (EFHD1), NX4, MTHFD2, and serum/glucocorticoid regulated kinase 1 (SGK1). In some aspects, the gene or the at least one biological process gene can be selected from the group consisting of protocadherin gamma C3 (PDCHGC3), prostaglandin-endoperoxide synthase 1 (PTGS1), transforming growth factor beta induced (TGFB1), NYN domain and retroviral integrase containing (NYNRIN), neuroligin 2 (NLGN2),hemicentin-1 (HMCN1), dystonin (DST), zinc finger and BTB domain-containing 20 (ZBTB20), laminin alpha 2 (LAMA2), collagen type XII alpha 1 chain (COL12A1), proline rich coiled-coil 2C (PRRC2C), nuclear factor of activated T-cells 5 (NFAT5), golgin subfamily B member 1 (GOLGB1), SCUBE3, zinc finger protein 469 (ZNF469), heparan sulfate proteoglycan 2 (HSPG2), PH and SEC7 domain-containing protein 3 (PSD3), cluster of differentiation 109 (CD109), neuroblast differentiation-associated protein AHNAK (AHNAK), and neurogenic locus notch homolog protein 2 (NOTCH2).

In any of the methods disclosed herein, the methods can further comprise performing a muscle contractility assessment of the engineered muscle tissue. In some aspects, the muscle contractility assessment of the engineered muscle tissue can be performed before microgravity exposure, after microgravity exposure, during any time point during microgravity exposure, after microgravity exposure but before contacting the engineered muscle tissue with the test substance, or after microgravity exposure and after contacting the engineered muscle tissue with the test substance. In some aspects, the methods can comprise comparing the muscle contractility of the engineered muscle tissue before contacting the engineered muscle tissue with the test substance and after contacting the engineered muscle tissue with the test substance. In some aspects, the methods can comprise comparing the muscle contractility of the engineered muscle tissue before contacting the engineered muscle tissue with the test substance and after contacting the engineered muscle tissue with the test substance, wherein an increase in muscle contractility of the engineered muscle tissue after contacting the engineered muscle tissue with the test substance indicates that the test substance increases myogenesis.

In any of the methods disclosed herein, the test substance can be selected from the group consisting of an agent for treating or preventing disuse atrophy, an agent for treating or preventing sarcopenia, an agent for treating or preventing neurogenic atrophy, or an agent for treating or preventing disease-related atrophy. In some aspects, the test substance can be selected from the group consisting of an agent for treating or preventing disease associated with a decrease of slow-twitch muscle fibers, an agent for treating or preventing disease associated with an increase of in slow-twitch muscle fibers, an agent for treating or preventing disease associated with a decrease of fast-twitch muscle fibers, or an agent for treating or preventing disease associated with an increase of fast-twitch muscle fibers. In some aspects, the disease-related atrophy can be cancer, chronic obstructive pulmonary disorder, diabetes, chronic kidney disease, heart failure, or a neurodegenerative disorder. In some aspects, the test substance can be an FDA-approved repurposed drug. In some aspects, the test substance is not insulin-like growth factor-1. In some aspects, the test substance is not 15-hydroxyprostaglandin dehydrogenase (15-PGDH-I, SW033291).

### Methods of Treating

Disclosed herein are methods of treating a subject or patient in need thereof. In some aspects, the subject or patient can be a human. In some aspects, the subject can be identified in need of treatment before the administering step.

Disclosed herein are methods of treating unloading/disuse atrophy in a subject in need thereof. In some aspects, the methods can comprise administering to the subject a therapeutically effective amount of 15-hydroxyprostaglandin dehydrogenase (15-PGDH-I, SW033291), thereby treating unloading/disuse atrophy in the subject. In some aspects, the 15-PGDH-I can be administered orally, intravenously, intramuscular, intra-articularly, or subcutaneously. In some aspects, the subject in need thereof has unloading/disuse atrophy. In some aspects, the methods can further comprise identifying the subject in need of treatment before the administering step as having an increase in expression of at least one mitochondrion gene or a decrease in at least one biological process gene compared to a control subject without unloading/disuse atrophy. In some aspects, the at least one mitochondrion gene can be selected from the group of mitochondrially encoded cytochrome c oxidase III (MT-CO3), fumarylacetoacetate hydrolase domain-containing protein 1 (FAHD1), lon protease homolog (LONP1), phosphoenolpyruvate carboxykinase 2 (PCK2), glutaredoxin-1 (GLRX), RAB32, Bcl-2 related ovarian killer (BOK), cytochrome C oxidase assembly factor 4 homolog (COA4), dehydrogenase/reductase 4 (DHRS4), fatty acid desaturase 1 (FADS1), EF-hand domain family member D1 (EFHD1), NX4, MTHFD2, and serum/glucocorticoid regulated kinase 1 (SGK1). In some aspects, the at least one biological process gene can be selected from the group consisting of protocadherin gamma C3 (PDCHGC3), prostaglandin-endoperoxide synthase 1 (PTGS1), transforming growth factor beta induced (TGFB1), NYN domain and retroviral integrase containing (NYNRIN), neuroligin 2 (NLGN2), hemicentin-1 (HMCN1), dystonin (DST), zinc finger and BTB domain-containing 20 (ZBTB20), laminin alpha 2 (LAMA2), collagen type XII alpha 1 chain (COL12A1), proline rich coiled-coil 2C (PRRC2C), nuclear factor of activated T-cells 5 (NFAT5), golgin subfamily B member 1 (GOLGB1), SCUBE3, zinc finger protein 469 (ZNF469), heparan sulfate proteoglycan 2 (HSPG2), PH and SEC7 domain-containing protein 3 (PSD3), cluster of differentiation 109 (CD109), neuroblast differentiation-associated protein AHNAK (AHNAK), and neurogenic locus notch homolog protein 2 (NOTCH2). In some aspects, the methods can further comprise identifying the subject in need of treatment before the administering step as having a decrease in myotube length or myotube width compared to a control subject without unloading/disuse atrophy.

Disclosed herein are methods of reducing or ameliorating one or more symptoms of unloading/disuse atrophy in a subject in need thereof. In some aspects, the methods can comprise administering to the subject a therapeutically effective amount of 15-hydroxyprostaglandin dehydrogenase (15-PGDH-I, SW033291), thereby reducing or ameliorating one or more symptoms of unloading/disuse atrophy in the subject. In some aspects, the one or more symptoms of unloading/disuse atrophy can be decreased muscle mass, limb size discrepancies, numbness, weakness, tingling, decreased muscle strength and/or size, difficulty with movement, balance, coordination or a combination thereof, pain, a decrease in slow-twitch fibers, an increase in fast-twitch fibers, insulin resistance, or a combination thereof. In some aspects, the 15-PGDH-I can be administered orally, intravenously, intramuscular, intra-articularly, or subcutaneously. In some aspects, the subject in need thereof has unloading/disuse atrophy. In some aspects, the methods can further comprise identifying the subject in need of treatment before the administering step as having an increase in expression of at least one mitochondrion gene or a decrease in at least one biological process gene compared to a control subject without unloading/disuse atrophy. In some aspects, the at least one mitochondrion gene can be selected from the group of mitochondrially encoded cytochrome c oxidase III (MT-CO3), fumarylacetoacetate hydrolase domain-containing protein 1 (FAHD1), lon protease homolog (LONP1), phosphoenolpyruvate carboxykinase 2 (PCK2), glutaredoxin-1 (GLRX), RAB32, Bcl-2 related ovarian killer (BOK), cytochrome C oxidase assembly factor 4 homolog (COA4), dehydrogenase/reductase 4 (DHRS4), fatty acid desaturase 1 (FADS1), EF-hand domain family member D1 (EFHD1), NX4, MTHFD2, and serum/glucocorticoid regulated kinase 1 (SGK1). In some aspects, the at least one biological process gene can be selected from the group consisting of protocadherin gamma C3 (PDCHGC3), prostaglandin-endoperoxide synthase 1 (PTGS1), transforming growth factor beta induced (TGFB1), NYN domain and retroviral integrase containing (NYNRIN), neuroligin 2 (NLGN2),hemicentin-1 (HMCN1), dystonin (DST), zinc finger and BTB domain-containing 20 (ZBTB20), laminin alpha 2 (LAMA2), collagen type XII alpha 1 chain (COL12A1), proline rich coiled-coil 2C (PRRC2C), nuclear factor of activated T-cells 5 (NFAT5), golgin subfamily B member 1 (GOLGB1), SCUBE3, zinc finger protein 469 (ZNF469), heparan sulfate proteoglycan 2 (HSPG2), PH and SEC7 domain-containing protein 3 (PSD3), cluster of differentiation 109 (CD109), neuroblast differentiation-associated protein AHNAK (AHNAK), and neurogenic locus notch homolog protein 2 (NOTCH2). In some aspects, the methods can further comprise identifying the subject in need of treatment before the administering step as having a decrease in myotube length or myotube width compared to a control subject without unloading/disuse atrophy.

In some aspects, 15-PGDH-I can be formulated for oral or parental administration. In some aspects, the parenteral administration can be intravenous, subcutaneous, intramuscular or direct injection. In some aspects, the 15-PGDH-I can be formulated for oral, intramuscular, intravenous, subcutaneous, intrathecal, direct injection or intraperitoneal administration.

In some aspects, 15-PGDH-I can be formulated to include a therapeutically effective amount and be contained within a pharmaceutical formulation. In some aspects, the pharmaceutical formulation can be a unit dosage formulation. The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be any of the disclosed compounds described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

The therapeutically effective amount or dosage of 15-PGDH-I, used in the methods as disclosed herein, applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

In some aspects, the therapeutically effective amount of 15-PGDH-I can be administered orally or parentally. In some aspects, the therapeutically effective amount of the 15-PGDH-I can be administered orally or parentally. In some aspects, the parenteral administration can be intravenous, subcutaneous, intramuscular, or direct injection.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of 15-PGDH-I can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions (e.g., 15-PGDH-I) described herein can be administered in conjunction with other therapeutic modalities to a subject in need of therapy. The present compounds can be given to prior to, simultaneously with or after treatment with other agents or regimes. For example, 15-PGDH-I can be administered in conjunction with standard therapies used to treat unloading/disuse atrophy.

The dosage to be administered depends on many factors including, for example, the route of administration, the formulation, the severity of the patient's condition/disease, previous treatments, the patient's size, weight, surface area, age, and gender, other drugs being administered, and the overall general health of the patient including the presence or absence of other diseases, disorders or illnesses. Dosage levels can be adjusted using standard empirical methods for optimization known by one skilled in the art. Administrations of the compositions described herein can be single or multiple (e.g., 2- or 3-, 4-, 6-, 8-, 10-, 20-, 50-, 100-, 150-, or more fold). Further, encapsulation of the compositions in a suitable delivery vehicle (e.g., polymeric microparticles or implantable devices) can improve the efficiency of delivery.

Described herein are the findings that skeletal muscle constructs exposed to microgravity show impaired muscle regeneration and muscle atrophy. These muscle constructs can be engineered to be used in a screening assay to identify drugs that can increase myogenesis, and improve muscle regeneration and muscle atrophy. Further, these muscle constructs can be used as a model for sarcopenia and unloading/disuse atrophy.

### EXAMPLES

### Example 1

### Summary

Microgravity has been shown to lead to both muscle atrophy and impaired muscle regeneration. The purpose was to study the efficacy of microgravity to model impaired muscle regeneration in an engineered muscle platform and then to demonstrate the feasibility of performing drug screening in this model. Engineered human muscle was launched to the International Space Station National Laboratories, where the effect of microgravity exposure for 7 days was examined by transcriptomics and proteomics approaches. Gene set enrichment analysis of engineered muscle cultured in microgravity, compared to normal gravity conditions, highlighted a metabolic shift towards lipid and fatty acid metabolism, along with increased apoptotic gene expression. The addition of pro-regenerative drugs, insulin-like growth factor-1 (IGF-1) and a 15-hydroxyprostaglandin dehydrogenase inhibitor (15-PGDH-i), can partially inhibit the effects of microgravity. In summary, microgravity mimics aspects of impaired myogenesis, and the addition of these drugs could partially inhibit the effects induced by microgravity.

### Introduction

Representing approximately 40% of body weight, skeletal muscle is one of the most abundant tissues in the human body (Reid and Fielding, 2012). Skeletal muscle can regenerate itself through satellite cells, a reservoir of quiescent muscle stem cells that can be activated with injury or disease and give rise to newly formed multi-nucleated myotubes and myofibers (Mauro, 1961; Morgan and Partridge, 2003). As civilian space travel to the International Space Station and beyond will become increasingly more common in the next century, it is important to better understand how microgravity affects the regenerative capacity of muscle. Mice exposed to microgravity have shown reduced muscle regeneration, compared to in normal gravity, as demonstrated by relatively fewer postmitotic nuclei within myofibers as an indicator of impaired de novo muscle fiber formation (Radugina et al., 2018). Similarly, in vitro studies composed of murine myoblasts seeded within three-dimensional hydrogels in simulated microgravity also show reduced myogenesis, based on the formation of shorter and thinner myotubes with reduced fusion capacity (Ren et al., 2024). These data support a larger body of literature that microgravity exerts profound effects on skeletal muscle (Fitts et al., 2010; Juhl et al., 2021; Trappe, 2009).

The identification of countermeasures against the effects of microgravity on muscle regeneration is an increasing priority for continued space travel. Developing drugs that counteract the effects of microgravity is a promising strategy, but no FDA-approved drug currently exists, partially due to the limited access of participants to microgravity conditions. In contrast, a muscle-on-a-chip in microgravity platform can be performed in a facile manner in vitro and can allow for screening many drugs in parallel. Through the US National Science Foundation and the Center for the Advancement of Science in Space (“CASIS”), we were offered a unique opportunity to perform tissue engineering and mechanobiology research aboard the International Space Station National Laboratory (“ISSNL”). With the assistance of astronauts, we applied our expertise in the engineering of skeletal muscle (Huang et al., 2006; Nakayama et al., 2019) towards the study of muscle myogenesis using a muscle-on-a-chip platform. By performing multi-omics analyses, we show that the muscle-on-a-chip platform in microgravity can mimic salient aspects of impaired muscle regeneration after merely 7 days in microgravity. Furthermore, we demonstrate the feasibility of drug screening in this microgravity platform by using two drugs that can partially prevent the negative effects of microgravity in the muscle-on-a-chip bioconstructs.

### Results

### A. Engineering of a Muscle-On-a-Chip System in Microgravity

To adapt engineered muscle for microgravity conditions aboard the ISSNL, we developed a muscle-on-a-chip platform to immobilize the engineered muscle onto rigid hydrophobic glass for culture within a flight-compatible custom-made bioreactor. Skeletal muscle constructs composed of primary human myotubes on parallel-aligned collagen nanofibrils were generated by a facile shear-based collagen extrusion assay. Taking advantage of collagen fibrillogenesis being controlled by both pH and shear, we sheared acidic monomeric collagen type I from a blunt needle onto a rigid glass substrate bathed in pH neutral buffer (FIG. 1A), forming strips of collagen with highly organized nanofibrils. The collagen strips were composed of nanofibrils that were <100 nm in diameter, and the strips were then bundled together (FIGS. 1B, 1C). The anisotropic organization of the collagen fibrils can provide topographical patterning cues to induce primary human skeletal muscle cells to organize along the direction of the nanofibrils (FIG. 1D) and subsequently to form aligned myotubes (FIG. 1E). The cell-seeded scaffolds were denoted as a “muscle-on-a-chip.”

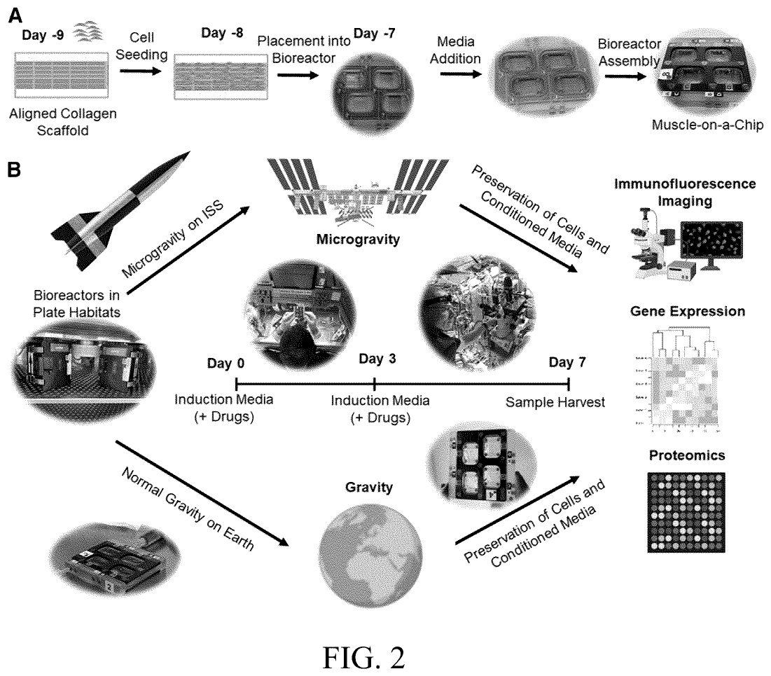

To adapt the muscle-on-a-chip platform to microgravity, microgravity-compatible bioreactors were custom-made by Bioserve Space Technologies. Prior to launch, the flight hardware was assembled by securing the muscle-on-a-chip bioconstructs within the chamber, filling the chamber with medium, and sealing with an air permeable membrane stabilized with a solid frame (FIGS. 2, 3A, 3B). During transit to the ISSNL on a Cygnus vehicle, the bioreactors were transported within gas-sealed space habitats that maintained temperature and carbon dioxide levels (FIGS. 2, 3C). Upon arrival at the ISSNL, the space habitats housing the bioreactors were placed into the Space Automated Bioproduct Lab incubator and maintained at 37° C. and 5% CO2. The experiments were initiated (day 0) in microgravity with the syringe-mediated replacement of the media with induction media that supports myotube formation (FIG. 2). Comparable bioreactors were assembled on Earth in normal gravity conditions, and the same experiments were performed in parallel to samples in microgravity.

### B. Microgravity Alters Myogenesis and Genetic Program in Skeletal Muscle-On-A-Chip

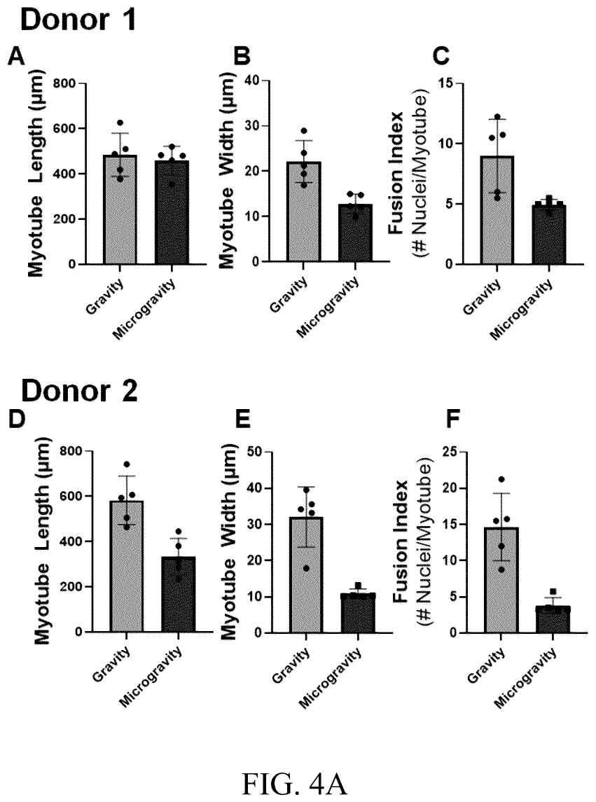

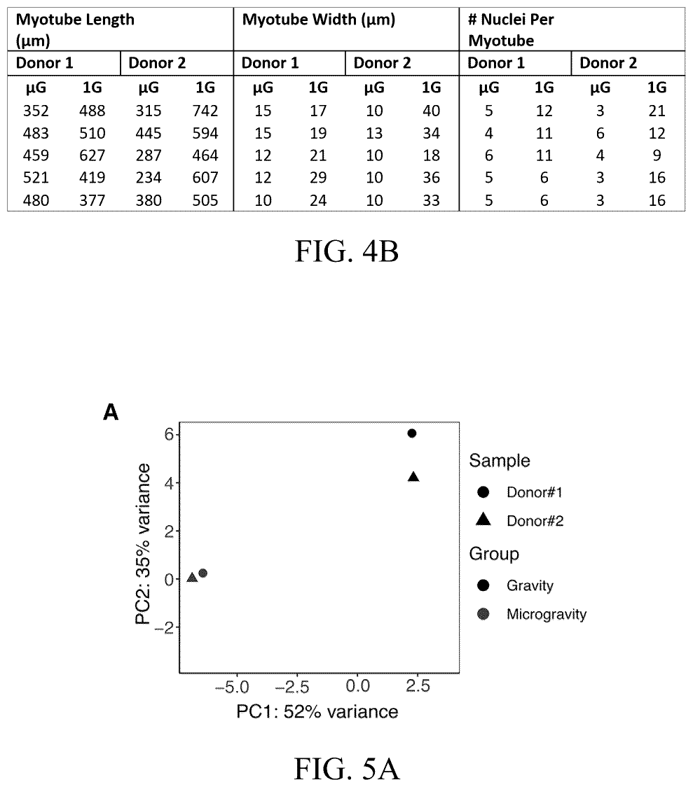

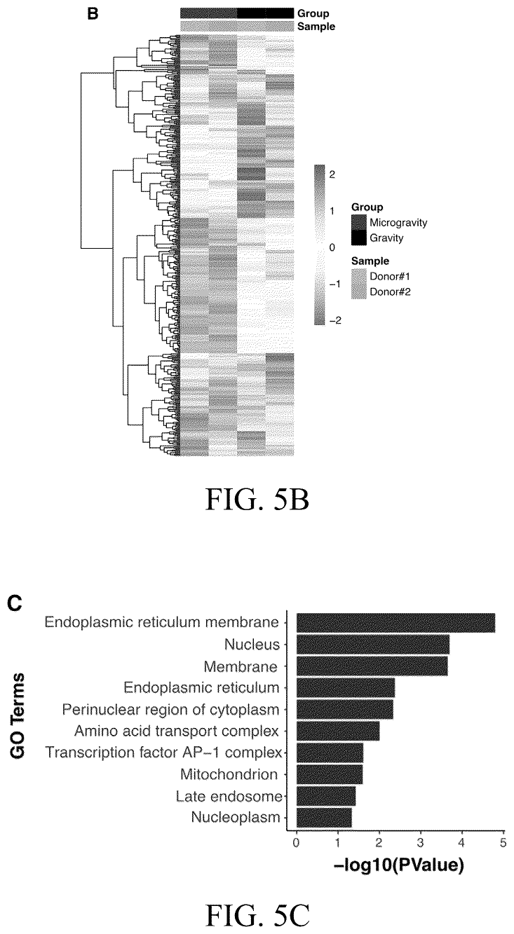

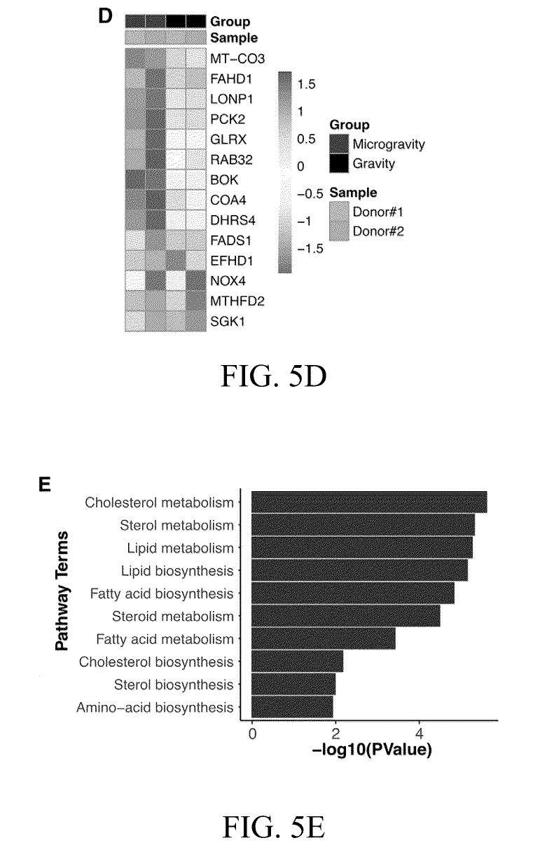

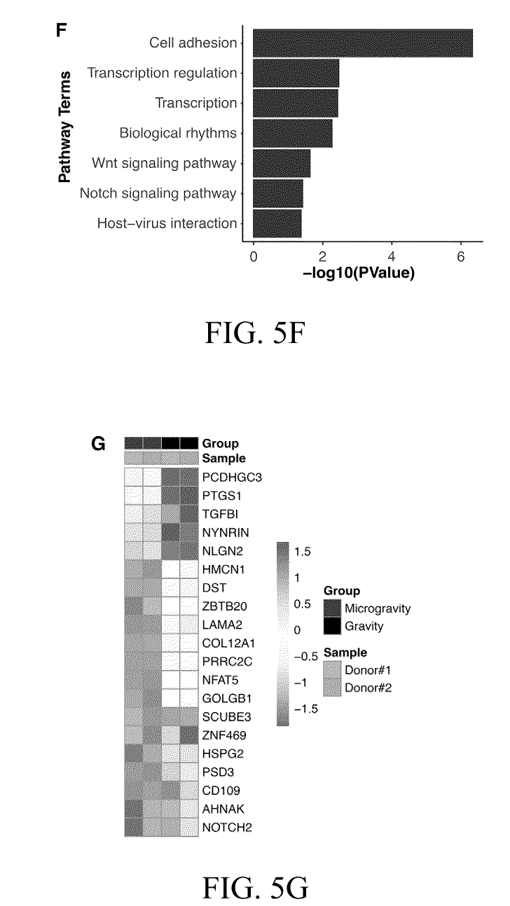

We first quantified myotube formation from healthy donor-derived engineered muscle after 7 days of exposure to microgravity. Our results showed reduced myotube length, width, and fusion index in microgravity, compared to gravity (FIGS. 4A, 4B). This finding of reduced myogenesis in microgravity was then corroborated by transcriptional analysis. Following 7 days of differentiation and myotube formation, the muscle-on-a-chip bioconstructs in both microgravity and in gravity from two healthy donors were assessed by RNA sequencing (“RNA-seq”). We detected 58,735 genes, of which 25,914 passed the threshold for expression. Transcripts of low count, expressed in less than two samples, were excluded. Samples were clustered by the gravity condition (FIG. 5A), where 107 genes were upregulated and 286 genes were downregulated in microgravity samples with a false-discovery rate (“FDR”)<10% (FIG. 5B). DAVID analysis classified 104 upregulated genes in microgravity into the Cellular Component Gene Ontology (“GO-CC”) term. The analysis shows significant enrichment in genes encoding proteins located in various subcellular structures (FIG. 5C). Among these genes, 14 of them were annotated under the “mitochondrion” GO-CC (FIG. 5D). For example, mitochondrially encoded cytochrome c oxidase III (MT-CO3) has previously been reported to increase in muscle stem cells during aging (Perez et al., 2022), which is associated with impaired regeneration. Since muscle has high metabolic function, proper mitochondrial biogenesis is necessary for regeneration.

Pathway enrichment analysis also supported changes in metabolic pathways, including genes implicated in cholesterol, lipid, and fatty acid metabolism (FIG. 5E). Among them, fatty acid desaturase 1 (FADS1) and mitochondrial matrix Lon peptidase 1 (LONP1), were also implicated in cellular responses to muscle regeneration (Huang et al., 2020). Additionally, the pro-apoptotic gene, BOK, along with cell survival and differentiation-associated genes, serum/glucocorticoid regulated kinase 1 (SGK1), and NADPH oxidase 4 (NOX4) (Dehner et al., 2008; Pedruzzi et al., 2004; Yakovlev et al., 2004), were upregulated in microgravity. SGK1 and NOX4 are associated with muscle mass maintenance and myoblast differentiation, respectively (Andres-Mateos et al., 2013; Youm et al., 2019). In particular, SGK1 can regulate muscle homeostasis by the downregulation of proteolysis and autophagy (Andres-Mateos et al., 2013). NOX4-derived reactive oxygen species (ROS) positively regulate myotube formation (Youm et al., 2019), which may be important for muscle regeneration.

Pathway analysis of decreased genes in microgravity highlights significant changes in biological processes, including genes involved in cell adhesion and cell fate decision pathways, including Wnt and Notch signaling pathway (FIG. 5F). The top 20 downregulated genes included those involved in cell adhesion (protocadherin gamma C3 [PCDHGC3], transforming growth factor β Iiduced [TGFBI], neuroligin 2 (NLGN2), dystonin (DST), laminin α2 [LAMA2], collagen type XII alpha 1 chain [COL12A1]), Notch signaling pathway (NOTCH2), and inflammation (prostaglandin-endoperoxide synthase 1 (PTGS1/COX1)) (FIG. 5G). In particular, NOTCH2, which is an important marker of muscle stem cell self-renewal and stem cell fate decision (Yartseva et al., 2020), was significantly downregulated in microgravity. Downregulation of NOTCH2 gene expression in satellite cells leads to a decrease in their ability to differentiate (Fujimaki et al., 2018), suggesting an impairment in the balance between proliferation and differentiation of primary human skeletal muscle cells in microgravity. The downregulation of NOTCH2 concurs with our in vitro findings of a significantly lower fusion index in the engineered muscles in microgravity (FIG. 4A). Additionally, ECM remodeling is associated with muscle regeneration (Calve et al., 2010), and we observed significant downregulation of ECMs such as COL12A1, LAMA2, and heparan sulfate proteoglycan 2 (HSPG2) genes. Together, this analysis suggested that microgravity conditions can induce a cellular phenotype that is associated with hallmarks of impaired regeneration, including metabolic remodeling, imbalance between proliferation, differentiation, and decreased cell-cell and cell-ECM adhesion.

To further examine the effects of microgravity, we performed proteomic analysis of 200 proteins from the conditioned media of bioconstructs cultured in microgravity or gravity conditions for 7 days. In comparing the microgravity versus gravity samples with FDR<25%, we identified 5 proteins that were secreted in greater abundance and 4 proteins that were of reduced abundance. The proteins of greater abundance in microgravity include Eotaxin-3 (CCL26), C-X-C motif chemokine ligand 16 (CXCL16), growth differentiation factor 15 (GDF-15), tumor necrosis factor superfamily member 14 (LIGHT/TNSF14), and pupoid fetus (PF). Of these proteins, CCL26 and CXCL16 are chemoattractants known to induce immune cell infiltration and are associated with chronic inflammation (Scholz et al., 2007; Yamada et al., 2019). In addition, GDF-15 is a biomarker for mitochondrial dysfunction and cellular senescence (Fujita et al., 2016). The proteins of reduced abundance in microgravity include bone morphogenetic protein 4 (BMP-4), Resistin, C-X-C motif chemokine ligand 12 (CXCL12/SDF-1b), interleukin-16 (IL-16) and CD40. CXCL12, in particular, is an important player in the maintenance of muscle and myogenesis (Gilbert et al., 2019; Puchert et al., 2016).

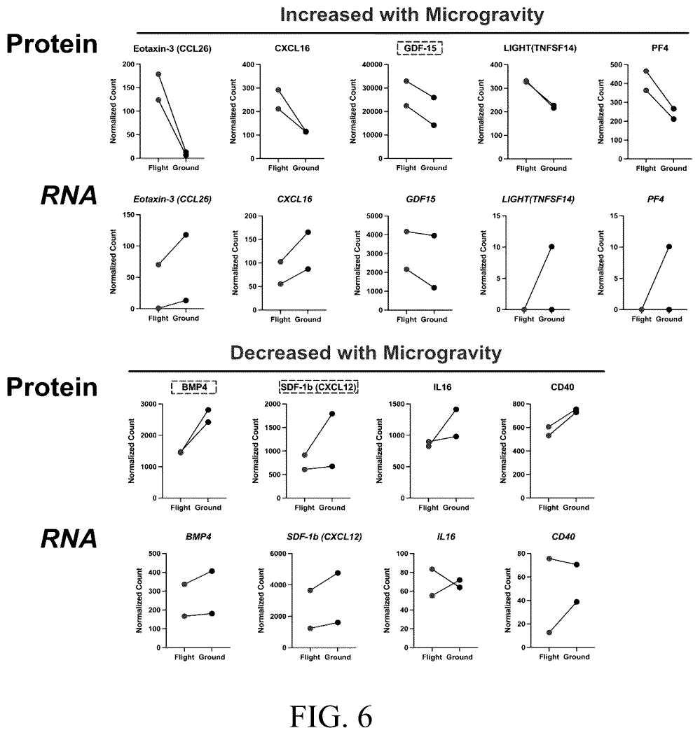

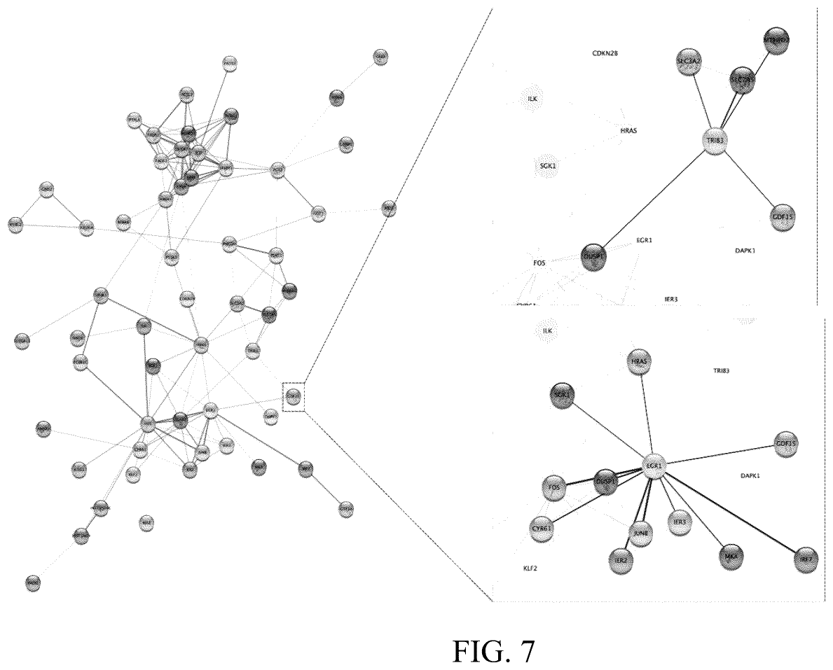

To determine whether the secreted proteomes were related to their RNA expression levels, we compared their relative expression between microgravity and gravity samples. We observed that some differential protein changes can be generally correlated with their corresponding RNA expression level changes (FIG. 6). For example, GDF-15 was consistently increased in microgravity conditions at both the protein and RNA levels, whereas BMP4 and SDF-1b (CXCL12) levels were consistently downregulated in microgravity at both the protein and RNA levels (FIG. 6). Since GDF-15 has previously been reported as a diagnostic biomarker for conditions associated with impaired muscle regeneration such as aging (Ito et al., 2018; Kim et al., 2020), we searched for potential interacting genes of GDF-15 in microgravity samples. The protein-protein interaction (“PPI”) network was constructed by STRING database using 107 genes that were upregulated with microgravity, including GDF-15. The unconnected genes were then removed. The resulting PPI network contained 57 nodes and 135 edges (FIG. 7), where a node represents a gene and an edge represents the relationship between the two inter-connected genes. From this analysis, we identified early growth response factor 1 (EGR1) and tribbles homologue 3 (TRIB3) as the first neighbors of GDF-15. These results suggest that GDF-15 and its immediate neighbors may play an important role in microgravity-induced impairment in myogenesis

### C. Microgravity Partially Mimics Genomic Features of Impaired Muscle Regeneration in Sarcopenia

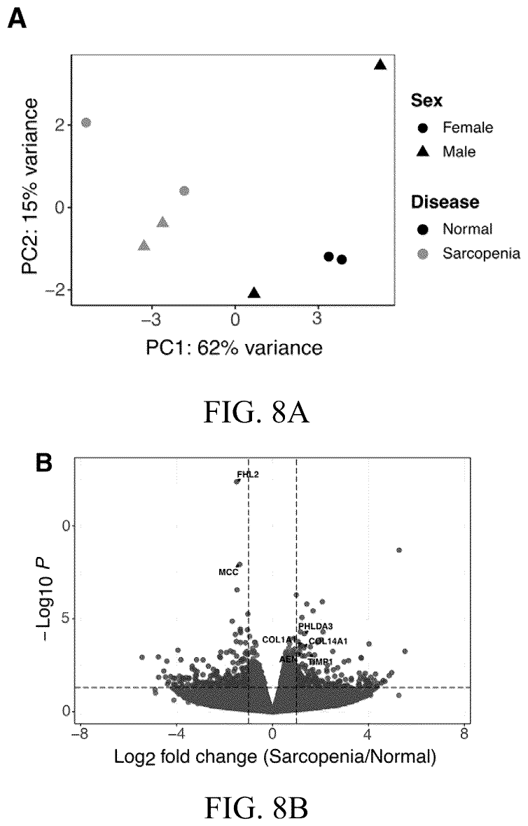

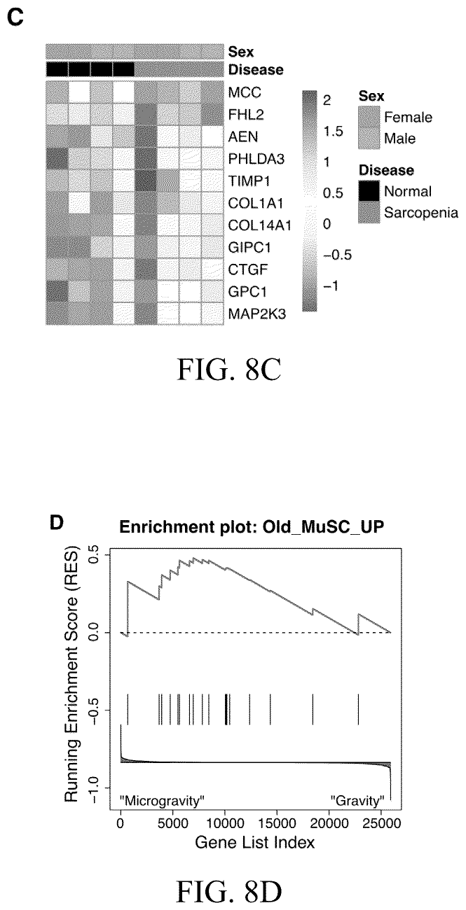

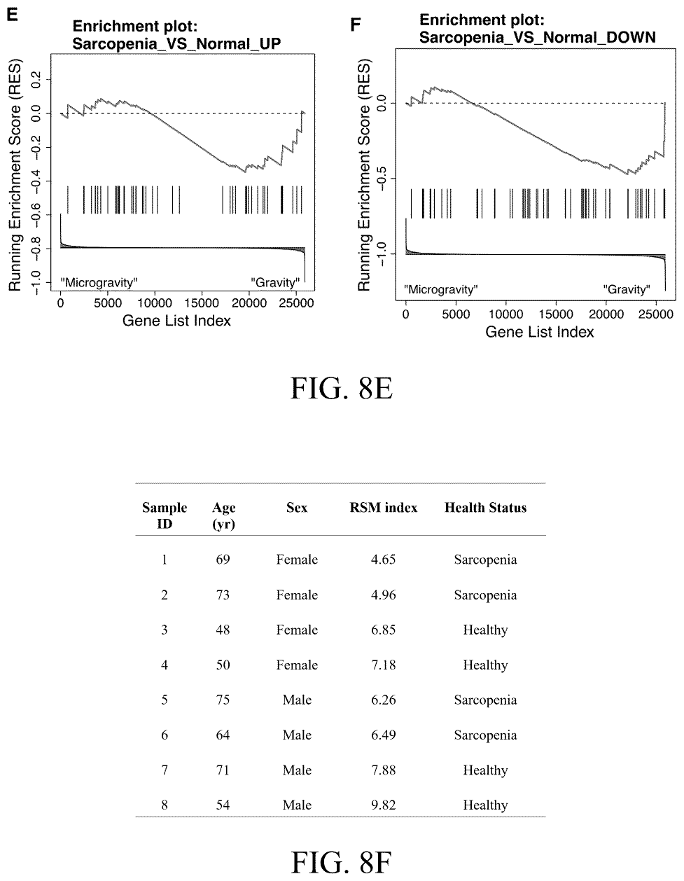

Since impaired muscle regeneration is associated with clinical conditions such as sarcopenia, we assessed whether the muscle-on-a-chip in microgravity platform mimics regeneration-related signaling pathways in clinical sarcopenia muscle samples. We compared the transcriptome of human muscle tissue obtained from either healthy donors (denoted as “Normal”; (n=4)), or those with sarcopenia, as defined by accepted cutoffs for the RSM index (n=4) (FIGS. 8A, 8F). RNA-seq detected 58,735 genes, of which 29,313 passed the threshold for expression. In particular, 48 genes were upregulated and 113 genes were downregulated in sarcopenia samples with an FDR<25% (FIG. 8B). Among the top upregulated genes were those involved in apoptotic signaling pathways in response to DNA damage (AEN, PHLDA3), ECM organization (TIMP1, COL1A1, COL14A1) and the VEGFA/VEGFR2 signaling pathway (GIPC1, CTGF, GPC1, and MAP2K3). Interestingly, more genes were downregulated in sarcopenia and they included genes that are implicated in Wnt signaling (MCC) and associated with rhabdomyosarcoma (FHL2) (FIG. 8C). Importantly, an increase in apoptosis or impairment in skeletal muscle cell function could contribute to the development or progression of sarcopenia (Chung and Ng, 2006; Leeuwenburgh, 2003; Siu et al., 2005), in which the upregulation of markers such as AEN and PHLDA3 are implicated in p53-dependent apoptosis (Cruz-Jentoft and Sayer, 2019; Kawase et al., 2008; Kawase et al., 2009).

We next established a custom gene set library to assess whether engineered muscle in microgravity mimics features of sarcopenia from clinical samples. In brief, we used the up- and down-regulated gene list from sarcopenia versus normal samples and converted the list into a GSEA analysis-compatible format. We also analyzed publicly available datasets derived from old adult muscle myoblasts (GSE52699) and generated an enriched gene set list compared to young myoblasts. As shown in FIG. 8D, custom GSEA results indicate that genes upregulated in old human myoblasts (“Old_MuSC_UP” gene set) were over-represented in the muscle-on-a-chip bioconstruct in microgravity, as indicated by the positive running enrichment score (“RES”). Although some of the sarcopenia-related genes were significantly decreased in gravity, compared to microgravity conditions, the enrichment plot for Sarcopenia_vs_Normal_UP shown in FIG. 8E suggests that the genes upregulated by sarcopenia were not enriched by microgravity. However, the genes downregulated by sarcopenia were also downregulated with microgravity exposure (FIG. 8E). These custom GSEA results indicate that the muscle-on-a-chip bioconstructs in microgravity had a negative enrichment of genes that were downregulated in the “Sarcopenia_VS_Normal_DOWN” gene set (FIG. 8E). These data reveal a similarity in the transcriptional signature between the clinical sarcopenia samples and engineered muscle in microgravity samples, in which the genes that were downregulated in sarcopenia muscle tissue were also downregulated in the engineered muscle in microgravity conditions, compared to that of the gravity condition (FIG. 8E). Conversely, the genes that were upregulated in sarcopenia were not enriched in microgravity condition compared to that of gravity conditions (FIG. 8E). Overall, the custom GSEA results suggest that space flight mimics in part the genomic signature of old adult myoblasts and sarcopenia.

### D. Drug Treatment Partially Prevents Adverse Effects of Microgravity

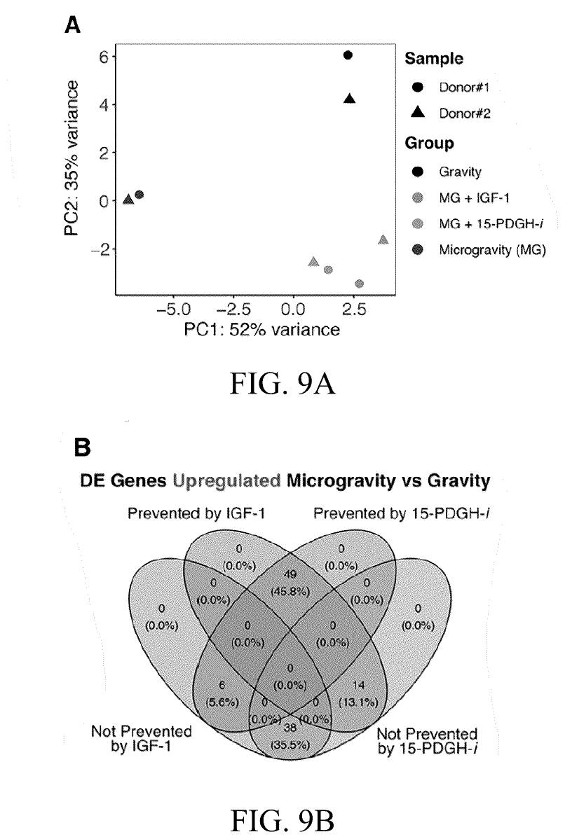

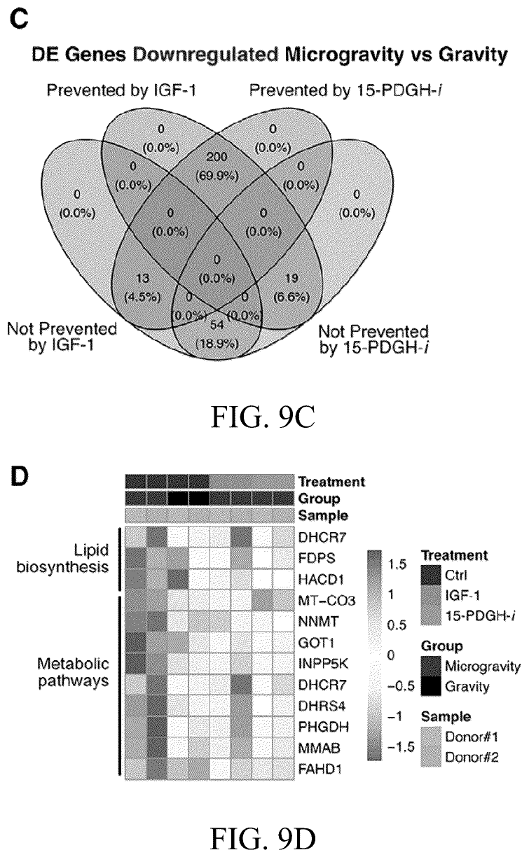

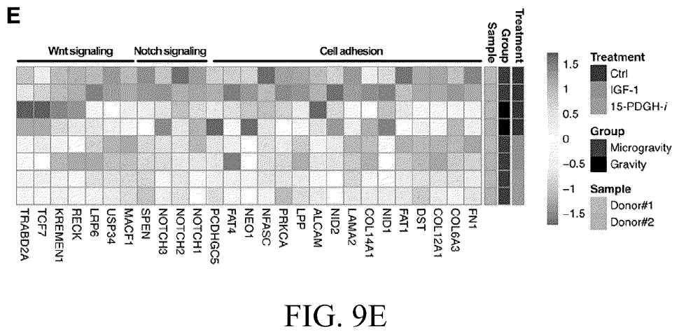

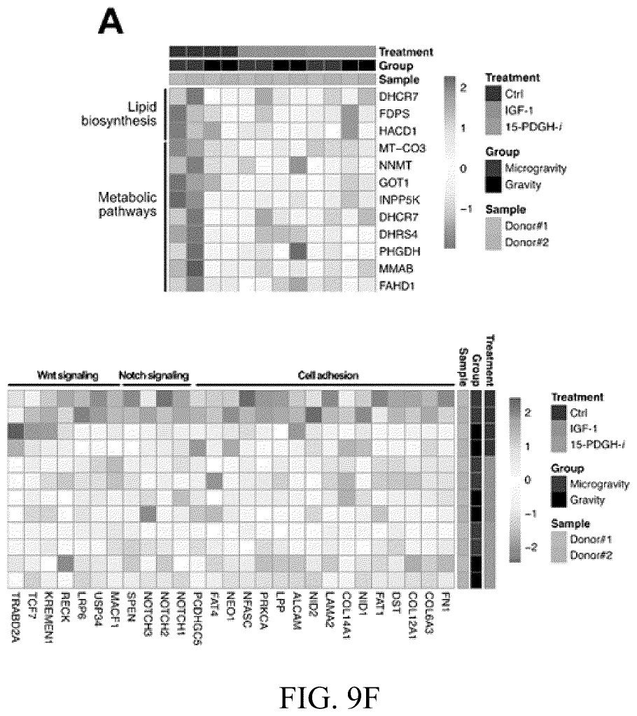

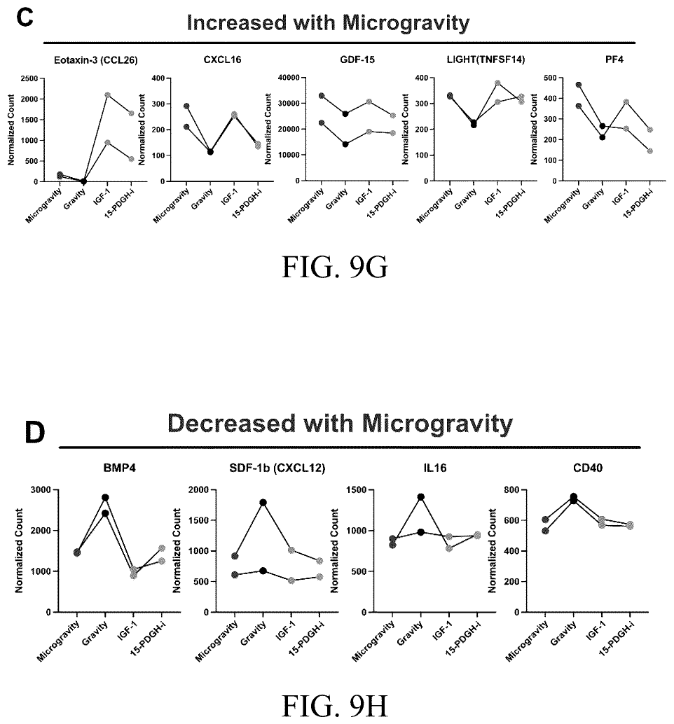

We next used the muscle-on-a-chip platform to perform proof-of-concept drug screening studies. Recently published studies showed that a 15-PDGH inhibitor (15-PDGH-i) could stimulate myogenesis as well as abrogate sarcopenia in preclinical models through the regulation of prostaglandin E2 signaling (Ho et al., 2017; Palla et al., 2021). Besides 15-PDGH-i, we also examined insulin-like growth factor-1 (IGF-1) in the muscle-on-a-chip platform. The effects of IGF-1 in promoting muscle myogenesis and inhibition of muscle atrophy are well-established (Alcazar et al., 2020; Ascenzi et al., 2019; Ma et al., 2009; Machida and Booth, 2004; Yu et al., 2015). To examine the potential use of these two drugs to regulate myogenesis, the engineered skeletal muscle-on-a-chip bioconstructs were treated with either IGF-1 or 15-PDGH-i for 7 days in microgravity conditions, followed by RNA-seq analysis. Through principal component analysis, we found that, in microgravity, the drug-treated groups were generally more transcriptionally similar to the samples in gravity, rather than their microgravity counterparts (FIG. 9A). Out of 107 genes that were upregulated in microgravity compared to gravity counterparts, 49 genes showed similar expression levels to those of gravity conditions with IGF-1 and 15-PDGH-i treatment, suggesting that the drugs could prevent the transcriptional upregulation effects of microgravity. In addition, IGF-1 and 15-PDGH-i treatment prevented the transcriptional upregulation effects of microgravity in 6 and 14 genes, respectively (FIG. 9B).

Of the 286 downregulated genes with microgravity, 200 genes showed similar expression levels to those of gravity conditions with IGF-1 and 15-PDGH-i treatment, suggesting that the drugs could prevent the transcriptional downregulation effects of microgravity. In particular, IGF-1 and 15-PDGH-i treatment prevented the transcriptional downregulation effects of microgravity of 13 genes and 19 genes, respectively (FIG. 9C). As noted earlier, pathway enrichment analysis highlighted an enrichment of genes associated with metabolic pathways including lipid and fatty acid metabolism in microgravity samples. We found that the drug-treatment prevented a shift in metabolic profiles and maintained normal expression of genes involved in lipid biosynthesis (DHCR7, FDPS, HACD1) and metabolic pathways (MT-CO3, NNMT, GOT1, INPP5K, DHCR7, DHRS4, PHGDH, MMAB, FAHD1) (FIG. 9D). Moreover, we also observed that many genes implicated in cell adhesion, the Notch signaling pathway (NOTCH1, NOTCH2, NOTCH3, SPEN), and the Wnt signaling pathway (MACF1, USP34, LRP6, RECK, KREMEN1, TCF7, TRABD2A) were downregulated with space flight, but were similar in expression level to gravity samples upon drug treatment (FIGS. 9E, 9F). Overall, the drug-treated samples displayed a signature that more closely resembled gravity samples, compared to their microgravity counterparts.

Since the addition of IGF-1 and 15-PDGH-i prevented some effects of microgravity at the transcriptional level, we further assessed their effect on proteins found in conditioned media at 7 days after drug treatment. Interestingly, we found that IGF-1 and 15-PDGH-i treatment can prevent the ability of microgravity to increase GDF-15 protein levels (FIG. 9G). Additionally, 15-PDGH-i treatment can protect CXCL16 and PF4 protein levels from rising to levels, similar to those of microgravity samples. Despite the ability of the drugs to prevent an increase in protein levels to reach those of microgravity samples, none of the proteins that were decreased in microgravity compared to gravity conditions could be normalized to the levels associated with gravity samples by the addition of drugs (FIG. 9H). We further tested whether the first neighbors of GDF-15 (EGR1, and TRIB3) are affected with drug treatment. As described earlier, EGR1 was not affected by drug treatment, whereas IGF-1 addition prevented TRIB3 level from reaching that of microgravity samples (FIG. 9I). Taken together, our multi-omics analysis demonstrates that microgravity induces in engineered muscle a signature that reflects salient aspects of impaired myogenesis, which could be partially prevented by IGF-1 or 15-PDGH-i.

### Discussion

The salient findings of work are: 1) an engineered muscle-on-a-chip platform can facilitate the study of microgravity effects on myotube formation and muscle-related biological processes; 2) microgravity can mimic salient aspects of impaired regeneration, based on RNA sequencing and proteomic analysis; 3) microgravity effects on engineered muscle can be prevented in part by IGF-1 and 15-PDGH-i; and 4) drug screening in microgravity using an engineered muscle-on-a-chip platform is feasible. These findings suggest that aspects of impaired muscle myogenesis can be modeled in just one week of exposure in microgravity, and that drug screening in microgravity may be a useful platform for identifying potential drug targets that regulate myogenesis.

The study of the effects of microgravity on engineered muscle-on-a-chip is an emerging area of research. Our findings using engineered muscle in microgravity concur with Vandenburgh et al., whose engineered avian muscle aboard the Space Transportation Shuttle for 12 days showed evidence of reduced myofiber size and cross-sectional area (Vandenburgh et al., 1999). Additionally, a larger body of work derived from in vitro cultured myoblasts in microgravity or simulated microgravity further support the finding that microgravity can abrogate myogenic differentiation and myotube size, owing to cellular senescence and epigenetic modifications (Furukawa et al., 2018; Parafati et al., 2023; Takahashi et al., 2021).

Previous studies have reported multiple effects of spaceflight on human physiology. Most recently, large-scale multi-omics, systems biology analytical approaches profiled 59 astronauts from NASA's GeneLab to determine transcriptomic, proteomic, metabolomic, and epigenetic responses to spaceflight, which highlighted a significant enrichment for mitochondrial processes and DNA damage (da Silveira et al., 2020). Alterations in mitochondrial activity and impaired lipid metabolism are associated with muscle atrophy (Nikawa et al., 2004; Vitry et al., 2022). In line with these reports, our engineered muscle-on-a-chip platform exposed to microgravity for 7 days also demonstrated changes in mitochondrial genes (FIG. 5C), a shift in metabolism towards lipid and fatty acid biosynthesis (FIG. 5E), and an increase in apoptotic processes (BOK, SGK1 and NOX4). The existence of mitochondrial dysfunction was further supported by proteomic studies reporting an increase in GDF-15. GDF-15 is a biomarker of dysfunctional mitochondria and cellular senescence (Fujita et al., 2016). Increased GDF-15 levels are associated with muscle atrophy in cancer models, and GDF-15 treatment was shown to induce muscle atrophy in C2C12 myotubes (Zhang et al., 2022). GDF-15 neutralization could restore muscle mass and function (Kim-Muller et al., 2023). More importantly, the addition of pro-regenerative drugs such as IGF-1 and 15-PGDH-i could partially prevent the effects of microgravity on our engineered muscle-on-a-chip platform.

Notch signaling is major regulator of myogenesis (Luo et al., 2005). The disruption in Notch signaling in aging muscle compromises new myofiber formation and impairs tissue regeneration as a potential contributing factor to sarcopenia (Arthur and Cooley, 2012; Carlson et al., 2008; Conboy et al., 2003). Notch inhibition also contributes to the dysregulation of cellular quiescence and can perturb the quiescent state of the muscle stem cells (Bjornson et al., 2012; van Velthoven and Rando, 2019). Notch signaling can also regulate the stability of a tumor suppressor, p53. The Notch-p53 axis declines with age and is detrimental to cell survival and expansion (Liu et al., 2018). Our data is consistent with the literature in demonstrating a downregulation in Notch signaling in microgravity (FIG. 5F). The loss of such a signal is correlated with reduced differentiation potential, as demonstrated by the impairment in myotube formation.

More genes were downregulated than upregulated in both microgravity versus gravity, as well as in the comparison of sarcopenia versus normal muscle samples. Microgravity samples were more sarcopenia-like in terms of genes that are important for apoptosis and Wnt signaling. The genes involved in apoptosis were upregulated, whereas the genes involved in Wnt signaling were downregulated in both microgravity and sarcopenia samples, respectively. Although the cellular response to gamma radiation and apoptosis processes increased with microgravity but were not prevented with IGF-1 or 15-PGDH-i treatment, the gene expression of Wnt signaling components including Lrp6 were prevented with drug treatment (FIG. 9E). Lrp6 is one of the two co-receptors involved in canonical Wnt signaling in vertebrates and conditional Lrp6 knockout reduced fiber size in mice (Gessler et al., 2022; Ren et al., 2021), highlighting its importance in muscle mass maintenance. Interestingly, microgravity increased the expression level of TRIB3, which negatively regulates cell survival and inhibits myogenic differentiation (Kato and Du, 2007; Saleem and Biswas, 2017). TRIB3 was identified in the PPI network as one of the two immediate neighboring gene of GDF-15. IGF-1 treatment can prevent the elevation of TRIB3 gene expression in microgravity. In a mouse model, TRIB3 is implicated in both age-related and food deprivation-induced muscle fiber atrophy and fibrosis (Choi et al., 2019; Shang et al., 2020). Furthermore, our findings align with a recent report that also demonstrated differential expression of TRIB3 and PCK2 under microgravity conditions (Parafati et al., 2023), further supporting the impact of microgravity on myogenesis and metabolism.

We acknowledge the limitations associated with this study. Owing to constraints associated with space research, only one launch was possible. For this reason, the data was derived from two primary human muscle donors as a measure of variance in the dataset. Furthermore, we acknowledge that measurements of muscle contractility could be informative, but such a functional assay would be most appropriate for engineered muscles that are more mature, which would have further increased the duration of the studies. Finally, the findings from the data in microgravity do not preclude the possibility of other contributing factors such as ionizing radiation. Despite these limitations, we believe that the observed effects of microgravity on engineered muscle are compelling and provide an avenue for future drug screening explorations in space for identifying drug targets that can enhance muscle myogenesis.

In conclusion, we show that engineered muscle-on-a-chip bioconstructs exposed to microgravity can induce prominent changes to their transcriptome that mimics aspects of impaired myogenesis. Gene set enrichment analysis demonstrated a shift in metabolic pathways towards lipid metabolism, along with downregulated Notch signaling pathways and the increased expression of apoptotic genes. Importantly, microgravity exposure to engineered muscle-on-a-chip bioconstructs resembled some similar features to that of clinical sarcopenia muscle. The effects of microgravity could be partly prevented by the addition of IGF-1 or a 15-PGDH-i. Together, this transcriptomic and proteomics analyses of microgravity effects using a muscle-on-a-chip platform demonstrate that microgravity mimics aspects of impaired myogenesis. This work further highlights the utility of microgravity as a unique environment for drug discovery.

### Experimental Procedures

### A. Materials Availability

This study did not generate new unique reagents.

### B. Data and Code Availability

The RNA Sequencing raw data files can be accessed from the Gene Expression Omnibus repository (GSE238215). All data may be made available by request to the corresponding author.

### C. Cell Culture

Primary human skeletal muscle cells (Cell Applications) from two individual donors (37-41 years old) were expanded in growth media consisting of Dulbecco's modified Eagle's medium (DMEM) containing 20% fetal bovine serum (FBS) and 1% penicillin/streptomycin at 37° C. and 5% CO₂. The primary cells were used within one passage of thawing.

### D. Fabrication of Aligned Nanofibrillar Collagen Strips

Preparation of the parallel-aligned collagen scaffold was prepared using a shear-based extrusion method, as described in our previous publications (FIG. 1A) (Alcazar et al., 2020; Hu et al., 2022; Nakayama et al., 2018; Nakayama et al., 2019; Nakayama et al., 2016). Briefly, rat-tail monomeric collagen type I (10 mg/ml in 0.02 N in acetic acid, pH 3.5, Corning) was dialyzed using polyethylene glycol (PEG, Sigma) flakes. Every 15 minutes, the moistened PEG flakes from the surface of the dialysis tubing were removed and replaced with dry PEG flakes and then returned to 4° C. After 30 minutes, the tubing was placed in a container with MilliQ water, and the moistened PEG was removed from the tubing surface. The dialyzed collagen was spun down at 3000 rpm for 1 min to remove air bubbles. Meanwhile, glass slides were submerged inside a Petri dish at 37° C. containing PBS (10×, pH 7.4). Dialyzed monomeric collagen was loaded into a 1-ml syringe with a 22 G blunt needle. Thin strips of collagen were extruded onto the glass slides using a steady sideways motion (FIG. 1A). Extrusion directly into a pH neutral buffer induced fibrillogenesis of the collagen fibrils along the direction of extrusion (FIG. 1B). The collagen strips were carefully removed from the glass slide, and then 8 strips were adhered as a bundle onto a hydrophobic glass chip (˜25×15 mm, Schott H, Nexterion). The collagen strips on the glass chips were dried for 2 hours at room temperature until salt crystals started to cover 50%-90% of the surface area. Subsequently, the scaffolds were washed with PBS (1×) and then dried for 24 hours before use. Routine scanning electron microscopy was performed based on previous studies (Huang et al., 2013).

### E. Generation of Muscle-on-a-Chip Bioconstructs and Assembly of Bioreactor

The parallel-aligned nanofibrillar collagen scaffolds immobilized on glass chips were disinfected by soaking in 70% ethanol for 10 minutes, air dried, and then rinsed in PBS (1×) three times before placing into a 6-well plate. Primary human skeletal muscle cells (˜2×105 cells, Cell Applications) from two donors were seeded onto each glass chip and incubated for 24 hours in growth medium. The cell-seeded glass chip is denoted as a “muscle-on-a-chip.” The muscle-on-a-chip from each donor was transferred to one chamber of a custom-built 4-chamber bioreactor (BioCell, Bioserve Space Technologies) and filled with growth media (FIGS. 2A, 3A, 3B). Each tissue chip was immobilized to the bottom of the bioreactor using a stabilizing bridge and overlayed with medium (FIG. 3A). Once tissue chips were in place, the bioreactor assembly was completed using a transparent oxygen-permeable fluorinated ethylene propylene and bioreactor lid, removing air bubbles, and rendering a sealed environment.

### F. Flight Experiment in Microgravity

During transit to the International Space Station National Laboratories (ISSNL) on the NASA Commercial Resupply Mission (NG-16) Cygnus spacecraft, the bioreactors containing muscle-on-a-chip bioconstructs were housed inside space habitats (FIGS. 2, 3C) that maintained ˜37° C. and 5% CO₂ for 7 days without changing their medium. After docking to the ISSNL, the bioreactors were transferred by space astronauts (or flight crew) to the Space Automated Bioproduct Lab for active maintenance of 37° C. and 5% CO₂ conditions. The media was exchanged in each chamber by needle/syringe access through designated inset and outlet ports in the bioreactor (FIG. 3D) in the controlled environment of the life sciences glovebox (FIG. 3E).

The experiments were initiated in microgravity with the exchange of media into an induction media consisting of DMEM supplemented with 2% horse serum and 1% penicillin/streptomycin (day 0) (FIG. 2). The media was exchanged again on day 3. The viability of samples was qualitatively monitored using an on-orbit phase contrast microscope (FIG. 3F). On day 7, the conditioned media within the bioreactors were harvested at stored at −80° C. The muscle-on-a-chip devices within each chamber were treated with either RNA Later stabilization agent (Fisher Scientific) and then stored at −80° C., or were fixed in 4% paraformaldehyde (Fisher Scientific) for 15 minutes and then stored at 4° C. Samples were maintained on the ISSNL at the indicated temperatures and then returned to Earth on Space X Cargo Resupply Service 23 about two months later. Semi-synchronous ground control experiments were performed on a separate set of muscle-on-a-chip bioconstructs from the same donors in identical bioreactors and syringe media exchange procedures. The bioreactors in normal gravity conditions were cultured in conventional incubators but were handled otherwise in the same manner and with the same time stamps, as in microgravity.

### G. Immunofluorescence Staining of Myotube Formation

Immunofluorescence staining was carried out on fixed cells by permeabilization on 0.1% Triton-X-100, followed by blocking in 1% bovine serum albumin (BSA, Sigma). The primary antibody consisted of skeletal muscle myosin heavy chain (Sigma) at 4° C. for 16 hours, followed by incubation in Alexafluor-488-conjugated or Alexafluor-594-conjugated secondary antibody (Fisher Scientific). Total nuclei were visualized by Hoechst 33342 dye (Fisher Scientific). Images of myotube formation in C2C12 myoblasts were acquired using a fluorescence microscope using 10× objectives (BZ-X710, Keyence). For quantification of primary human myotube formation within the bioconstructs after 7 days in microgravity or gravity conditions, images were acquired by confocal microscopy using 10× objectives (Zeiss, LSM-710). In particular, five z-stacks were acquired for each donor engineered muscle sample, and the maximum projection images were taken. Myotube lengths and widths in each image were quantified by ImageJ software. The fusion index was quantified based on the average number of nuclei per myotube.

### H. Sarcopenia Clinical Samples

As a basis for comparison, we further compared the transcriptomes of microgravity samples to that of banked human vastus lateralis muscle total RNA samples. Total RNA from 4 healthy and 4 sarcopenia donors (2 males and 2 females per group) were obtained from University of Kentucky Center for Muscle Biology (FIG. 8F) with approval from the Institutional Review Board (Protocol #46746). Samples were considered to have sarcopenia based on cutoffs for the relative skeletal muscle (“RSM”) index, which is defined as muscle mass normalized to stature squared (kg/m2) (Baumgartner et al., 1998). Females with RSM<5.46 kg/m² were considered to have sarcopenia, whereas RSM>5.46 kg/m² were considered to be healthy (Baumgartner, 2000; Kyle et al., 2001). Males with RSM<7.26 kg/m² were considered to have sarcopenia, whereas males with RSM>7.26 kg/m² were considered to be healthy (Baumgartner, 2000; Kyle et al., 2001).

### I. Bulk RNA Sequencing and Analysis

Upon sample return to Earth, the muscle-on-a-chip bioconstructs that had been stabilized in RNA were dissociated using TrypLE enzyme (Fisher Scientific) for total RNA and total DNA isolation (All Prep Mini Kit, Qiagen). Total RNA samples showing good RNA integrity by Agilent Bioanalyzer analysis were processed for library preparation and bulk RNA sequencing by Novogene Corporation. In brief, mRNA was purified from total RNA using poly-T oligo-attached magnetic beads. After fragmentation, the first strand cDNA was synthesized using random hexamer primers, followed by the second strand cDNA synthesis. Non-directional library preparation was completed after end repair, A-tailing, adapter ligation, size selection, amplification, and purification. The library was validated using Qubit assay (Fisher Scientific) and quantitative PCR, along with Agilent bioanalyzer analysis of size distribution detection. The libraries were pooled and sequenced using an Illumina NovaSeq 6000 S4 platform. The paired-end reads were then generated.

The raw data were processed as follows. Raw reads were pre-processed to remove reads containing adapter, poly-N, and low-quality reads. Trimmed reads were aligned to the human reference genome (hg38) using Hisat2 (v2.0.5) and featureCounts v1.5.0-p3 was used to generate count reads mapped to each gene. All the downstream analyses were performed on the clean data with high quality, using RStudio software v4.0.4. DESeq2 package v1.30.1 was used for differential gene expression analysis. Statistical analysis was performed using the Wald test statistic, and the resulting p values were adjusted using the Benjaminin and Hochberg's approach for controlling the false discovery rate (“FDR”). We analyzed microgravity versus gravity (control) bulk RNA sequencing transcriptomics at FDR<0.1 and sarcopenia versus normal at FDR<0.25 to assign genes as differentially expressed between the two groups. The raw data files can be accessed from the Gene Expression Omnibus repository (GSE238215).

Gene Ontology (GO) enrichment and pathway enrichment analysis of significantly differentially expressed genes was performed using DAVID (https://david.ncifcrf.gov/) with default settings. We used P<0.05 as significant GO and pathway enrichment.

### J. Custom GSEA Analysis

We computed custom GSEA results for sarcopenia and aging using GSEAplot package v0.1.0 (https://github.com/kelsiereinaltt/GSEAplot) with default settings. In brief, to assess the enrichment of genes associated with sarcopenia or old muscle stem cells, we generated the differentially expressed genes comparing sarcopenia versus normal human muscle tissue. Similarly, we generated differentially expressed genes from a published dataset comparing in vitro cultured old versus young muscle stem cells (Old_MuSC; GSE52699). The expression data were created using create_geneset_db function, which converted the gene list into a novel geneset library, compatible with the GSEAplots function. Finally, our custom geneset libraries (Old_MuSC_UP, Sarcopenia_UP and Sarcopenia_DOWN) were used to compute GSEA analysis on microgravity versus gravity samples.

### K. Proteomics Analysis

The conditioned media collected on day 7 of microgravity were analyzed by the Human Cytokine Array (GS4000, Raybiotech), which utilizes a matched pair of cytokine-specific antibodies for detection for each donor. The arrays were performed by RayBiotech per the manufacturer's instructions. The array consists of 200 cytokines, growth factors, proteases, soluble receptors and other proteins. The protein levels were normalized by DNA concentration and the DESEq2 package v1.30.1 was used for differential protein expression analysis. Statistical analysis was performed using the Wald test statistic, and the resulting p values were adjusted using the Benjaminin and Hochberg's approach for controlling the FDR<0.25 (n=2 donors per group).

### L. Protein-Protein Interaction Analysis

Protein-protein interaction (“PPI”) analysis of differentially expressed genes was based on the STRING database (https://string-db.org/). In brief, we retrieved a network for the list of proteins of interest using the multiple proteins search interface, specifying Homo sapiens in the Organisms field with default settings.

### M. Drug Screening in Microgravity

As proof of concept for using muscle-on-a-chip bioconstructs for drug screening, two drugs were assessed in microgravity conditions. The first was insulin-like growth factor-1 (IGF-1, 100 ng/ml, Peprotech), which is known to induce proliferation, growth, and regeneration (Alcazar et al., 2020; Ma et al., 2009; Machida and Booth, 2004; Yu et al., 2015). The second was SW033291 (200 nM, Cayman), a small molecule inhibitor of 15-hydroxyprostaglandin dehydrogenase (15-PGDH-i) that was previously shown to enhance myogenesis in previous studies (Ho et al., 2017). The drugs were diluted in induction media and loaded into syringes for media exchange on days 0 and 3 in separate bioreactors. On day 7, the conditioned media within the bioreactors were harvested at stored at −80° C. The muscle-on-a-chip bioconstructs within each chamber were treated with RNA Later stabilization agent at stored at −80° C. Upon sample return to Earth about two months later, the bioreactors were processed for RNA Sequencing and proteomics.

### N. Statistical Analysis

Statistical analysis of transcriptional and proteomics data is described above or in the supplemental experimental procedures. Where applicable, data is shown as mean±standard deviation. Statistical significance was accepted at P<0.05.

The following references of Example 1 are incorporated by reference herein in their respective entireties:
1. Alcazar, C. A., Hu, C., Rando, T. A., Huang, N. F., and Nakayama, K. H. (2020). Transplantation of insulin-like growth factor-1 laden scaffolds combined with exercise promotes neuroregeneration and angiogenesis in a preclinical muscle injury model. Biomater Sci 8, 5376-5389. 10.1039/d0bm00990c.
2. Andres-Mateos, E., Brinkmeier, H., Burks, T. N., Mejias, R., Files, D. C., Steinberger, M., Soleimani, A., Marx, R., Simmers, J. L., Lin, B., et al. (2013). Activation of serum/glucocorticoid-induced kinase 1 (SGK1) is important to maintain skeletal muscle homeostasis and prevent atrophy. EMBO Mol Med 5, 80-91. 10.1002/emmm.201201443.
3. Arthur, S. T., and Cooley, I. D. (2012). The effect of physiological stimuli on sarcopenia; impact of Notch and Wnt signaling on impaired aged skeletal muscle repair. Int J Biol Sci 8, 731-760. 10.7150/ijbs.4262.
4. Ascenzi, F., Barberi, L., Dobrowolny, G., Villa Nova Bacurau, A., Nicoletti, C., Rizzuto, E., Rosenthal, N., Scicchitano, B. M., and Musaro, A. (2019). Effects of IGF-1 isoforms on muscle growth and sarcopenia. Aging cell 18, e12954. 10.1111/acel.12954.
5. Baumgartner, R. N. (2000). Body composition in healthy aging. Ann. N. Y. Acad. Sci. 904, 437-448. 10.1111/j.1749-6632.2000.tb06498.x.
6. Baumgartner, R. N., Koehler, K. M., Gallagher, D., Romero, L., Heymsfield, S. B., Ross, R. R., Garry, P. J., and Lindeman, R. D. (1998). Epidemiology of sarcopenia among the elderly in New Mexico. Am. J. Epidemiol. 147, 755-763. 10.1093/oxfordjournals.aje.a009520.
7. Bjornson, C. R., Cheung, T. H., Liu, L., Tripathi, P. V., Steeper, K. M., and Rando, T. A. (2012). Notch signaling is necessary to maintain quiescence in adult muscle stem cells. Stem Cells 30, 232-242. 10.1002/stem.773.
8. Calve, S., Odelberg, S. J., and Simon, H. G. (2010). A transitional extracellular matrix instructs cell behavior during muscle regeneration. Dev. Biol. 344, 259-271. 10.1016/j.ydbio.2010.05.007.
9. Carlson, M. E., Hsu, M., and Conboy, I. M. (2008). Imbalance between pSmad3 and Notch induces CDK inhibitors in old muscle stem cells. Nature 454, 528-532. 10.1038/nature07034.
10. Choi, R. H., McConahay, A., Silvestre, J. G., Moriscot, A. S., Carson, J. A., and Koh, H. J. (2019). TRB3 regulates skeletal muscle mass in food deprivation-induced atrophy. FASEB J. 33, 5654-5666. 10.1096/fj.201802145RR.
11. Chung, L., and Ng, Y. C. (2006). Age-related alterations in expression of apoptosis regulatory proteins and heat shock proteins in rat skeletal muscle. Biochim. Biophys. Acta 1762, 103-109. 10.1016/j.bbadis.2005.08.003.
12. Conboy, I. M., Conboy, M. J., Smythe, G. M., and Rando, T. A. (2003). Notch-mediated restoration of regenerative potential to aged muscle. Science 302, 1575-1577. 10.1126/science.1087573.
13. Cruz-Jentoft, A. J., and Sayer, A. A. (2019). Sarcopenia. Lancet 393, 2636-2646. 10.1016/S0140-6736(19)31138-9.
14. da Silveira, W. A., Fazelinia, H., Rosenthal, S. B., Laiakis, E. C., Kim, M. S., Meydan, C., Kidane, Y., Rathi, K. S., Smith, S. M., Stear, B., et al. (2020). Comprehensive Multi-omics Analysis Reveals Mitochondrial Stress as a Central Biological Hub for Spaceflight Impact. Cell 183, 1185-1201 e1120. 10.1016/j.cell.2020.11.002.
15. Dehner, M., Hadjihannas, M., Weiske, J., Huber, O., and Behrens, J. (2008). Wnt signaling inhibits Forkhead box O3a-induced transcription and apoptosis through upregulation of serum- and glucocorticoid-inducible kinase 1. J. Biol. Chem. 283, 19201-19210. 10.1074/jbc.M710366200.
16. Fitts, R. H., Trappe, S. W., Costill, D. L., Gallagher, P. M., Creer, A. C., Colloton, P. A., Peters, J. R., Romatowski, J. G., Bain, J. L., and Riley, D. A. (2010). Prolonged space flight-induced alterations in the structure and function of human skeletal muscle fibres. The Journal of physiology 588, 3567-3592. 10.1113/jphysiol.2010.188508.
17. Fujimaki, S., Seko, D., Kitajima, Y., Yoshioka, K., Tsuchiya, Y., Masuda, S., and Ono, Y. (2018). Notch1 and Notch2 Coordinately Regulate Stem Cell Function in the Quiescent and Activated States of Muscle Satellite Cells. Stem Cells 36, 278-285. 10.1002/stem.2743.
18. Fujita, Y., Taniguchi, Y., Shinkai, S., Tanaka, M., and Ito, M. (2016). Secreted growth differentiation factor 15 as a potential biomarker for mitochondrial dysfunctions in aging and age-related disorders. Geriatr Gerontol Int 16 Suppl 1, 17-29. 10.1111/ggi.12724.
19. Furukawa, T., Tanimoto, K., Fukazawa, T., Imura, T., Kawahara, Y., and Yuge, L. (2018). Simulated microgravity attenuates myogenic differentiation via epigenetic regulations. NPJ Microgravity 4, 11. 10.1038/s41526-018-0045-0.
20. Gessler, L., Kurtek, C., Merholz, M., Jian, Y., and Hashemolhosseini, S. (2022). In Adult Skeletal Muscles, the Co-Receptors of Canonical Wnt Signaling, Lrp5 and Lrp6, Determine the Distribution and Size of Fiber Types, and Structure and Function of Neuromuscular Junctions. Cells 11. 10.3390/cells11243968.
21. Gilbert, W., Bragg, R., Elmansi, A. M., McGee-Lawrence, M. E., Isales, C. M., Hamrick, M. W., Hill, W. D., and Fulzele, S. (2019). Stromal cell-derived factor-1 (CXCL12) and its role in bone and muscle biology. Cytokine 123, 154783. 10.1016/j.cyto.2019.154783.
22. Ho, A. T. V., Palla, A. R., Blake, M. R., Yucel, N. D., Wang, Y. X., Magnusson, K. E. G., Holbrook, C. A., Kraft, P. E., Delp, S. L., and Blau, H. M. (2017). Prostaglandin E2 is essential for efficacious skeletal muscle stem-cell function, augmenting regeneration and strength. Proc. Natl. Acad. Sci. U.S.A 114, 6675-6684. 10.1073/pnas.1705420114.
23. Hu, C., Ayan, B., Chiang, G., Chan, A. H. P., Rando, T. A., and Huang, N. F. (2022). Comparative Effects of Basic Fibroblast Growth Factor Delivery or Voluntary Exercise on Muscle Regeneration after Volumetric Muscle Loss. Bioengineering (Basel) 9, 37. 10.3390/bioengineering9010037.
24. Huang, N. F., Okogbaa, J., Lee, J. C., Jha, A., Zaitseva, T. S., Paukshto, M. V., Sun, J. S., Punjya, N., Fuller, G. G., and Cooke, J. P. (2013). The modulation of endothelial cell morphology, function, and survival using anisotropic nanofibrillar collagen scaffolds. Biomaterials 34, 4038-4047. 10.1016/j.biomaterials.2013.02.036.
25. Huang, N. F., Patel, S., Thakar, R. G., Wu, J., Hsiao, B. S., Chu, B., Lee, R. J., and Li, S. (2006). Myotube assembly on nanofibrous and micropatterned polymers. Nano Lett 6, 537-542. 10.1021/n1060060o.
26. Huang, S., Wang, X., Yu, J., Tian, Y., Yang, C., Chen, Y., Chen, H., and Ge, H. (2020). LonP1 regulates mitochondrial network remodeling through the PINK1/Parkin pathway during myoblast differentiation. Am J Physiol Cell Physiol 319, C1020-C1028. 10.1152/ajpcell.00589.2019.
27. Ito, T., Nakanishi, Y., Yamaji, N., Murakami, S., and Schaffer, S. W. (2018). Induction of Growth Differentiation Factor 15 in Skeletal Muscle of Old Taurine Transporter Knockout Mouse. Biol. Pharm. Bull. 41, 435-439. 10.1248/bpb.b17-00969.
28. Juhl, O. J. t., Buettmann, E. G., Friedman, M. A., DeNapoli, R. C., Hoppock, G. A., and Donahue, H. J. (2021). Update on the effects of microgravity on the musculoskeletal system. NPJ Microgravity 7, 28. 10.1038/s41526-021-00158-4.
29. Kato, S., and Du, K. (2007). TRB3 modulates C2C12 differentiation by interfering with Akt activation. Biochem. Biophys. Res. Commun. 353, 933-938. 10.1016/j.bbrc.2006.12.161.
30. Kawase, T., Ichikawa, H., Ohta, T., Nozaki, N., Tashiro, F., Ohki, R., and Taya, Y. (2008). p53 target gene AEN is a nuclear exonuclease required for p53-dependent apoptosis. Oncogene 27, 3797-3810. 10.1038/onc.2008.32.
31. Kawase, T., Ohki, R., Shibata, T., Tsutsumi, S., Kamimura, N., Inazawa, J., Ohta, T., Ichikawa, H., Aburatani, H., Tashiro, F., and Taya, Y. (2009). PH domain-only protein PHLDA3 is a p53-regulated repressor of Akt. Cell 136, 535-550. 10.1016/j.cell.2008.12.002.
32. Kim, H., Kim, K. M., Kang, M. J., and Lim, S. (2020). Growth differentiation factor-15 as a biomarker for sarcopenia in aging humans and mice. Exp. Gerontol. 142, 111115. 10.1016/j.exger.2020.111115.
33. Kim-Muller, J. Y., Song, L., LaCarubba Paulhus, B., Pashos, E., Li, X., Rinaldi, A., Joaquim, S., Stansfield, J. C., Zhang, J., Robertson, A., et al. (2023). GDF15 neutralization restores muscle function and physical performance in a mouse model of cancer cachexia. Cell reports 42, 111947. 10.1016/j.celrep.2022.111947.
34. Kyle, U. G., Genton, L., Hans, D., Karsegard, V. L., Michel, J. P., Slosman, D. O., and Pichard, C. (2001). Total body mass, fat mass, fat-free mass, and skeletal muscle in older people: cross-sectional differences in 60-year-old persons. J. Am. Geriatr. Soc. 49, 1633-1640. 10.1046/j.1532-5415.2001.t01-1-49272.x.
35. Leeuwenburgh, C. (2003). Role of apoptosis in sarcopenia. J. Gerontol. A. Biol. Sci. Med. Sci. 58, 999-1001. 10.1093/gerona/58.11.m999.
36. Liu, L., Charville, G. W., Cheung, T. H., Yoo, B., Santos, P. J., Schroeder, M., and Rando, T. A. (2018). Impaired Notch Signaling Leads to a Decrease in p53 Activity and Mitotic Catastrophe in Aged Muscle Stem Cells. Cell Stem Cell 23, 544-556 e544. 10.1016/j.stem.2018.08.019.
37. Luo, D., Renault, V. M., and Rando, T. A. (2005). The regulation of Notch signaling in muscle stem cell activation and postnatal myogenesis. Semin. Cell Dev. Biol. 16, 612-622. 10.1016/j.semcdb.2005.07.002.
38. Ma, Q. L., Yang, T. L., Yin, J. Y., Peng, Z. Y., Yu, M., Liu, Z. Q., and Chen, F. P. (2009). Role of insulin-like growth factor-1 (IGF-1) in regulating cell cycle progression. Biochem. Biophys. Res. Commun. 389, 150-155. 10.1016/j.bbrc.2009.08.114.
39. Machida, S., and Booth, F. W. (2004). Insulin-like growth factor 1 and muscle growth: implication for satellite cell proliferation. Proc. Nutr. Soc. 63, 337-340. 10.1079/PNS2004354.
40. Mauro, A. (1961). Satellite cell of skeletal muscle fibers. The Journal of biophysical and biochemical cytology 9, 493-495. 10.1083/jcb.9.2.493.
41. Morgan, J. E., and Partridge, T. A. (2003). Muscle satellite cells. Int. J. Biochem. Cell Biol. 35, 1151-1156. 10.1016/s1357-2725(03)00042-6.
42. Nakayama, K. H., Alcazar, C., Yang, G., Quarta, M., Paine, P., Doan, L., Davies, A., Rando, T. A., and Huang, N. F. (2018). Rehabilitative exercise and spatially patterned nanofibrillar scaffolds enhance vascularization and innervation following volumetric muscle loss. Npj Regen Med 3, 16. 10.1038/s41536-018-0054-3.
43. Nakayama, K. H., Quarta, M., Paine, P., Alcazar, C., Karakikes, I., Garcia, V., Abilez, O. J., Calvo, N. S., Simmons, C. S., Rando, T. A., and Huang, N. F. (2019). Treatment of volumetric muscle loss in mice using nanofibrillar scaffolds enhances vascular organization and integration. Communications biology 2, 170. 10.1038/s42003-019-0416-4.
44. Nakayama, K. H., Surya, V. N., Gole, M., Walker, T. W., Yang, W., Lai, E. S., Ostrowski, M. A., Fuller, G. G., Dunn, A. R., and Huang, N. F. (2016). Nanoscale Patterning of Extracellular Matrix Alters Endothelial Function under Shear Stress. Nano Lett 16, 410-419. 10.1021/acs.nanolett.5b04028.
45. Nikawa, T., Ishidoh, K., Hirasaka, K., Ishihara, I., Ikemoto, M., Kano, M., Kominami, E., Nonaka, I., Ogawa, T., Adams, G. R., et al. (2004). Skeletal muscle gene expression in space-flown rats. FASEB J. 18, 522-524. 10.1096/fj.03-0419fje.
46. Palla, A. R., Ravichandran, M., Wang, Y. X., Alexandrova, L., Yang, A. V., Kraft, P., Holbrook, C. A., Schurch, C. M., Ho, A. T. V., and Blau, H. M. (2021). Inhibition of prostaglandin-degrading enzyme 15-PGDH rejuvenates aged muscle mass and strength. Science 371, abc8059. 10.1126/science.abc8059.
47. Parafati, M., Giza, S., Shenoy, T. S., Mojica-Santiago, J. A., Hopf, M., Malany, L. K., Platt, D., Moore, I., Jacobs, Z. A., Kuehl, P., et al. (2023). Human skeletal muscle tissue chip autonomous payload reveals changes in fiber type and metabolic gene expression due to spaceflight. NPJ Microgravity 9, 77. 10.1038/s41526-023-00322-y.
48. Pedruzzi, E., Guichard, C., Ollivier, V., Driss, F., Fay, M., Prunet, C., Marie, J. C., Pouzet, C., Samadi, M., Elbim, C., et al. (2004). NAD(P)H oxidase Nox-4 mediates 7-ketocholesterol-induced endoplasmic reticulum stress and apoptosis in human aortic smooth muscle cells. Mol. Cell. Biol. 24, 10703-10717. 10.1128/MCB.24.24.10703-10717.2004.
49. Perez, K., Ciotlos, S., McGirr, J., Limbad, C., Doi, R., Nederveen, J. P., Nilsson, M. I., Winer, D. A., Evans, W., Tarnopolsky, M., et al. (2022). Single nuclei profiling identifies cell specific markers of skeletal muscle aging, frailty, and senescence. Aging (Albany NY) 14, 9393-9422. 10.18632/aging.204435.
50. Puchert, M., Adams, V., Linke, A., and Engele, J. (2016). Evidence for the involvement of the CXCL12 system in the adaptation of skeletal muscles to physical exercise. Cell. Signal. 28, 1205-1215. 10.1016/j.cellsig.2016.05.019.
51. Radugina, E. A., Almeida, E. A. C., Blaber, E., Poplinskaya, V. A., Markitantova, Y. V., and Grigoryan, E. N. (2018). Exposure to microgravity for 30 days onboard Bion M1 caused muscle atrophy and impaired regeneration in murine femoral Quadriceps. Life Sci Space Res (Amst) 16, 18-25. 10.1016/j.lssr.2017.08.005.
52. Reid, K. F., and Fielding, R. A. (2012). Skeletal muscle power: a critical determinant of physical functioning in older adults. Exerc. Sport Sci. Rev. 40, 4-12. 10.1097/JES.0b013e31823b5f13.
53. Ren, Q., Chen, J., and Liu, Y. (2021). LRP5 and LRP6 in Wnt Signaling: Similarity and Divergence. Front Cell Dev Biol 9, 670960. 10.3389/fcell.2021.670960.
54. Ren, Z., Ahn, E. H., Do, M., Mair, D. B., Monemianesfahani, A., Lee, P. H. U., and Kim, D. H. (2024). Simulated microgravity attenuates myogenesis and contractile function of 3D engineered skeletal muscle tissues. NPJ Microgravity 10, 18. 10.1038/s41526-024-00353-z.
55. Saleem, S., and Biswas, S. C. (2017). Tribbles Pseudokinase 3 Induces Both Apoptosis and Autophagy in Amyloid-beta-induced Neuronal Death. J. Biol. Chem. 292, 2571-2585. 10.1074/jbc.M116.744730.
56. Scholz, F., Schulte, A., Adamski, F., Hundhausen, C., Mittag, J., Schwarz, A., Kruse, M. L., Proksch, E., and Ludwig, A. (2007). Constitutive expression and regulated release of the transmembrane chemokine CXCL16 in human and murine skin. J. Invest. Dermatol. 127, 1444-1455. 10.1038/sj.jid.5700751.
57. Shang, G. K., Han, L., Wang, Z. H., Liu, Y. P., Yan, S. B., Sai, W. W., Wang, D., Li, Y. H., Zhang, W., and Zhong, M. (2020). Sarcopenia is attenuated by TRB3 knockout in aging mice via the alleviation of atrophy and fibrosis of skeletal muscles. J Cachexia Sarcopenia Muscle 11, 1104-1120. 10.1002/jcsm.12560.
58. Siu, P. M., Pistilli, E. E., and Alway, S. E. (2005). Apoptotic responses to hindlimb suspension in gastrocnemius muscles from young adult and aged rats. American journal of physiology. Regulatory, integrative and comparative physiology 289, R1015-1026. 10.1152/ajpregu.00198.2005.
59. Takahashi, H., Nakamura, A., and Shimizu, T. (2021). Simulated microgravity accelerates aging of human skeletal muscle myoblasts at the single cell level. Biochem. Biophys. Res. Commun. 578, 115-121. 10.1016/j.bbrc.2021.09.037.
60. Trappe, T. (2009). Influence of aging and long-term unloading on the structure and function of human skeletal muscle. Applied physiology, nutrition, and metabolism=Physiologie appliquee, nutrition et metabolisme 34, 459-464. 10.1139/H09-041.
61. van Velthoven, C. T. J., and Rando, T. A. (2019). Stem Cell Quiescence: Dynamism, Restraint, and Cellular Idling. Cell Stem Cell 24, 213-225. 10.1016/j.stem.2019.01.001.
62. Vandenburgh, H., Chromiak, J., Shansky, J., Del Tatto, M., and Lemaire, J. (1999). Space travel directly induces skeletal muscle atrophy. FASEB J. 13, 1031-1038. 10.1096/fasebj.13.9.1031.
63. Vitry, G., Finch, R., McStay, G., Behesti, A., Dejean, S., Larose, T., Wotring, V., and da Silveira, W. A. (2022). Muscle atrophy phenotype gene expression during spaceflight is linked to a metabolic crosstalk in both the liver and the muscle in mice. iScience 25, 105213. 10.1016/j.isci.2022.105213.
64. Yakovlev, A. G., Di Giovanni, S., Wang, G., Liu, W., Stoica, B., and Faden, A. I. (2004). BOK and NOXA are essential mediators of p53-dependent apoptosis. J. Biol. Chem. 279, 28367-28374. 10.1074/jbc.M313526200.
65. Yamada, T., Miyabe, Y., Ueki, S., Fujieda, S., Tokunaga, T., Sakashita, M., Kato, Y., Ninomiya, T., Kawasaki, Y., Suzuki, S., and Saito, H. (2019). Eotaxin-3 as a Plasma Biomarker for Mucosal Eosinophil Infiltration in Chronic Rhinosinusitis. Front Immunol 10, 74. 10.3389/fimmu.2019.00074.
66. Yartseva, V., Goldstein, L. D., Rodman, J., Kates, L., Chen, M. Z., Chen, Y. J., Foreman, O., Siebel, C. W., Modrusan, Z., Peterson, A. S., and Jovicic, A. (2020). Heterogeneity of Satellite Cells Implicates DELTA1/NOTCH2 Signaling in Self-Renewal. Cell reports 30, 1491-1503 e1496. 10.1016/j.celrep.2019.12.100.
67. Youm, T. H., Woo, S. H., Kwon, E. S., and Park, S. S. (2019). NADPH Oxidase 4 Contributes to Myoblast Fusion and Skeletal Muscle Regeneration. Oxid Med Cell Longev 2019, 3585390. 10.1155/2019/3585390.
68. Yu, M., Wang, H., Xu, Y., Yu, D., Li, D., Liu, X., and Du, W. (2015). Insulin-like growth factor-1 (IGF-1) promotes myoblast proliferation and skeletal muscle growth of embryonic chickens via the PI3K/Akt signalling pathway. Cell Biol. Int. 39, 910-922. 10.1002/cbin.10466.
69. Zhang, W., Sun, W., Gu, X., Miao, C., Feng, L., Shen, Q., Liu, X., and Zhang, X. (2022). GDF-15 in tumor-derived exosomes promotes muscle atrophy via Bcl-2/caspase-3 pathway. Cell Death Discov 8, 162. 10.1038/s41420-022-00972-z.

### Example 2

### Simulated Microgravity Platform

### A. Cell/Tissue Adaptation to Simulated Microgravity

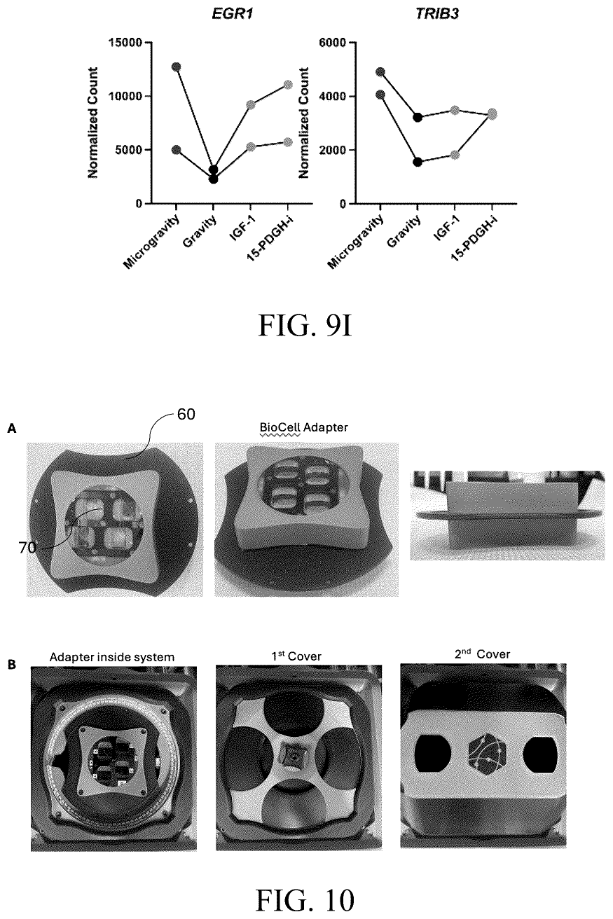

Simulated microgravity can be performed on muscle cells within conventional tissue culture dishes or tissue engineered muscle tissues. Conventional cell culture dishes can be adapted to simulated microgravity by filling the wells completely in media, followed by the application of a breathable adhesive tape to prevent leakage of media. For engineering of muscle tissue constructs, we employed nano-scale spatial guidance cues from aligned nanofibrillar collagen scaffolds that can guide muscle progenitor/myoblast fusion into organized myotubes, creating a biomimetic aligned skeletal muscle bioconstruct. The bioreactors or dishes are then placed into simulated microgravity equipment, such as the Cose clinostat (FIG. 10), and secured in place with the relevant adaptors and covers.

B. Determining Key Drugs Compounds that Reverse Sarcopenia-Like Characteristics of Cultured Human Myoblasts in 96 Well Dishes in a 2D Simulated Microgravity Environment

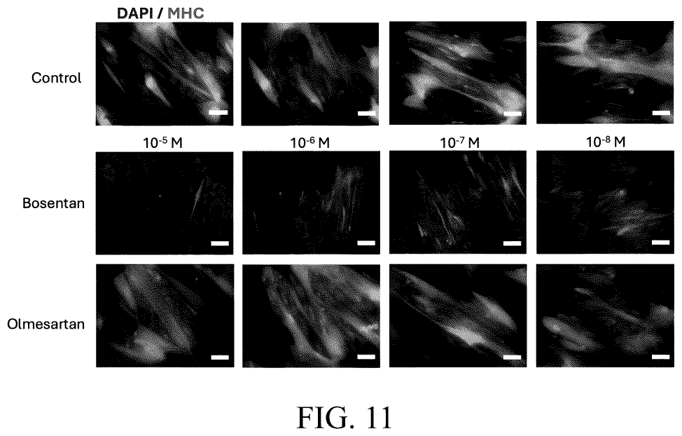

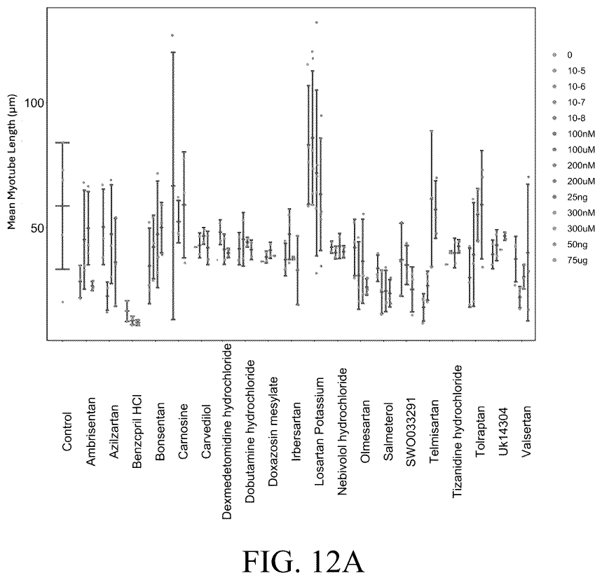

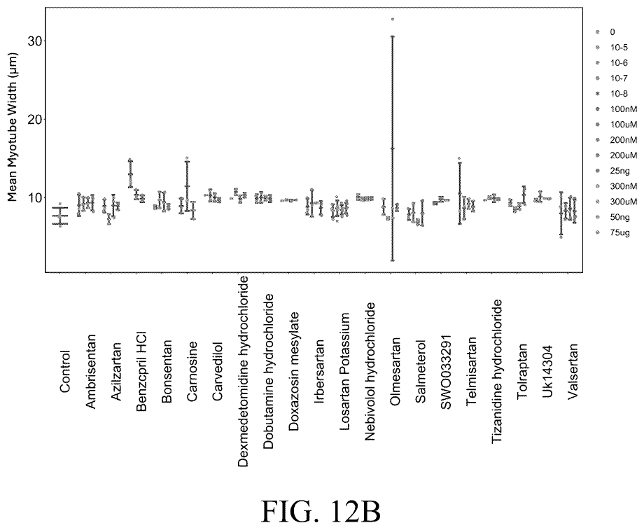

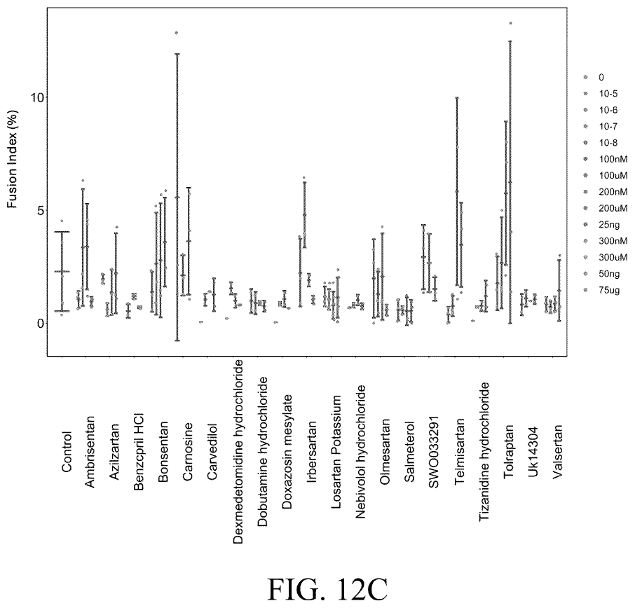

We have performed drug screening studies of in vitro cultured human myoblasts cells in a 96 well plate, where up to 8 samples can be studied at 4 different concentrations (10⁻⁵ to 10⁻⁸M) in triplicate in simulated microgravity (10⁻³ g) conditions. As we are interested in determining the effects of these compounds on skeletal muscle regeneration, any positive characteristics that are seen to increase myotube formation must be noted. To ensure myotube presence and differences are noted the samples, consisting of myoblasts in differentiation media (DMEM+2% Horse Serum), treated with one of the compounds at varying concentrations, can be stained for Myosin Heavy Chain and then fluorescent images can be acquired. Representative images of drugs showing varying effect when compared to the control can be found in FIG. 11. Using this platform, dozens of drugs were screened and analyzed using three different metrics: mean myotube length, mean myotube width, and a fusion index, which describes the percentage of nuclei that can be found inside of a myotube in relation to the overall number of nuclei in an image. Any drug found showing >10% increase than those found in the control group were selected to be subjected to n=3 studies in order to validate the results. Shown in FIGS. 12A, 12B, and 12C are representative drugs that showed some benefit to either metrics of myotube length, width, or myotube incorporation, respectively.

### Example 3

### Specific Aims

According to the American Community Survey (2015-2019) from the US Census Bureau, there are more than 18 million Veterans over the age of 65 years. This aging population accounts for nearly 50% of the total Veteran population, but only 18% of non-Veterans. This disproportionately large population of aging Veterans is susceptible to ailments, leading to hospitalization-induced unloading/disuse muscle atrophy. When older patients undergo hospitalization, there is an approximate 30-55% decline in ambulatory function and 65% decline in daily activities [1-3]. Moreover, this decline in muscle conditioning during hospitalization leads to increased susceptibility to readmission after discharge [4]. Importantly, there are no FDA-approved drugs to treat atrophy. Therefore, identifying interventions to treat disuse atrophy is a major public health challenge.

The development of interventions hinges on the ability to model or mimic disuse atrophy. Existing muscle disuse models include bed rest, dry immersion, or unilateral limb suspension induce muscle loss. However, such models rely primarily on in vivo subjects, which severely limit the ability to perform higher-throughput drug testing. In recent decades, the use of microgravity as a unique stimulus of muscle atrophy has gained traction. Assessments of structural and functional muscle changes during prolonged spaceflights or simulated microgravity have contributed to our knowledge of disuse atrophy [5, 6]. Under conditions of spaceflight, muscle cells undergo accelerated senescence [7] and muscle mass is reduced dramatically over four weeks [8]. Additionally, a larger body of work derived from in vitro cultured myoblasts in microgravity or simulated microgravity further support the finding that microgravity impairs myogenesis [7, 9], which is a contributing factor to muscle atrophy [10]. However, the opportunity to perform studies in real microgravity conditions is limited, and so it is more feasible to utilize commercial equipment to simulate microgravity (10⁻³ g) conditions instead.

Accordingly, the objective is to develop and validate a novel in vitro model of disuse atrophy using engineered skeletal muscle under simulated microgravity conditions, and to apply this platform for drug screening. Simulated microgravity can be conveniently mimicked by commercial equipment such as a clinostat or rotating wall vessel. This proposal is based on our laboratory's expertise in engineering artificial skeletal muscle-on-a-chip bioconstructs that exhibit muscle contractility and myofiber formation capacity [11]. Validation of the disclosed in vitro model of engineered muscle in simulated microgravity is essential for its acceptance by the scientific community, and it can decrease the reliance on in vivo models.

### A. Specific Aim 1. To Quantitatively Assess the Efficacy of In Vitro Engineered Skeletal Muscle in Simulated Microgravity to Recapitulate Salient Features of Disuse Muscle Atrophy.

Engineered human skeletal muscle can be cultured in simulated microgravity conditions within a three-dimensional clinostat or under normal gravity conditions. To quantify the kinetics of microgravity on muscle atrophy, the muscle-on-a-chip bioconstructs can be evaluated for muscle contractility, myotube formation and organization, and transcriptional profiling for up to 14 days, and then compared to corresponding samples in normal gravity. Additionally, the transcriptional data can be compared to RNASeq datasets derived from clinical muscle unloading studies, in order to validate this in vitro model of muscle atrophy.

### B. Specific Aim 2. To Apply the Engineered Skeletal Muscle in Simulated Microgravity Platform for Pilot Testing of Drugs for Treatment of Disuse Muscle Atrophy.

Engineered skeletal muscle in simulated microgravity can be screened with a collection of FDA-approved repurposed drugs for up to 14 days. Initial studies can be performed to identify a range in concentration that is non-cytotoxic. In the setting of simulated microgravity, the drugs can be applied to the engineered muscles at the start of induction media (day 0) and maintained for up to 14 days. At each time point, muscle contractility can be performed, along with immunofluorescence staining of myosin heavy chain for quantification of myotube formation. Drugs showing >30% improvement in these output measures in simulated microgravity can be further examined by RNA sequencing to reveal the mechanism by which the drugs act. A reference compound can also be included, namely SW033291 that has shown benefit in our preliminary in vitro studies in microgravity, as well as in preclinical models of muscle atrophy [12].

### C. Significance

The findings from this study are expected to increase the use of clinostat-based simulated microgravity to study muscle unloading. Our disclosed in vitro system can be readily adopted for research use, since cost-effective simulated microgravity clinostats are now commercially available. In addition, tissue engineered skeletal muscle can be reproducibly prepared. These recent advancements can expand the availability of this in vitro platform for broader research use and decrease the reliance on animal models. Such a platform with drug screening capability can greatly improve and accelerate the pace of identifying drugs to study and treat muscle disuse atrophy in Veterans.

### Background and Significance

### A. Unloading/Disuse Muscle Atrophy

According to the American Community Survey (2015-2019) from the US Census Bureau, there are more than 18 million Veterans over the age of 65 years. This aging population accounts for nearly 50% of the total Veteran population, but only 18% of non-Veterans. This disproportionately large population of aging Veterans is susceptible to ailments, leading to unloading/disuse muscle atrophy associated with sustained bed rest, hospitalization, or limb immobilization. Muscle atrophy is characterized by the reduction of muscle mass and a decline in muscle function, as well as an impairment in myogenesis [10]. Even among healthy young individuals, immobilization of the leg resulted in a 17% reduction in muscle mass over 8 weeks [13]. In another study, healthy women who underwent bedrest for 59 days experienced 20-30% decline in muscle volume of the calf and thigh muscles, along with impaired muscle function [14]. Compared to younger individuals, older individuals have a heightened degree of atrophy, in which 10 days of bed rest resulted in 11-12% functional decline per week, based on knee extensor peak torque [15]. When elderly patients undergo hospitalization, there is an approximate 30-55% decline in ambulatory function and 65% decline in daily activities [1-3]. Moreover, this decline in muscle conditioning during hospitalization leads to increased susceptibility to readmission after discharge [4]. To combat unloading/disuse muscle atrophy, existing recommendations of dietary modifications and supplement intake have shown inconsistent benefit [13, 14, 16]. Countermeasures such as exercise [14] have been shown to be effective in improving muscle function and mass, but the levels could not be normalized to pre-immobilization levels. Despite the possible benefit of exercise in reversing muscle atrophy, not all patients are medically able to exercise after the period of unloading/disuse. Importantly, there are currently no FDA-approved drugs to treat unloading/disuse atrophy. Therefore, identifying interventions to treat disuse unloading/disuse muscle atrophy is a major public health challenge for Veterans and civilians.

### B. Microgravity As a Novel Unloading Model

Developing interventions hinges on the ability to model or mimic disuse atrophy. Existing muscle unloading/disuse models involve bed rest, dry immersion, or unilateral limb suspension induce muscle loss. However, such models rely primarily on in vivo subjects, which severely limit the ability to perform higher-throughput drug testing. In recent decades, the use of real or simulated microgravity (10⁻³ g) as a unique stimulus of muscle atrophy has gained traction, thereby opening the possibility for in vitro models of muscle unloading. Assessments of structural and functional muscle changes during prolonged spaceflights or simulated microgravity have contributed to our knowledge of disuse atrophy [5, 6]. Under conditions of spaceflight, muscle cells undergo accelerated senescence [7] and muscle mass is reduced dramatically over four weeks [8]. Single fiber proteomics have been previously performed to compare bed rest induced muscle atrophy and microgravity conditions [17]. The results from this study show that the molecular remodeling resulting from bed rest induced atrophy shared similar features to that of muscle samples derived from astronauts after a half-year mission in space. Additionally, a larger body of work derived from in vitro cultured muscle progenitor cells (MPCs) in microgravity or simulated microgravity further support the finding that microgravity impairs myogenesis [7, 9] as a contributing factor to muscle atrophy [10].

### C. Simulated Microgravity Equipment is Economically Feasible for Improved Accessibility

However, since the opportunity to perform studies in real microgravity conditions is limited, it is more feasible to utilize commercial equipment to simulate microgravity (10⁻³ g) conditions instead. Commercial equipment that can simulate microgravity (10⁻³ g) conditions include the clinostat, random positioning machine (RPM), and rotating wall vessel. Clinostats operate by rotating a sample about one axis (two-dimensional (2D) clinostat) or two axes (three-dimensional (3D) clinostat) such that gravity does not act on any one direction and the gravitational vector of the sample approaches zero [16]. Using a similar principle, RPMs act as 3D clinostats that rotate about two axes. Rotating wall vessels rotate around a horizontal axis in which the gravitational forces are normalized by hydrodynamic drag forces, leading to microgravity-like conditions [18]. Since rotating wall vessels are more commonly used in conjunction with suspension cells or microcarrier beads, 3D clinostats are better suited for adherent cells and engineered muscle tissues.

In the advent of technological advancements, the cost of 3D clinostats recently became more economically feasible for improved accessibility to this technology. Traditionally, commercial 3D clinostats such as the Airbus RPM 2.0 (Yuri, Germany) and GRAVITE (Space Bio Laboratories, Japan) cost over $50,000 USD [19, 20]. Recently, a cost-effective 3D clinostat became commercially available (SciSpinner MAX 3D Clinostat, CoSE) and is based within the US. The SciSpinner is a 3D clinostat that is much more economically feasible at $5,000, owing to 3D printed parts that are economical to manufacture, yet are durable [21]. Other advantages of the SciSpinner include that it can: 1) fit into standard cell culture incubators; and 2) sample holders can be custom 3D-printed to fit the specific dimensions of bioreactors that house the samples. Importantly, the SciSpinner has been validating against commercial clinostats with biological samples and have shown to have similar microgravity effects [21]. We have already available in our laboratory the SciSpinner 3D clinostat and have found it easy to assemble and operate.

### D. Engineered Muscle in Microgravity Platform for Modeling Muscle Atrophy

This proposal originates from exciting preliminary data in which we developed biomimetic engineered skeletal muscle-on-a-chip platform that mimics the physiological parallel-aligned orientation of native muscle fibers [22]. Our novel approach employs parallel-aligned nanofibrillar scaffolds that guide muscle progenitor cell (“MPC”) fusion into parallel-aligned skeletal muscle myotubes, forming a biomimetic muscle with physiologic spatial patterning. We have previously applied this tissue engineering platform for therapeutic applications of treatment traumatic muscle injury [11]. We believe this platform may be useful for drug testing applications in simulated microgravity. Moreover, as part of a project funded by the National Science Foundation and the Center for the Advancement of Science in Space, we modeled muscle atrophy aboard the International Space Station National Laboratories using tissue engineered skeletal muscle. It is contemplated that this established engineered muscle-on-a-chip platform can be applied to study the effects of simulated microgravity on muscle atrophy.

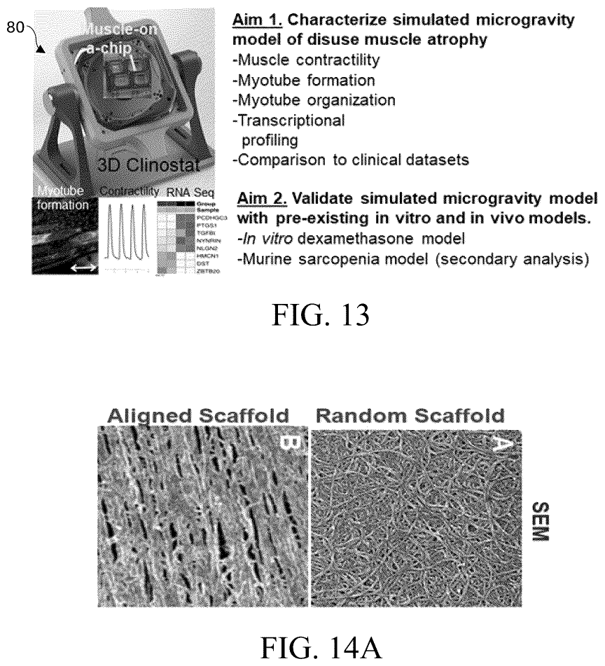

Accordingly, the objective is to develop and validate a novel in vitro model of disuse atrophy using engineered skeletal muscle under simulated microgravity conditions, and to apply this platform for pilot drug screening (FIG. 13). This proposal is based on our laboratory's expertise in engineering artificial skeletal muscle that exhibits muscle contractility and myofiber formation capacity, along with our already established expertise in adapting an engineered muscle-on-a-chip platform to model microgravity.

### Innovation

Multiple aspects of this example and the current disclosure are innovative, including but not limited to the following:

### A. Simulated Microgravity as a Novel Model of Muscle Atrophy.

While being more accessible than real microgravity, simulated microgravity has the potential to mimic salient aspects of muscle atrophy, which is intended to serve as an alternative to preclinical or clinical models.

### B. Engineered Skeletal Muscle as a Platform to Study Muscle Atrophy.

This project is based on our expertise in tissue engineering of skeletal muscle, we are first to develop a muscle-on-a-chip platform that is compatible with simulated microgravity stimulation.

### C. Testing of FDA-Approved Drugs for Near-Term Clinical Translation.

FDA-approved drugs have already passed safety testing, so therefore these drugs may have near-term clinical translation potential.

### D. Exemplary Usage of SW033291.

This small molecular inhibitor of prostaglandin E2 (PGE2)-degrading enzyme (15-PGDH) as shown to be safe and effective in treating aging-associated muscle atrophy [12], but has yet to be tested in treating unloading/disuse atrophy. This drug can be included as a reference drug for comparison to a library of FDA-approved drugs.

### E. 'Omics Analysis of how the Drugs Modulate Salient Qualities of Unloading/Disuse Atrophy.

This proposal integrates multidisciplinary approaches and concepts by synergizing drug screening and tissue engineering platforms with MPC biology and big data omics approaches.

### Preliminary Data

This proposal arises from exciting preliminary data in which we developed an effective tissue engineering platform that can support skeletal muscle survival, function, and regeneration in vivo by mimicking physiological muscle myofiber structure. Our novel approach can employ nano-scale spatial guidance cues from aligned nanofibrillar collagen scaffolds that guide MPC differentiation into organized myotubes, creating a biomimetic aligned skeletal muscle bioconstruct. These engineered scaffolds were successfully launched to the ISSNL previously, in which we showed that the engineered muscle in microgravity show evidence of increased muscle wasting, compared to in normal gravity conditions. These preliminary data are described below:

### A. Fabrication of Aligned Nanofibrillar Scaffolds for Engineering Aligned Skeletal Muscle.

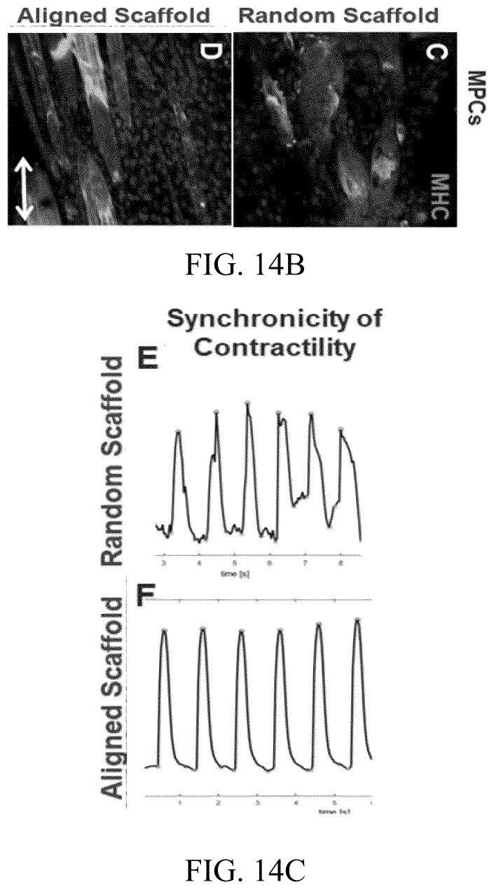

The scaffolds were produced by a facile method of extruding bovine monomeric collagen type I from a fine needle into a pH 7.4 buffer, forming collagen strips (500 μm thick) along the direction of extrusion [23-26]. Importantly, extrusion of collagen at high speed induced the formation of parallel-oriented nanofibrils with nanofibril diameters of 30-50 nm, based on scanning electron microscopy (FIG. 14A) [11]. By reducing the speed of extrusion, the fibrillogenesis became more disorganized, forming randomly oriented nanofibrillar scaffolds (FIG. 14A).

### B. In Vitro Characterization of Engineered Skeletal Muscle Organization and Contractility.

Murine MPCs were seeded into the scaffolds in media that supported the formation of multi-nucleated myotubes. In the aligned scaffolds, myotubes prefer to form along the direction of the nanofibrils and contract synchronously in response to electrical stimulation (1 Hz), whereas the myotubes were highly disorganized on the randomly oriented scaffold and were less synchronized in contractility, as assessed by visualization of myosin heavy chain (MyHC) and quantitative contractility analysis (FIGS. 14B, 14C) [11]. Together, this data demonstrated that our engineered muscle can possess physiologically relevant organization and function, which makes it a good in vitro model for drug screening.

### C. Housing of Muscles-on-a Chip in Microgravity in Bioreactors.

In prior space research, we developed bioreactors (termed “BioCells”) suitable for drug screening of engineered muscle (FIGS. 3A-3D) in microgravity. Each well can contain clamps for securing glass chips, onto which the muscle-on-a-chip can be attached (FIG. 3A). The muscle-on-a-chip bioconstructs can receive nutrient and oxygen exchange through independent inlet and outlet ports using conventional syringes (FIG. 3D). BioCells can fit within housing units that allow for gas exchange and 37° C. temperature maintenance during launch and on orbit in the incubator aboard the ISSNL (FIG. 3C).

### D. Engineered Muscle-On-A-Chip Platform in Real Microgravity Provides Proof-of-Concept for Simulated Microgravity Platform.

In our previously funded NSF/CASIS award, we successfully launched our engineered muscles-on-a-chip to the ISSNL, in which the space astronauts performed experiments to study muscle myotube formation in microgravity (FIGS. 3E, 3F), while on Earth we performed parallel gravity experiments.

E. Transcriptional and Morphological Changes in Microgravity that Mimics Aspects of Muscle Atrophy.

The engineered muscle-on-a-chip platform was exposed to microgravity aboard the ISSNL for 7 days, followed by bulk RNA sequencing analysis. In preliminary data showing engineered muscle response to microgravity from two independent primary human MPC donors, we showed that the top 20 genes that are significantly downregulated in microgravity (FIG. 5G). In particular, NOTCH2, which is an important marker of muscle satellite cell self-renewal and stem cell maintenance fate decision, was significantly downregulated in flight, suggesting an impairment in muscle satellite cells that is consistent with atrophy [27]. Another feature of muscle atrophy is impaired extracellular matrix (“ECM”) remodeling. Among the ECM components was significant downregulation of COL12A1, LAMA2, and HSPG2, whose downregulation is associated with muscle weakness [28] and skeletal development defects [29].

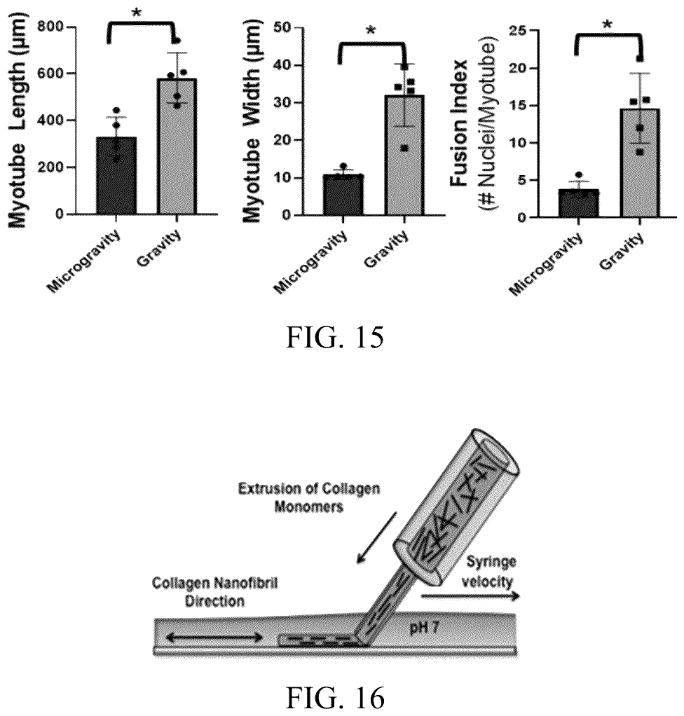

This transcriptomic data was further supported by quantification of in vitro myotube length, based on immunofluorescence staining of skeletal myosin heavy chain. Myosin heavy chain expression that stains for myotubes were compared in the donor muscles exposed to microgravity or gravity. These findings show that the myotubes formed in microgravity were significantly shorter and thinner, compared to gravity conditions (FIG. 15). This finding is in agreement with published results of in vitro cultured myoblasts in which microgravity was shown to impair muscle formation that is associated with muscle atrophy [7, 9]. Together, this data suggests that microgravity conditions induce a cellular phenotype that is associated with characteristics of muscle atrophy, including impaired ECM remodeling and impaired myogenesis.

### F. Preclinical Testing Identifies SW033291 Having Therapeutic Benefit for Treatment of Aging-Related Muscle Atrophy.

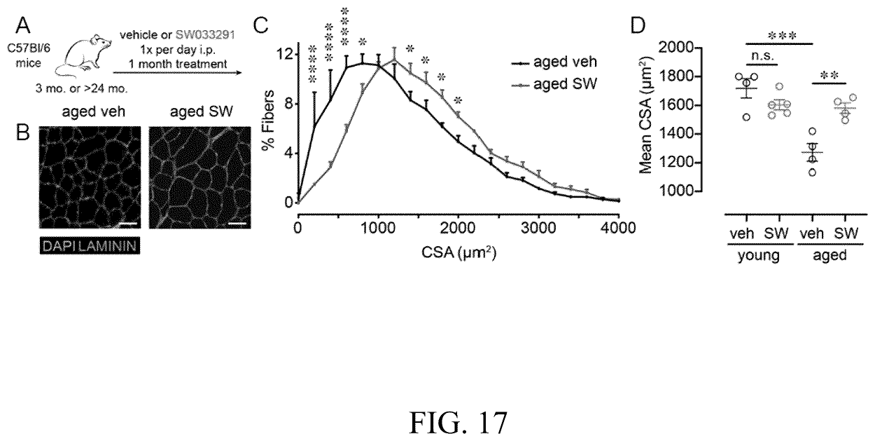

Muscle-derived prostaglandin E2 (PGE2), a fatty acid derivative of arachidonic acid, is an important inducer of myogenesis [30] by activating the expansion of satellite cells and MPCs that precedes muscle formation [30, 31]. However, owing to PGE2's rapid degradation in vivo, one strategy to increase the abundance of PGE2 is to block a negative regulator of PGE2 known as prostaglandin E2 (PGE2)-degrading enzyme (15-PGDH). Palla et al. that showed that daily injection of SW033291 could improve muscle mass and strength in aged mice in response to aging-related muscle atrophy known as sarcopenia (FIG. 17) [12]. Additionally, histological analysis demonstrated that the cross-sectional area in aged mice treated with drug showed a shift in increasing mean cross-sectional area (FIG. 17), along with improved muscle force generation.

### G. Space Launch Dataset Demonstrates Efficacy of SW033291 to Partially Prevent Muscle Atrophy.

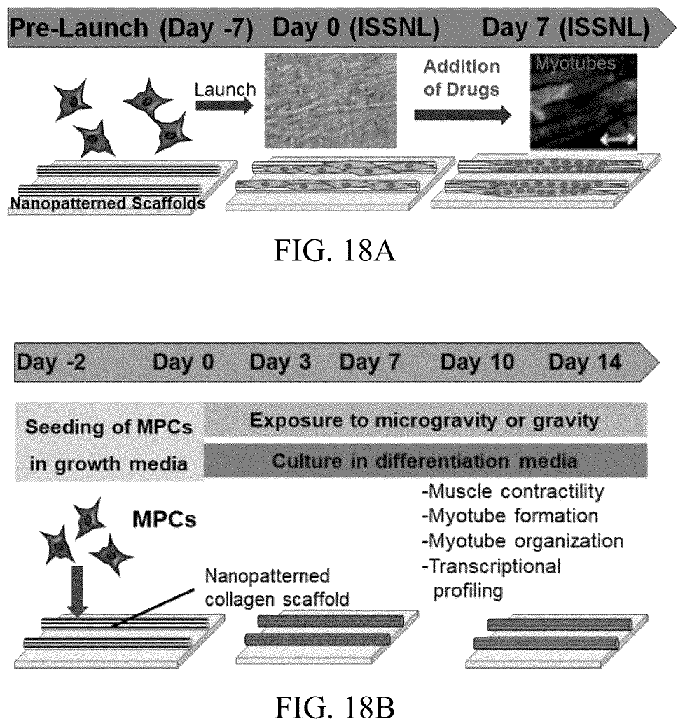

Having established the efficacy of the microgravity platform to mimic aspects of muscle atrophy, we performed a proof-of-concept drug screen to identify putative targets that can prevent muscle atrophy in microgravity conditions. In these studies, our muscle-on-a-chip platform was treated with drugs for 7 days in microgravity before RNA Sequencing analysis. With the knowledge of SW033291 reportedly showing therapeutic benefit in mice with aged-related atrophy, we exposed the muscle-on-a-chip bioconstructs with this drug under microgravity conditions for 7 days (FIG. 18A).

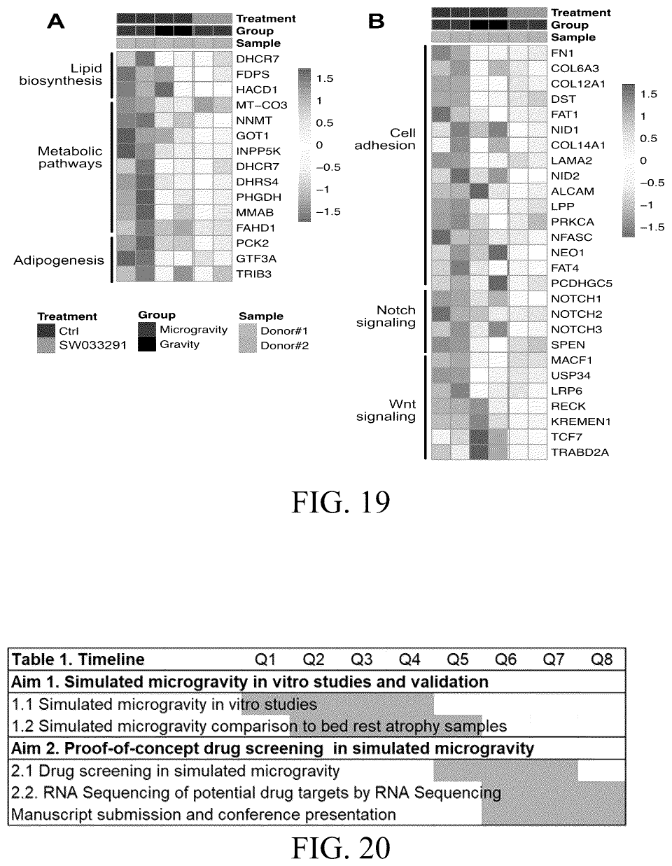

Our preliminary RNA sequencing data demonstrates that engineered muscle in microgravity that was treated with SW033291 more closely resembled gravity-like conditions with respect to lipid biosynthesis and metabolic pathways (FIG. 19). Moreover, numerous pathways whose downregulation is associated with atrophy were also downregulated with space flight, but then partially prevented with drug treatment (FIG. 19). This data provides compelling evidence that SW033291 can partially prevent aspects of muscle atrophy and therefore serves as a reference drug in the disclosed drug screening studies.

### H. Summary

In summary, these preliminary data demonstrate the feasibility of engineering muscle-on-a-chip constructs composed of human MPCs seeded on aligned nanofibrillar collagen scaffolds that direct the organization of myotube formation and promote synchronized contractility (FIGS. 14A-C). We further show that the muscle-on-a-chip bioconstructs can be housed within microgravity-compatible BioCell bioreactors (FIG. 3A-D). When launched to the ISSNL, the bioconstructs in microgravity showed salient aspects of muscle atrophy, based on RNA sequencing and morphological visualization of myotube formation. These studies provide compelling evidence that microgravity adversely affects myotube formation within the engineered muscles, suggesting that microgravity impairs myogenesis (FIGS. 5G, 15). Furthermore, to show the proof-of-concept of this platform to perform drug testing, we showed that SW033291 could partially prevent aspects of muscle atrophy, based on RNA sequencing (FIGS. 18A, 18B, 19). Together, these data show feasibility and substantiate our rationale for applying this established muscle-on-a-chip platform to develop and validate a more readily accessible simulated model of microgravity-through the use of a 3D clinostat for research use.

### I. Objective

Accordingly, the objective of this project is to develop and validate a novel in vitro model of disuse atrophy using engineered skeletal muscle under simulated microgravity conditions, and to apply this platform for pilot drug screening (FIG. 13). It is contemplated that validation studies as disclosed herein can characterize and optimize critical parameters of this system to enable more widespread use of this platform. Such parameters include the minimal required time in simulated microgravity for observing muscle unloading effects, along with the process development of a drug screening pipeline in simulated microgravity. The findings from this study are expected to increase the use of simulated microgravity to study muscle unloading, which affects a large population of Veterans.

### Research Design & Methods

A. Specific Aim 1. To quantitatively assess the efficacy of in vitro engineered skeletal muscle in simulated microgravity to recapitulate salient features of disuse muscle atrophy.

### 1) Approach.

Engineered human skeletal muscle can be cultured in simulated microgravity conditions within a 3D clinostat or under normal gravity conditions. Owing to the small size and mass of the engineered muscles, it is not feasible to quantify the change in muscle mass or volume change. Instead, quantitative assessment of surrogate output measures (i.e., muscle contractility, myotube formation/organization, and transcriptional profiling) can be performed for up to 14 days. The genomic signature of samples in microgravity can be compared to samples in normal gravity. Additionally, the data can be compared to RNASeq datasets derived from clinical muscle unloading studies, to validate this in vitro model of muscle atrophy.

### 2) Fabrication of Aligned Nanofibrillar Scaffolds.

The scaffolds can be produced by a facile method of extruding bovine monomeric collagen type I from a fine needle into a pH 7.4 buffer, forming collagen strips (500 μm thick) along the direction of extrusion (FIGS. 14A, 16) [23-26]. Importantly, extrusion of collagen at high speed can induce the formation of parallel-oriented nanofibrils with nanofibril diameters of 30-50 nm. By reducing the speed of extrusion, the fibrillogenesis becomes more disorganized, leading to the formation of randomly oriented nanofibrillar scaffolds. As shown by scanning electron microscopy (FIG. 14A), the aligned collagen fibrils are parallel-oriented, in comparison to the random distribution of collagen fibrils in the conventional randomly oriented scaffolds.

### 3) Engineered Skeletal Muscle Myogenesis in Microgravity.

Ten scaffold strips (each 2-cm-long, 0.5 mm wide) produced by collagen extrusion can be adhered onto a segment of glass (Schott H, Nexterion, 25×25 mm) that is surface modified to inhibit cell attachment onto glass. Each scaffold strip can be adhered in parallel to other strips and 0.5 mm apart. After disinfection using 70% ethanol, the scaffolds can be seeded with healthy primary human MPCs (10⁴ Pax7+cells/mm² derived from male and female vastus lateralis muscle, Lifeline Cell Technologies) and cultured in expansion growth media (StemLife Skeletal Media, Lifeline Cell Technologies) for two days (denoted as day −2 to day 0), as shown in FIG. 18B (n=5). As a reference control, MPCs can also be grown on collagen-coated glass coverslips for comparison to on collagen scaffolds.

4) Assembly into Bioreactors for Microgravity or Gravity Conditions.

On day 0 of the study protocol, the cell-seeded scaffolds can be assembled into the Biocell bioreactors (FIG. 3A) to allow for complete submerging of the muscle-on-a-chip samples within media. The media can be replenished with an induction medium consisting of Dulbecco's Modified Eagle's Medium (DMEM) and 5% horse serum to induce myotube formation. The BioCell bioreactors can be placed into the SciSpinner 3D clinostat and subjected to microgravity conditions (10⁻³ g) for up to 14 days. For comparison, the cell-seeded scaffolds can be assembled into bioreactors and then stored in conventional cell culture incubators at 37° C. and 5% O₂. Similar to samples in microgravity, the cell-seeded scaffolds in normal gravity conditions can be grown in expansion media for 2 days before switching to an induction media (FIG. 18B). At time points up to 14 days in induction media, the samples can be assayed for.

i. Myotube Formation and Organization.

To quantitatively assess the effect of microgravity on myotube formation, MPCs cultured in induction media can undergo myotube formation on nanofibrillar collagen scaffolds. At time points up to 14 days in induction media, the engineered muscle samples can be fixed in 4% paraformaldehyde for immunofluorescence staining of skeletal muscle myosin heavy chain (MyHC) (FIG. 14B) and MPC marker Pax7 using immunofluorescence staining procedures well-established in our laboratory [23, 24]. Using confocal microscopy, myotube length, myotube alignment, myotube formation, myotube striations, and the relative abundance of Pax7 expression can be compared between samples under gravity and microgravity, based on quantitative approaches established by our laboratory [32, 33]. Alignment of the myotubes can be quantified by measuring the angle formed by the direction of the myotubes with respect to the axis of the aligned nanofibrils [32, 33]. Myotube formation can be quantified by the proportion of nuclei incorporated into myotubes [33] as a fusion index (n=5). The number of Pax7⁺ cells can be expressed as a percentage of total cells. These analyses can quantitatively assess the influence of microgravity on myotube formation, alignment, and the preservation of Pax7⁺ niche within engineered tissues. These studies can demonstrate the temporal effects of simulated microgravity in regulating myogenesis based on myotube formation.

ii. Contractility.

Functional measurements of muscle contractility in response to electrical stimulation (Myopacer, Ionoptix) can be quantified at time points up to 14 days in induction media (n=5). Based on our previous protocols [11, 34], the myotubes can be stimulated at 1 Hz, pulse duration of 4 ms, and amplitude of 30 V. Videos of myotube contractility can be recorded at 10× objectives and processed using a custom Matlab code [35] that generates motion vector fields to compute contraction heatmaps and computed values of the maximum contraction velocity (μm s−1) and periodicity of contraction (expressed as contractions per minute). These studies can demonstrate the temporal kinetics of simulated microgravity in regulating myotube contractility [36, 37, 38].

iii. Transcriptional Profiling of Microgravity and Gravity Samples.

To determine whether engineered muscle in microgravity bears a signature that mimics muscle atrophy, we can perform RNA sequencing. At time points up to 14 days in induction media, the engineered skeletal muscle in gravity or simulated microgravity conditions can be lysed. Total RNA can be converted to cDNA, which can be further fragmented to an average of 300 bps, and sequencing adapters can be ligated to cDNA. PCR can be performed on the adapter-ligated cDNA. Libraries can be submitted for sequencing using paired in reads at an average length of 100 bps. Reads can be mapped with Tophat 2.0.8b using reference annotation (GRCh38 version 76) [39]. Cuffcompare and Cuffdiff can be used to determine which gene levels are significantly different (q<0.05). Only transcripts uniquely mapped to genes can be retained. The read-counts for multiple transcripts mapped to the same genes can be aggregated by summation at a linear-scale. Gene level reads-counts can be log 2 transformed with pseudo-count of one. In some examples, only protein coding genes will be included and gene expression will be quantile normalized for analysis. To identify functionally important transcription factors, gene function classes, and pathways that correspond to differences between treatment groups, enrichment analyses of the top perturbed genes can be performed against several gene annotation sets. Pathway analysis (Path Finder) can determine key signaling pathways and driver genes that mediate muscle formation and survival [40, 41, 42, 43, 44, 45].

iv. Transcriptional Comparison between Engineered Skeletal Muscle and Clinical Samples.

To determine whether the genomic profile of engineered muscle in microgravity resembles that of muscle biopsies from individuals with unloading/disuse muscle atrophy, we can compare the differentially expressed genes with those associated with muscle function and atrophy. The differentially expressed genes in microgravity (relative to those in gravity) can be compared to differentially expressed genes from clinical muscle atrophy tissue (relative to healthy muscle). The RNA Seq dataset from clinical samples can be derived from vastus lateralis muscle (n=9) from subjects undergoing bed rest with a −6° head down tilt for 35 days. The RNA Seq dataset is accessible for the subjects before (healthy) and after bed rest-induced atrophy through accession number GSE186045 [46].

The differentially expressed genes in microgravity can be compared to genes associated with muscle atrophy, including those involved in mitochondrial function and ATP synthesis (ie., ATP5D, ATP5G1) and gene transcription (ie., RPLP0, EEF1A2) [46, 47]. Another gene of interest is peroxisome proliferator-activated receptor-γ coactivator-1α (PGC-1α), which transcriptionally regulates muscle mass and function and is known to be differentially expressed during muscle disuse [48]. Based on the heat map of differentially expressed genes, we can determine whether samples in microgravity share a similar genomic signature as muscle samples from individuals with bed-rest associated atrophy. We can use GO and KEGG analysis to examine the differentially expressed genes in biological interaction networks. This analysis can enable us to define the direct or indirect interactions among differentially expressed genes and to determine how closely the engineered muscle in microgravity platform mirrors the signaling pathways associated with unloading/disuse atrophy. Furthermore, these studies can determine the minimal duration of microgravity for inducing atrophy-like changes.

v. qPCR Validation of RNA Sequencing Data.

Findings from RNA sequencing can be confirmed by qPCR, which can be performed using standard protocols based on our previous publications [34, 49]. PCR primers can be selected from the top 10 most differentially expressed genes based on RNA Seq data. Gene expression data from qRT-PCR can be analyzed by the ΔΔC_{T} method from our previous publications [34, 49].

### 5) Anticipated Results & Alternatives.

Based on our own preliminary data derived from real microgravity conditions, along with published findings showing that spaceflight is known to cause muscle atrophy and muscle loss in microgravity [50], we anticipate that our engineered skeletal muscle platform in simulated microgravity can similarly impair muscle behavior and function, based on impaired myotube formation, reduced contractility, and a genomic profile that shows impaired myogenesis and function, compared to samples under gravity treatment. With respect to the kinetics of this decline, we anticipate that the time course studies can reveal the minimal time of simulated microgravity needed to induce these changes. Based on our preliminary data derived from real microgravity in which 7 days was sufficient to detect notable transcriptional changes (FIG. 5G), it is contemplated that the minimum duration of simulated microgravity can be no more than 7 days. We do recognize that our preliminary data derived generated aboard the ISSNL may differ somewhat from simulated microgravity, since radiation effects could also contribute to the findings derived from our studies aboard the ISSNL. As an alternative to the clinical dataset derived from bed rest for 35 days (GSE186045 [46]), we can also compare our in vitro RNA sequencing data to other accessible datasets that can be derived from vastus lateralis muscle biopsies from subjects subjected to bed rest atrophy (i.e., GSE113165).

### B. Specific Aim 2. To Apply the Engineered Skeletal Muscle in Simulated Microgravity Platform for Pilot Testing Drugs for Treatment of Muscle Atrophy.

### 1) Approach.

Engineered skeletal muscle in simulated microgravity can be screened with a collection of FDA-approved repurposed drugs for up to 14 days (FIGS. 18A, 18B). Initial studies can be performed to identify a range in drug concentration that is non-cytotoxic. In the setting of simulated microgravity, the drugs can be added to the media of the engineered muscles at the start of induction media (day 0) and maintained for up to 14 days. At each time point, immunofluorescence staining of MyHC and quantification of myotube formation and muscle contractility. Drugs showing >30% improvement in these output measures in simulated microgravity can be further examined by RNA sequencing to reveal the mechanism by which the drugs act. A reference compound can also be included, namely SW033291 that has shown benefit in our preliminary in vitro studies in microgravity, as well as in preclinical studies for treatment of aging related muscle atrophy [12].

### 2) Initial Drug Dosing Studies.

Using the validated muscle-on-a-chip in simulated microgravity platform, we can screen a library of FDA-approved drugs (FDA-Approved Drug Library, Tocriscreen) for repurposing. The Tocriscreen collection of compounds is equivalent to the pharmacologically active components of drug formulations approved by the FDA. The collection covers a range of known therapeutic applications, including musculoskeletal, cancer, and cardiovascular areas. The compounds have been utilized in other disease applications [51, 52], but not yet for treatment of muscle atrophy. We can first perform ground drug screening studies by testing a suitable range of drug that does not show cytotoxic effects. To perform this analysis in a feasible way, 103 MPCs can be initially directly within 384 well dishes to confluency overnight before initiating differentiation with the induction media. To show proof-of-concept of this drug screening platform, we can select 12 compounds from the compound library, based on known indications for musculoskeletal or cardiac applications. The 12 compounds can be diluted into the induction media at a range of concentrations (0.1-1000 μM) before adding to the cultured cells. Over the course of 7 days in induction media, the cells can be evaluated for cytotoxicity based on a rounded or non-adherent cellular morphology, and then verified using the Live/Dead Cytotoxicity assay (Fisher Scientific). The success criteria to proceed with simulated microgravity studies consist of drug concentrations that show no evidence of cytotoxicity (>80% viability, n=5).

### 3) Drug Screening Studies in Microgravity.

Engineered muscle-on-a-chip constructs can be fabricated and assembled into bioreactors as described above. Working concentrations of drugs that pass the success criteria of showing >80% cell viability can be added into the media for up to 14 days of simulated microgravity conditions (FIG. 18B). The compound SW033291 (200 nM) can be included as a reference compound with therapeutic potential, based on our prior ISSNL study, but can be validated here in simulated microgravity [12]. Negative control samples can be subjected to microgravity and treated with the vehicle control (dimethylsulfoxide, DMSO). Parallel experiments can be conducted using muscle-on-a-chip bioconstructs in normal gravity conditions. To study the effect of each drug in microgravity condition, we can compare the degree of myotube formation and contractility to samples in microgravity+ DMSO control addition. The success criteria for identifying a potential drug target for preventing atrophy-like features are: an improvement by >25% in contractility and myotube formation capability, compared to the samples treated with control DMSO. The drug compounds that meet this success criteria can then be examined by RNA sequencing to reveal the underlying mechanism by which the drugs act [53-55].

### 4) Anticipated Results & Limitations.

Output measures consist of quantitative comparison of the proteomic expression of engineered muscle exposed to drug compounds in microgravity, compared to the vehicle control in microgravity, and also in comparison to the corresponding experiments in gravity conditions. The success criteria of the disclosed studies include identifying putative drug targets that prevent atrophy-like qualities in microgravity conditions, based on >25% improvement in contractility and myotube formation, in comparison to samples in microgravity exposed to control DMSO. It is likely that SW033291 would show benefit, based on our preliminary data derived from real microgravity studies. For this reason, this drug can be used as a reference control to the 12 drug compounds. We expect that a promising drug compound could prevent atrophy-like qualities by shifting the transcriptomic signature away from clinical atrophy samples and closer to healthy clinical muscle samples. Since only 12 drug compounds are tested here for proof-of-concept, if none show significant benefit, we can expand the drug screening to 30 compounds. We do recognize that primary human MPCs have limited expansion capability, which may limit the number of experiments that can be performed for each donor line. It is contemplated that MPCs from multiple donors can be pooled to maintain a larger bank, although doing so may increase heterogeneity of the cell population. Another alternative is to use the HMCL-7304 immortalized MPC line, as it is well-characterized with unlimited expansion capacity [56].

### 5) Statistical Analysis & Rigor.

Output measures for comparison between gravity and simulated microgravity conditions consist of quantitative assessment of myotube formation, organization, contractility, and RNA sequencing. The sample size (n=5) for the microgravity studies was chosen based on power calculations to achieve >0.8 power. For quantification of myotube formation, myotube morphology, secreted cytokine arrays, and qPCR, statistical significance between three or more treatment groups can be accepted at P<0.05 by analysis of variance (ANOVA) with Bonferroni post-test. For RNA sequencing, statistical significances can be adjusted by the Benjamini-Hochberg False Discovery Rate multiple-testing correction set at a threshold of 5%. Data collection and analysis can be performed in a blinded fashion to ensure scientific rigor. Since sex is a biological variable, it is contemplated that both male and female MPCs can be studied. FIG. 20 shows the timeline of the disclosed studies.

The following references of Example 3 are incorporated by reference herein in their respective entireties:
[1] Di Girolamo F G, Fiotti N, Milanovic Z, Situlin R, Mearelli F, Vinci P, Simunic B, Pisot R, Narici M, Biolo G. The aging muscle in experimental bed rest: A systematic review and meta-analysis. Front Nutr 2021; 8633987. PMC8371327.
[2] Hirsch C H, Sommers L, Olsen A, Mullen L, Winograd C H. The natural history of functional morbidity in hospitalized older patients. J Am Geriatr Soc 1990; 38(12):1296-1303.
[3] Zisberg A, Shadmi E, Sinoff G, Gur-Yaish N, Srulovici E, Admi H. Low mobility during hospitalization and functional decline in older adults. J Am Geriatr Soc 2011; 59(2):266-273.
[4] Fortinsky R H, Covinsky K E, Palmer R M, Landefeld C S. Effects of functional status changes before and during hospitalization on nursing home admission of older adults. Journals of Gerontology Series A, Biological Sciences & Medical Sciences 1999; 54(10):M521-526.
[5] Fitts R H, Trappe S W, Costill D L, Gallagher P M, Creer A C, Colloton P A, Peters J R, Romatowski J G, Bain J L, Riley D A. Prolonged space flight-induced alterations in the structure and function of human skeletal muscle fibres. J Physiol 2010; 588(Pt 18):3567-3592. PMC2988519.
[6] Trappe T. Influence of aging and long-term unloading on the structure and function of human skeletal muscle. Appl Physiol Nutr Metab 2009; 34(3):459-464. PMC3056056.
[7] Takahashi H, Nakamura A, Shimizu T. Simulated microgravity accelerates aging of human skeletal muscle myoblasts at the single cell level. Biochemical & Biophysical Research Communications 2021; 578115-121.
[8] Smith R C, Cramer M S, Mitchell P J, Lucchesi J, Ortega A M, Livingston E W, Ballard D, Zhang L, Hanson J, Barton K, Berens S, Credille K M, Bateman T A, Ferguson V L, Ma Y L, Stodieck L S. Inhibition of myostatin prevents microgravity-induced loss of skeletal muscle mass and strength. PLoS One 2020; 15(4):e0230818. PMC7173869.
[9] Furukawa T, Tanimoto K, Fukazawa T, Imura T, Kawahara Y, Yuge L. Simulated microgravity attenuates myogenic differentiation via epigenetic regulations. NPJ Microgravity 2018; 4:11. PMC5966377.
[10] Mitchell P O, Pavlath G K. Skeletal muscle atrophy leads to loss and dysfunction of muscle precursor cells. Am J Physiol Cell Physiol 2004; 287(6):C1753-1762.
[11] Nakayama K H, Quarta M, Paine P, Alcazar C, Karakikes I, Garcia V, Abilez O J, Calvo N S, Simmons C S, Rando T A, Huang N F. Treatment of volumetric muscle loss in mice using nanofibrillar scaffolds enhances vascular organization and integration. Commun Biol 2019; 2:170. PMC6505043.
[12] Palla A R, Ravichandran M, Wang Y X, Alexandrova L, Yang A V, Kraft P, Holbrook C A, Schurch C M, Ho A T V, Blau H M. Inhibition of prostaglandin-degrading enzyme 15-pgdh rejuvenates aged muscle mass and strength. Science 2021; 371(6528):abc8059. PMC7938328.
[13] Kilroe S P, Fulford J, Jackman S, Holwerda A, Gijsen A, van Loon L, Wall B T. Dietary protein intake does not modulate daily myofibrillar protein synthesis rates or loss of muscle mass and function during short-term immobilization in young men: A randomized controlled trial. Am J Clin Nutr 2021; 113(3):548-561.
[14] Trappe T A, Burd N A, Louis E S, Lee G A, Trappe S W. Influence of concurrent exercise or nutrition countermeasures on thigh and calf muscle size and function during 60 days of bed rest in women. Acta Physiol (Oxf) 2007; 191(2):147-159.
[15] Kortebein P, Symons T B, Ferrando A, Paddon-Jones D, Ronsen O, Protas E, Conger S, Lombeida J, Wolfe R, Evans W J. Functional impact of 10 days of bed rest in healthy older adults. Journals of Gerontology Series A, Biological Sciences & Medical Sciences 2008; 63(10):1076-1081.
[16] Ferrando A A, Paddon-Jones D, Hays N P, Kortebein P, Ronsen O, Williams R H, McComb A, Symons T B, Wolfe R R, Evans W. Eaa supplementation to increase nitrogen intake improves muscle function during bed rest in the elderly. Clin Nutr 2010; 29(1):18-23.
[17] Murgia M, Ciciliot S, Nagaraj N, Reggiani C, Schiaffino S, Franchi M V, Pisot R, Simunic B, Toniolo L, Blaauw B, Sandri M, Biolo G, Fluck M, Narici M V, Mann M. Signatures of muscle disuse in spaceflight and bed rest revealed by single muscle fiber proteomics. PNAS Nexus 2022; 1(3):pgac086. PMC9896895.
[18] Barzegari A, Saei A A. An update to space biomedical research: Tissue engineering in microgravity bioreactors. Bioimpacts 2012; 2(1):23-32. PMC3648913.
[19] Manzano A, Herranz R, den Toom L A, Te Slaa S, Borst G, Visser M, Medina F J, van Loon J. Novel, moon and mars, partial gravity simulation paradigms and their effects on the balance between cell growth and cell proliferation during early plant development. NPJ Microgravity 2018; 4:9. PMC5884789 systems. The remaining authors declare that they have no competing interests.
[20] Zhang Y, Richards J T, Hellein J L, Johnson C M, Woodall J, Sorenson T, Neelam S, Ruby A M J, Levine H G. Nasa's ground-based microgravity simulation facility. Methods Mol Biol 2022; 2368:281-299.
[21] Clary J L, France C S, Lind K, Shi R, Alexander J S, Richards J T, Scott R S, Wang J, Lu X-H, Harrison L. Development of an inexpensive 3d clinostat and comparison with other microgravity simulators using mycobacterium marinum Front Space Technol 2022; 3:1032610.
[22] Bentzinger C F, Wang Y X, Dumont N A, Rudnicki M A. Cellular dynamics in the muscle satellite cell niche. EMBO Rep 2013; 14(12):1062-1072. PMC3849491.
[23] Nakayama K H, Hong G, Lee J C, Patel J, Edwards B, Zaitseva T S, Paukshto M V, Dai H, Cooke J P, Woo Y J, Huang N F. Aligned-braided nanofibrillar scaffold with endothelial cells enhances arteriogenesis. ACS Nano 2015; 9(7):6900-6908. PMC4757475.
[24] Nakayama K H, Surya V N, Gole M, Walker T W, Yang W, Lai E S, Ostrowski M A, Fuller G G, Dunn A R, Huang N F. Nanoscale patterning of extracellular matrix alters endothelial function under shear stress. Nano Lett 2016; 16(1):410-419. PMC4758680.
[25] Nakayama K H, Joshi P A, Lai E S, Gujar P, Joubert L M, Chen B, Huang N F. Bilayered vascular graft derived from human induced pluripotent stem cells with biomimetic structure and function. Regen Med 2015; 10(6):745-755. PMC4760352.
[26] Lai E S, Anderson C M, Fuller G G. Designing a tubular matrix of oriented collagen fibrils for tissue engineering. Acta Biomater 2011; 7(6):2448-2456.
[27] Yartseva V, Goldstein L D, Rodman J, Kates L, Chen M Z, Chen Y J, Foreman O, Siebel C W, Modrusan Z, Peterson A S, Jovicic A. Heterogeneity of satellite cells implicates delta1/notch2 signaling in self-renewal. Cell Rep 2020; 30(5):1491-1503 e1496.
[28] Punetha J, Kesari A, Hoffman E P, Gos M, Kaminska A, Kostera-Pruszczyk A, Hausmanowa-Petrusewicz I, Hu Y, Zou Y, Bonnemann C G, M J E. Novel coll2al variant expands the clinical picture of congenital myopathies with extracellular matrix defects. Muscle & Nerve 2017; 55(2):277-281. PMC5236000.
[29] Martinez J R, Dhawan A, Farach-Carson M C. Modular proteoglycan perlecan/hspg2: Mutations, phenotypes, and functions. Genes (Basel) 2018; 9(11):556. PMC6266596.
[30] Ho A T V, Palla A R, Blake M R, Yucel N D, Wang Y X, Magnusson K E G, Holbrook C A, Kraft P E, Delp S L, Blau H M. Prostaglandin e2 is essential for efficacious skeletal muscle stem-cell function, augmenting regeneration and strength. Proc Natl Acad Sci USA 2017; 114(26):6675-6684. PMC5495271.
[31] Mo C, Zhao R, Vallejo J, Igwe O, Bonewald L, Wetmore L, Brotto M. Prostaglandin e2 promotes proliferation of skeletal muscle myoblasts via ep4 receptor activation. Cell Cycle 2015; 14(10):1507-1516. PMC4615122.
[32] Huang N F, Lee R J, Li S. Engineering of aligned skeletal muscle by micropatterning. Am J Transl Res 2010; 2(1):43-55. PMC2826821.
[33] Huang N F, Patel S, Thakar R G, Wu J, Hsiao B S, Chu B, Lee R J, Li S. Myotube assembly on nanofibrous and micropatterned polymers. Nano Lett 2006; 6(3):537-542.
[34] Wanjare M, Hou L, Nakayama K H, Kim J J, Mezak N P, Abilez O J, Tzatzalos E, Wu J C, Huang N F. Anisotropic microfibrous scaffolds enhance the organization and function of cardiomyocytes derived from induced pluripotent stem cells. Biomater Sci 2017; 5(8):1567-1578. PMC5567776.
[35] Huebsch N, Loskill P, Mandegar M A, Marks N C, Sheehan A S, Ma Z, Mathur A, Nguyen T N, Yoo J C, Judge L M, Spencer C I, Chukka A C, Russell C R, So P L, Conklin B R, Healy K E. Automated video-based analysis of contractility and calcium flux in human-induced pluripotent stem cell-derived cardiomyocytes cultured over different spatial scales. Tissue Eng Part C Methods 2015; 21(5):467-479. PMC4410286.
[36] Leeper N J, Myers J, Zhou M, Nead K T, Syed A, Kojima Y, Caceres R D, Cooke J P. Exercise capacity is the strongest predictor of mortality in patients with peripheral arterial disease. J Vase Surg 2013; 57(3):728-733. PMC3543469.
[37] Chang P, Nead K T, Olin J W, Myers J, Cooke J P, Leeper N J. Effect of physical activity assessment on prognostication for peripheral artery disease and mortality. Mayo Clin Proc 2015; 90(3):339-345.
[38] Liang G, Huang X, Hirsch J, Mehmi S, Fonda H, Chan K, Huang N F, Aalami O, Froelicher V F, Lee D P, Myers J, Lee A S, Nguyen P K. Modest gains after an 8-week exercise program correlate with reductions in non-traditional markers of cardiovascular risk. Front Cardiovasc Med 2021; 8:669110. PMC8245677.
[39] Guo Y, Dai Y, Yu H, Zhao S, Samuels D C, Shyr Y. Improvements and impacts of grch38 human reference on high throughput sequencing data analysis. Genomics 2017; 109(2):83-90.
[40] Huang N F, Lai E S, Ribeiro A J, Pan S, Pruitt B L, Fuller G G, Cooke J P. Spatial patterning of endothelium modulates cell morphology, adhesiveness and transcriptional signature. Biomaterials 2013; 34(12):2928-2937. PMC3581686.
[41] Li J, Huang N F, Zou J, Laurent T J, Lee J C, Okogbaa J, Cooke J P, Ding S. Conversion of human fibroblasts to functional endothelial cells by defined factors. Arteriosclerosis, Thrombosis, & Vascular Biology 2013; 33(6):1366-1375. PMC3898631.
[42] Conboy I M, Conboy M J, Smythe G M, Rando T A. Notch-mediated restoration of regenerative potential to aged muscle. Science 2003; 302(5650):1575-1577.
[43] Rando T A, Chang H Y. Aging, rejuvenation, and epigenetic reprogramming: Resetting the aging clock. Cell 2012; 148(1-2):46-57. PMC3336960.
[44] Nakayama K H, Alcazar C, Yang G, Quarta M, Paine P, Doan L, Davies A, Rando T A, Huang N F. Rehabilitative exercise and spatially patterned nanofibrillar scaffolds enhance vascularization and innervation following volumetric muscle loss. NPJ Regen Med 2018; 3:16. PMC6141593.
[45] Hu C, Ayan B, Chiang G, Chan A H P, Rando T A, Huang N F. Comparative effects of basic fibroblast growth factor delivery or voluntary exercise on muscle regeneration after volumetric muscle loss. Bioengineering (Basel) 2022; 9(1):37. PMC8773127.
[46] McFarland A J, Ray P R, Bhai S, Levine B D, Price T J. Rna sequencing on muscle biopsy from a 5-week bed rest study reveals the effect of exercise and potential interactions with dorsal root ganglion neurons. Physiol Rep 2022; 10(3):e15176. PMC8823189.
[47] Liu D, Sartor M A, Nader G A, Pistilli E E, Tanton L, Lilly C, Gutmann L, IglayReger H B, Visich P S, Hoffman E P, Gordon P M. Microarray analysis reveals novel features of the muscle aging process in men and women. Journals of Gerontology Series A, Biological Sciences & Medical Sciences 2013; 68(9):1035-1044. PMC3826860.
[48] Buso A, Comelli M, Picco R, Isola M, Magnesa B, Pisot R, Rittweger J, Salvadego D, Simunic B, Grassi B, Mavelli I. Mitochondrial adaptations in elderly and young men skeletal muscle following 2 weeks of bed rest and rehabilitation. Front Physiol 2019; 10:474. PMC6504794.
[49] Huang N F, Fleissner F, Sun J, Cooke J P. Role of nitric oxide signaling in endothelial differentiation of embryonic stem cells. Stem Cells Dev 2010; 19(10):1617-1626. PMC3121801.
[50] Fitts R H, Riley D R, Widrick J J. Physiology of a microgravity environment invited review: Microgravity and skeletal muscle. J Appl Physiol (1985) 2000; 89(2):823-839.
[51] Mehyar N, Mashhour A, Islam I, Gul S, Adedeji A O, Askar A S, Boudjelal M. Using in silico modelling and fret-based assays in the discovery of novel fda-approved drugs as inhibitors of mers-cov helicase. SAR & QSAR in Environmental Research 2021; 32(1):51-70.
[52] Shin J S, Jung E, Kim M, Baric R S, Go Y Y. Saracatinib inhibits middle east respiratory syndrome-coronavirus replication in vitro. Viruses 2018; 10(6):283. PMC6024778.
[53] Wnorowski A, Sharma A, Chen H, Wu H, Shao N Y, Sayed N, Liu C, Countryman S, Stodieck L S, Rubins K H, Wu S M, Lee P H U, Wu J C. Effects of spaceflight on human induced pluripotent stem cell-derived cardiomyocyte structure and function. Stem Cell Reports 2019; 13(6):960-969. PMC6915842.
[54] Liang P, Lan F, Lee A S, Gong T, Sanchez-Freire V, Wang Y, Diecke S, Sallam K, Knowles J W, Wang P J, Nguyen P K, Bers D M, Robbins R C, Wu J C. Drug screening using a library of human induced pluripotent stem cell-derived cardiomyocytes reveals disease-specific patterns of cardiotoxicity. Circulation 2013; 127(16):1677-1691. PMC3870148.
[55] Sayed N, Liu C, Ameen M, Himmati F, Zhang J Z, Khanamiri S, Moonen J R, Wnorowski A, Cheng L, Rhee J W, Gaddam S, Wang K C, Sallam K, Boyd J H, Woo Y J, Rabinovitch M, Wu J C. Clinical trial in a dish using ipscs shows lovastatin improves endothelial dysfunction and cellular cross-talk in lmna cardiomyopathy. Sci Transl Med 2020; 12(554):eaax9276. PMC7557117.
[56] Rokach O, Ullrich N D, Rausch M, Mouly V, Zhou H, Muntoni F, Zorzato F, Treves S. Establishment of a human skeletal muscle-derived cell line: Biochemical, cellular and electrophysiological characterization. Biochem J 2013; 455(2):169-177.

### Example 4

### SUMMARY

Determining Key Drugs Compounds that Reverse Sarcopenia-Like Characteristics of the HMCLs in a 2D Simulated Microgravity Environment.

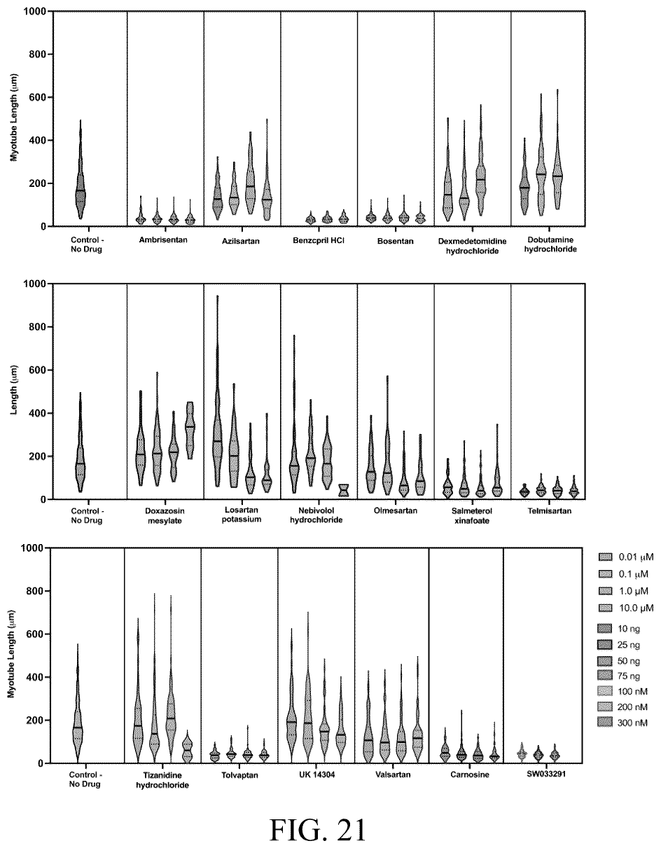

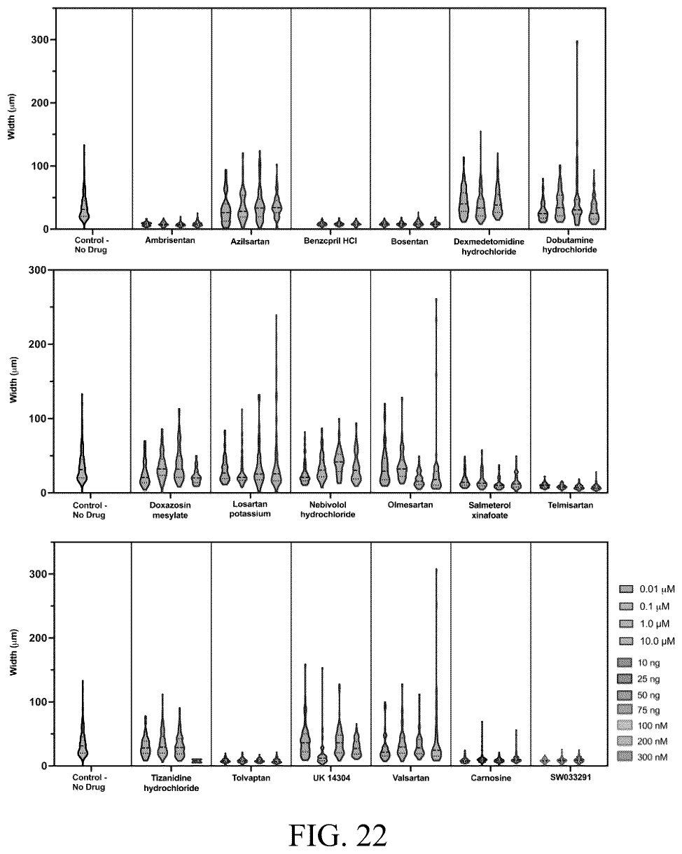

Currently, an optimal format for our studies uses a 96 well plate, where up to 8 samples can be studied at 4 different concentrations in triplicate. Human Muscle Cell Line (HMCLs) were grown in Promocell Media (Cat. No. C-23260)+Skeletal Muscle kit (Cat. No. C-39360)+1% PS (Gibco 15140-122) and plated at high density (>95% confluency) into 96 well plates. After overnight adherence the cells were switched to differentiation media (DMEM (Gibco #11995065)+5% HS (Gibco #16-050-122)) and treated with varying concentrations of the compounds of interest. The 96 well plates were covered in Breathe Easy Sealing Film (Thomas Scientific #T796200) and placed in the Clinostat at 1.7 RMP for the entire differentiation period. The media and drugs were changed and replenished daily. As we are interested in determining the effects of these compounds on skeletal muscle regeneration, any positive characteristics that are seen to increase myotube formation must be noted. To ensure myotube presence and visualize morphological differences the samples are stained for Myosin Heavy Chain (MHC) and Hoechst before fluorescent images are acquired. Representative images of drugs showing varying effect when compared to the control can be found in FIG. 11. Through this setup, multiple drugs were screened and analyzed using the metrics myotube length (FIG. 21) and myotube width (FIG. 22).

Comparing Effects of Simulated Microgravity Vs. Normal Gravity Conditions on Myotube Morphology on Collagen Nanopatterned Scaffolds.

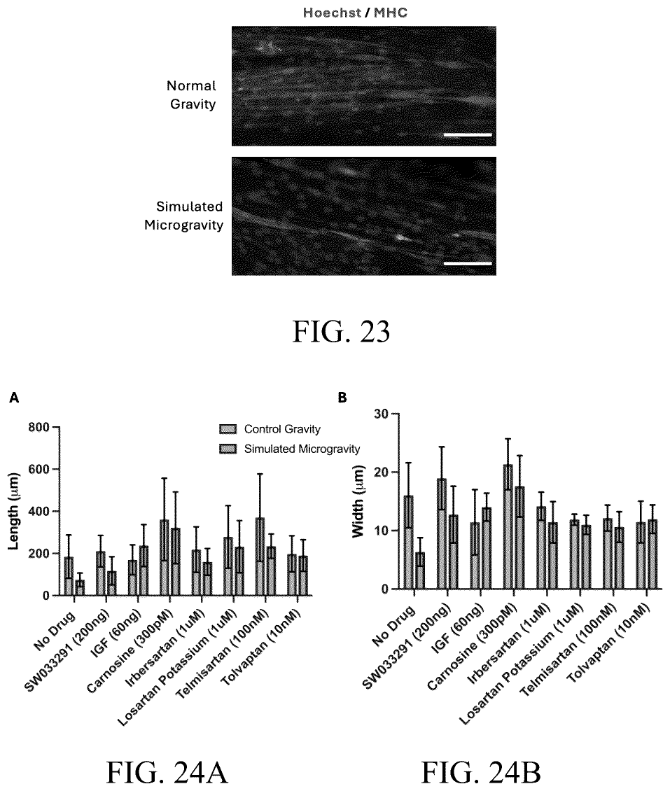

Experiments were performed comparing the morphological effects on myotube formation between normal gravity conditions and our simulated microgravity system. Nanopatterned collagen scaffolds were prepared under previously published methodologies (Nakayama, 2019). C2C12 cells were seeded at high density (600,000 cells/scaffold) and allowed to adhere overnight. After overnight adherence the cells were switched to differentiation media (DMEM (Gibco #11995065)+5% HS (Gibco #16-050-122)). The Biocells were placed wither in the clinostat at 1.7 RPM or were allowed to remain under static conditions for the entire differentiation period. The media was changed and replenished daily. The samples were stained for Myosin Heavy Chain (MHC) and Hoechst before fluorescent images are acquired. Representative images of normal gravity conditions when compared to simulated microgravity can be found in FIG. 23. Additional experiments exploring the variances among the compound of interest under normal gravity and simulated microgravity conditions were also performed. Human Muscle Cell Line (HMCLs) were grown in Promocell Media (Cat. No. C-23260)+Skeletal Muscle kit (Cat. No. C-39360)+1% PS (Gibco 15140-122) and seeded at high density (750,000 cells/scaffold) and allowed to adhere overnight. After overnight adherence the cells were switched to differentiation media (DMEM (Gibco #11995065)+5% HS (Gibco #16-050-122)) and treated with varying concentrations of the compounds of interest. Through this setup, multiple drugs were screened and analyzed using the metrics myotube length (FIG. 24A) and myotube width (FIG. 24B).

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A method for screening a test substance affecting myogenesis, the method comprising:
a) providing an engineered muscle tissue, wherein the engineered muscle tissue has been exposed to microgravity conditions;
b) contacting the engineered muscle tissue with the test substance;
c) determining the expression of at least one mitochondrion gene or at least one biological process gene in the engineered muscle tissue after step b);
d) comparing the expression of the at least one mitochondrion gene or the at least one biological process gene in the engineered muscle tissue in step b) to the expression of the at least one mitochondrion gene or the at least one biological process gene in the engineered muscle tissue prior to step b),
wherein a decrease in expression of the at least one mitochondrion gene indicates that the test substance increases myogenesis or wherein an increase in the expression of the at least one biological process gene indicates that the test substance increases myogenesis.

Aspect 2: A method for identifying a test substance that increases muscle myogenesis, the method comprising:
a) contacting engineered muscle tissue with the test substance, wherein the engineered muscle tissue has been exposed to microgravity conditions prior to contacting the engineered muscle tissue with the test substance;
b) determining the expression of at least one mitochondrion gene or at least one biological process gene in the engineered muscle tissue after the engineered muscle tissue is contacted with the test substance; and
c) comparing the expression of the at least one mitochondrion gene or the at least one biological process gene in the engineered muscle tissue in step b) to the expression of the at least one mitochondrion gene or the at least one biological process gene in the engineered muscle tissue prior to step a),
wherein a decrease in expression of the at least one mitochondrion gene indicates that the test substance increases myogenesis or wherein an increase in the expression of the at least one biological process gene indicates that the test substance increases myogenesis.

Aspect 3: A method for screening a test substance affecting myogenesis, the method comprising:
a) providing an engineered muscle tissue, wherein the engineered muscle tissue has been exposed to microgravity conditions;
b) contacting the engineered muscle tissue with the test substance;
c) immunofluorescently staining myosin heavy chain in the engineered muscle tissue, wherein the engineered muscle tissue comprises myotubes; and
d) measuring myotube length or myotube width in the engineered muscle tissue via the expression of the myosin heavy chain in the myotubes,
wherein an increase in myotube length or myotube width compared to the myotube length or myotube width prior to contacting the engineered muscle tissue with the test substance indicates that the test substance increases myogenesis.

Aspect 4: A method for identifying a test substance that increases muscle myogenesis, the method comprising:
a) contacting engineered muscle tissue with the test substance, wherein the engineered muscle tissue has been exposed to microgravity conditions prior to contacting the engineered muscle tissue with the test substance;
b) contacting the engineered muscle tissue with the test substance;
c) immunofluorescently staining myosin heavy chain in the engineered muscle tissue, wherein the engineered muscle tissue comprises myotubes; and
d) measuring myotube length or myotube width in the engineered muscle tissue via the expression of the myosin heavy chain in the myotubes,
wherein an increase in myotube length or myotube width compared to the myotube length or myotube width prior to contacting the engineered muscle tissue with the test substance indicates that the test substance increases myogenesis

Aspect 5: The method of any of one of the preceding aspects, wherein the engineered muscle tissue is engineered skeletal muscle tissue.

Aspect 6: The method of any of one of the preceding aspects, wherein the engineered muscle tissue comprises a plurality of myoblasts.

Aspect 7: The method of any of one of the preceding aspects, wherein the microgravity conditions comprise exposing the engineered muscle tissue to 10-3 g for 7 to 14 days.

Aspect 8: The method of any of one of the preceding aspects, wherein the test substance is applied to the engineered muscle tissue for 7 to 14 days.

Aspect 9: The method of any one of aspects 1 or 2, wherein the at least one mitochondrion gene is selected from the group of mitochondrially encoded cytochrome c oxidase III (MT-CO3), fumarylacetoacetate hydrolase domain-containing protein 1 (FAHD1), lon protease homolog (LONP1), phosphoenolpyruvate carboxykinase 2 (PCK2), glutaredoxin-1 (GLRX), RAB32, Bcl-2 related ovarian killer (BOK), cytochrome C oxidase assembly factor 4 homolog (COA4), dehydrogenase/reductase 4 (DHRS4), fatty acid desaturase 1 (FADS1), EF-hand domain family member D1 (EFHD1), NX4, MTHFD2, and serum/glucocorticoid regulated kinase 1 (SGK1).

Aspect 10: The method of any one of aspects 1 or 2, wherein the at least one biological process gene is selected from the group consisting of protocadherin gamma C3 (PDCHGC3), prostaglandin-endoperoxide synthase 1 (PTGS1), transforming growth factor beta induced (TGFB1), NYN domain and retroviral integrase containing (NYNRIN), neuroligin 2 (NLGN2), hemicentin-1 (HMCN1), dystonin (DST), zinc finger and BTB domain-containing 20 (ZBTB20), laminin alpha 2 (LAMA2), collagen type XII alpha 1 chain (COL12A1), proline rich coiled-coil 2C (PRRC2C), nuclear factor of activated T-cells 5 (NFAT5), golgin subfamily B member 1 (GOLGB1), SCUBE3, zinc finger protein 469 (ZNF469), heparan sulfate proteoglycan 2 (HSPG2), PH and SEC7 domain-containing protein 3 (PSD3), cluster of differentiation 109 (CD109), neuroblast differentiation-associated protein AHNAK (AHNAK), and neurogenic locus notch homolog protein 2 (NOTCH2).

Aspect 11: The method of any one of the preceding aspects, further comprising performing a proteomic analysis of the engineered muscle tissue.

Aspect 12: The method of aspect 11, wherein the proteomic analysis comprises determining the amount of at least one protein selected from the group consisting of Eotaxin-3 (CCL26), C-X-C motif chemokine ligand 16 (CXCL16), growth differentiation factor 15 (GDF-15), tumor necrosis factor superfamily member 14 (LIGHT/TNSF14), and pupoid fetus (PF), wherein when the amount of Eotaxin-3 (CCL26), C-X-C motif chemokine ligand 16 (CXCL16), growth differentiation factor 15 (GDF-15), tumor necrosis factor superfamily member 14 (LIGHT/TNSF14), or pupoid fetus (PF) is decreased compared to the amount of the same protein present prior to contacting the engineered muscle tissue with the test substance indicates that the test substance increases myogenesis.

Aspect 13: The method of aspect 11, wherein the proteomic analysis comprises determining the amount of at least one protein selected from the group consisting of bone morphogenetic protein 4 (BMP-4), Resistin, C-X-C motif chemokine ligand 12 (CXCL12/SDF-1b), interleukin-16 (IL-16), and CD40, wherein when the amount of bone morphogenetic protein 4 (BMP-4), Resistin, C-X-C motif chemokine ligand 12 (CXCL12/SDF-1b), interleukin-16 (IL-16), or CD40 is increased compared to the amount of the same protein present prior to contacting the engineered muscle tissue with the test substance indicates that the test substance increases myogenesis.

Aspect 14: The method of any one of aspects 1 or 2, further comprising immunofluorescently staining myosin heavy chain in the engineered muscle tissue, wherein the engineered muscle tissue comprises myotubes.

Aspect 15: The method of aspect 14, further comprising measuring myotube length or myotube width in the engineered muscle tissue via the expression of the myosin heavy chain in the myotubes, wherein an increase in myotube length or myotube width compared to the myotube length or myotube width prior to contacting the engineered muscle tissue with the test substance indicates that the test substance increases myogenesis.

Aspect 16: The method of any one of aspects 3, 4 or 15, further comprising counting the number of nuclei per myotube in the engineered muscle tissue before contacting the engineered muscle tissue with the test substance and after contacting the engineered muscle tissue with the test substance; and comparing the number of nuclei per myotube in the engineered muscle tissue, wherein an increase in the number of nuclei per myotube in the engineered muscle tissue after contacting the engineered muscle tissue with the test substance indicates that the test substance increases myogenesis.

Aspect 17: The method of aspect 16, further comprising calculating a fusion index.

Aspect 18: The method of aspect 17, wherein when the fusion index is increased after contacting the engineered muscle tissue indicates that the test substance increases myogenesis.

Aspect 19: The method of any one of the preceding aspects, performing RNA sequencing analysis after contacting the engineered muscle tissue with the test substance.

Aspect 20: The method of any one of aspects 1 or 2, further comprising comparing the expression of the at least one mitochondrion gene or the at least one biological process gene in the engineered muscle tissue with the expression of the same at least one mitochondrion gene or the at least one biological process gene in the engineered muscle tissue after contacting the engineered muscle tissue with a reference compound.

Aspect 21: The method of aspect 20, wherein the reference compound is insulin-like growth factor-1 or 15-hydroxyprostaglandin dehydrogenase (SW033291).

Aspect 22: The method of any one of the preceding aspects, wherein the test substance is selected from the group consisting of an agent for treating or preventing disuse atrophy, an agent for treating or preventing sarcopenia, an agent for treating or preventing neurogenic atrophy, or an agent for treating or preventing disease-related atrophy.

Aspect 23: The method of any one of the preceding aspects, wherein the test substance is selected from the group consisting of an agent for treating or preventing disease associated with a decrease of slow-twitch muscle fibers, an agent for treating or preventing disease associated with an increase of in slow-twitch muscle fibers, an agent for treating or preventing disease associated with a decrease of fast-twitch muscle fibers, or an agent for treating or preventing disease associated with an increase of fast-twitch muscle fibers.

Aspect 24: The method of aspect 22, wherein the disease-related atrophy is cancer, chronic obstructive pulmonary disorder, diabetes, chronic kidney disease, heart failure, or a neurodegenerative disorder.

Aspect 25: The method of any one of the preceding aspects, wherein the test substance is an FDA-approved repurposed drug.

Aspect 26: The method of any one of the preceding aspects, wherein the test substance is not insulin-like growth factor-1.

Aspect 27: The method of any one of the preceding aspects, wherein the test substance is not 15-hydroxyprostaglandin dehydrogenase (15-PGDH-I, SW033291).

Aspect 28: The method of any one of the preceding aspects, further comprising performing a muscle contractility assessment of the engineered muscle tissue.

Aspect 29: The method of any one of the preceding aspects, further comprising comparing the muscle contractility of the engineered muscle tissue before contacting the engineered muscle tissue with the test substance and after contacting the engineered muscle tissue with the test substance, wherein an increase in muscle contractility of the engineered muscle tissue after contacting the engineered muscle tissue with the test substance indicates that the test substance increases myogenesis.

Aspect 30: A method of treating unloading/disuse atrophy in a subject in need thereof, the method comprising administering to the subject a therapeutically effective amount of 15-hydroxyprostaglandin dehydrogenase (15-PGDH-I, SW033291), thereby treating unloading/disuse atrophy in the subject.

Aspect 31: A method of reducing or ameliorating one or more symptoms of unloading/disuse atrophy in a subject in need thereof, the method comprising administering to the subject a therapeutically effective amount of 15-hydroxyprostaglandin dehydrogenase (15-PGDH-I, SW033291), thereby reducing or ameliorating one or more symptoms of unloading/disuse atrophy in the subject.

Aspect 32: The method of aspect 31, wherein the one or more symptoms of unloading/disuse atrophy is decreased muscle mass, limb size discrepancies, numbness, weakness, tingling, decreased muscle strength and/or size, difficulty with movement, balance, coordination or a combination thereof, pain, a decrease in slow-twitch fibers, an increase in fast-twitch fibers, insulin resistance, or a combination thereof.

Aspect 33: The method of any one of aspects 30-32, wherein the 15-PGDH-I is administered orally, intravenously, intra-articularly, or subcutaneously.

Aspect 34: The method of any one of aspects 30-33, wherein the subject is identified in need of treatment before the administering step.

Aspect 35: The method of any one of aspects 30-34, wherein the subject is a human.

Aspect 36: The method of any one of aspects 30-35, wherein the subject in need thereof has unloading/disuse atrophy.

Aspect 37: The method of any one of aspects 30-31, further comprising identifying the subject in need of treatment before the administering step as having an increase in expression of at least one mitochondrion gene or a decrease in at least one biological process gene compared to a control subject without unloading/disuse atrophy.

Aspect 38: The method of aspect 37, wherein the at least one mitochondrion gene is selected from the group of mitochondrially encoded cytochrome c oxidase III (MT-C03), fumarylacetoacetate hydrolase domain-containing protein 1 (FAHD1), lon protease homolog (LONP1), phosphoenolpyruvate carboxykinase 2 (PCK2), glutaredoxin-1 (GLRX), RAB32, Bcl-2 related ovarian killer (BOK), cytochrome C oxidase assembly factor 4 homolog (COA4), dehydrogenase/reductase 4 (DHRS4), fatty acid desaturase 1 (FADS1), EF-hand domain family member D1 (EFHD1), NX4, MTHFD2, and serum/glucocorticoid regulated kinase 1 (SGK1).

Aspect 39: The method of aspect 37, wherein the at least one biological process gene is selected from the group consisting of protocadherin gamma C3 (PDCHGC3), prostaglandin-endoperoxide synthase 1 (PTGS1), transforming growth factor beta induced (TGFB1), NYN domain and retroviral integrase containing (NYNRIN), neuroligin 2 (NLGN2), hemicentin-1 (HMCN1), dystonin (DST), zinc finger and BTB domain-containing 20 (ZBTB20), laminin alpha 2 (LAMA2), collagen type XII alpha 1 chain (COL12A1), proline rich coiled-coil 2C (PRRC2C), nuclear factor of activated T-cells 5 (NFAT5), golgin subfamily B member 1 (GOLGB1), SCUBE3, zinc finger protein 469 (ZNF469), heparan sulfate proteoglycan 2 (HSPG2), PH and SEC7 domain-containing protein 3 (PSD3), cluster of differentiation 109 (CD109), neuroblast differentiation-associated protein AHNAK (AHNAK), and neurogenic locus notch homolog protein 2 (NOTCH2).

Aspect 40: The method of any one of aspects 30-31, further comprising identifying the subject in need of treatment before the administering step as having a decrease in myotube length or myotube width compared to a control subject without unloading/disuse atrophy.

Aspect 41: The method of any one of aspects 1-29, further comprising exposing the engineered muscle tissue to simulated microgravity conditions.

Aspect 42: The method of any one of aspects 1-29, wherein the engineered muscle tissue is contacted with the test substance during exposure of the engineered muscle tissue to microgravity conditions.

Aspect 43: The method of aspect 42, wherein the microgravity conditions are simulated microgravity conditions.

Aspect 44: A system for performing the method of any one of aspects 1-29 or 41-43.

Aspect 45: The system of aspect 44, wherein the system comprises:
a clinostat;
an adapter that is receivable into the clinostat, wherein the adapter comprises a housing that defines at least one receptacle; and
an assembly comprising:
   a substrate; and
   a plurality of fibrils deposited on the substrate, wherein the plurality of fibrils comprise collagen, wherein the plurality of fibrils extend along an axis,
      wherein the assembly is receivable into a first receptacle of the at least one receptacle.

Aspect 46: The system of aspect 45, wherein the at least one receptacle comprises a plurality of receptacles, wherein the housing further defines a plurality of fluid conduits, wherein a respective fluid conduit of the plurality of fluid conduits is in communication with a corresponding receptacle of the plurality of receptacles.

Aspect 47: The system of aspect 46, wherein each fluid conduit is configured to receive fluid therethrough to permit the respective receptacle to be filled with media.

Aspect 48: The system of aspect 46, wherein the plurality of fluid conduits is a plurality of inlet conduits, wherein the housing further defines a plurality of outlet conduits, wherein a respective outlet conduit is in communication with a corresponding receptacle of the plurality of receptacles.

Aspect 49: A method comprising:
positioning an adapter within a clinostat, the adapter comprising a housing defining at least one receptacle;
positioning each assembly of at least one assembly into a respective receptacle of the at least one receptacle, wherein positioning each assembly of at least one assembly into the respective receptacle of the at least one receptacle comprises positioning a first assembly in a first receptacle of the at least one receptacle of the adapter, wherein each assembly of the at least one assembly comprises:
   a substrate; and
   a plurality of fibrils deposited on the substrate, wherein the plurality of fibrils comprise collagen, wherein the plurality of fibrils extend along an axis;
operating the clinostat to simulate microgravity; and
flowing a first media into the first receptacle.

Aspect 50: The method of aspect 49, further comprising:
positioning a second assembly of the at least one assembly in a second receptacle of the at least one receptacle of the adapter; and
flowing a second media into the second receptacle, wherein the second media is different from the first media.

Aspect 51: The method of aspect 50, further comprising:
positioning a third assembly of the at least one assembly in a third receptacle of the at least one receptacle of the adapter;
flowing a third media into the second receptacle, wherein the third media is different from the first media and the second media;
positioning a fourth assembly of the at least one assembly in a fourth receptacle of the at least one receptacle of the adapter; and
flowing a fourth media into the second receptacle, wherein the fourth media is different from the first, second, and third media.

Aspect 52: The method of any one of aspects 49-51, wherein the first assembly has a plurality of myoblasts seeded on the plurality of fibrils.

Aspect 53: The method of aspect 52, wherein the plurality of myoblasts seeded on the plurality of fibrils cooperate to form a plurality of different samples.

What is claimed is:

1. A system comprising:
a clinostat;
an adapter that is receivable into the clinostat, wherein the adapter comprises a housing that defines at least one receptacle; and
an assembly comprising:
a substrate; and
a plurality of fibrils deposited on the substrate, wherein the plurality of fibrils comprise collagen, wherein the plurality of fibrils extend along an axis,
wherein the assembly is receivable into a first receptacle of the at least one receptacle.

2. The system of claim 1, wherein the at least one receptacle comprises a plurality of receptacles, wherein the housing further defines a plurality of fluid conduits, wherein a respective fluid conduit of the plurality of fluid conduits is in communication with a corresponding receptacle of the plurality of receptacles.

3. The system of claim 2, wherein each fluid conduit is configured to receive fluid therethrough to permit the respective receptacle to be filled with media.

4. The system of claim 2, wherein the plurality of fluid conduits is a plurality of inlet conduits, wherein the housing further defines a plurality of outlet conduits, wherein a respective outlet conduit is in communication with a corresponding receptacle of the plurality of receptacles.

5. A method comprising:
positioning an adapter within a clinostat, the adapter comprising a housing defining at least one receptacle;
positioning each assembly of at least one assembly into a respective receptacle of the at least one receptacle, wherein positioning each assembly of at least one assembly into the respective receptacle of the at least one receptacle comprises positioning a first assembly in a first receptacle of the at least one receptacle of the adapter, wherein each assembly of the at least one assembly comprises:
a substrate; and
a plurality of fibrils deposited on the substrate, wherein the plurality of fibrils comprise collagen, wherein the plurality of fibrils extend along an axis;
operating the clinostat to simulate microgravity; and
flowing a first media into the first receptacle.

6. The method of claim 5, further comprising:
positioning a second assembly of the at least one assembly in a second receptacle of the at least one receptacle of the adapter; and
flowing a second media into the second receptacle, wherein the second media is different from the first media.

7. The method of claim 6, further comprising:
positioning a third assembly of the at least one assembly in a third receptacle of the at least one receptacle of the adapter;
flowing a third media into the second receptacle, wherein the third media is different from the first media and the second media;
positioning a fourth assembly of the at least one assembly in a fourth receptacle of the at least one receptacle of the adapter; and
flowing a fourth media into the second receptacle, wherein the fourth media is different from the first, second, and third media.

8. The method of claim 5, wherein the first assembly has a plurality of myoblasts seeded on the plurality of fibrils.

9. The method of claim 8, wherein the plurality of myoblasts seeded on the plurality of fibrils cooperate to form a plurality of different samples.

10. A method for screening a test substance affecting myogenesis, the method comprising:
a) providing an engineered muscle tissue, wherein the engineered muscle tissue has been exposed to microgravity conditions;
b) contacting the engineered muscle tissue with the test substance;
c) determining the expression of at least one mitochondrion gene or at least one biological process gene in the engineered muscle tissue after step b); and
d) comparing the expression of the at least one mitochondrion gene or the at least one biological process gene in the engineered muscle tissue in step b) to the expression of the at least one mitochondrion gene or the at least one biological process gene in the engineered muscle tissue prior to step b),
wherein a decrease in expression of the at least one mitochondrion gene indicates that the test substance increases myogenesis or wherein an increase in the expression of the at least one biological process gene indicates that the test substance increases myogenesis.

11. The method of claim 10, wherein the engineered muscle tissue is engineered skeletal muscle tissue.

12. The method of claim 10, wherein the engineered muscle tissue comprises a plurality of myoblasts.

13. The method of claim 10, wherein the microgravity conditions comprise exposing the engineered muscle tissue to 10-3 g for 7 to 14 days.

14. The method of claim 10, wherein the test substance is applied to the engineered muscle tissue for 7 to 14 days.

15. The method of claim 10, wherein the at least one mitochondrion gene is selected from the group of mitochondrially encoded cytochrome c oxidase III (MT-CO3), fumarylacetoacetate hydrolase domain-containing protein 1 (FAHD1), lon protease homolog (LONP1), phosphoenolpyruvate carboxykinase 2 (PCK2), glutaredoxin-1 (GLRX), RAB32, Bcl-2 related ovarian killer (BOK), cytochrome C oxidase assembly factor 4 homolog (COA4), dehydrogenase/reductase 4 (DHRS4), fatty acid desaturase 1 (FADS1), EF-hand domain family member D1 (EFHD1), NX4, MTHFD2, and serum/glucocorticoid regulated kinase 1 (SGK1).

16. The method of claim 10, wherein the at least one biological process gene is selected from the group consisting of protocadherin gamma C3 (PDCHGC3), prostaglandin-endoperoxide synthase 1 (PTGS1), transforming growth factor beta induced (TGFB1), NYN domain and retroviral integrase containing (NYNRIN), neuroligin 2 (NLGN2), hemicentin-1 (HMCN1), dystonin (DST), zinc finger and BTB domain-containing 20 (ZBTB20), laminin alpha 2 (LAMA2), collagen type XII alpha 1 chain (COL12A1), proline rich coiled-coil 2C (PRRC2C), nuclear factor of activated T-cells 5 (NFAT5), golgin subfamily B member 1 (GOLGB1), SCUBE3, zinc finger protein 469 (ZNF469), heparan sulfate proteoglycan 2 (HSPG2), PH and SEC7 domain-containing protein 3 (PSD3), cluster of differentiation 109 (CD109), neuroblast differentiation-associated protein AHNAK (AHNAK), and neurogenic locus notch homolog protein 2 (NOTCH2).

17. The method of claim 10, further comprising performing a proteomic analysis of the engineered muscle tissue.

18. The method of claim 17, wherein the proteomic analysis comprises determining the amount of at least one protein selected from the group consisting of Eotaxin-3 (CCL26), C-X-C motif chemokine ligand 16 (CXCL16), growth differentiation factor 15 (GDF-15), tumor necrosis factor superfamily member 14 (LIGHT/TNSF14), and pupoid fetus (PF), wherein when the amount of Eotaxin-3 (CCL26), C-X-C motif chemokine ligand 16 (CXCL16), growth differentiation factor 15 (GDF-15), tumor necrosis factor superfamily member 14 (LIGHT/TNSF14), or pupoid fetus (PF) is decreased compared to the amount of the same protein present prior to contacting the engineered muscle tissue with the test substance indicates that the test substance increases myogenesis.

19. The method of claim 17, wherein the proteomic analysis comprises determining the amount of at least one protein selected from the group consisting of bone morphogenetic protein 4 (BMP-4), Resistin, C-X-C motif chemokine ligand 12 (CXCL12/SDF-1b), interleukin-16 (IL-16), and CD40, wherein when the amount of bone morphogenetic protein 4 (BMP-4), Resistin, C-X-C motif chemokine ligand 12 (CXCL12/SDF-1b), interleukin-16 (IL-16), or CD40 is increased compared to the amount of the same protein present prior to contacting the engineered muscle tissue with the test substance indicates that the test substance increases myogenesis.

20. The method of claim 10, further comprising exposing the engineered muscle tissue to simulated microgravity conditions.

21. The method of claim 20, wherein the engineered muscle tissue contacts the test substance during exposure of the engineered muscle tissue to simulated microgravity conditions.
